(19) United States
(12) Patent Application Publication
(10) Pub. No.: US 2013/0005730 A1
(43) Pub. Date: Jan. 3, 2013

(54) NOVEL 1,3-OXAZOLIDINE COMPOUNDS AND THEIR USE AS RENIN INHIBITORS

(76) Inventors: Piaoyang Sun, Jiangsu (CN); Mikhajlovich Antonov Dmitry, Skogas (SE); Xiao Xiong Zhou, Huddinge (SE)

(21) Appl. No.: 13/508,830

(22) PCT Filed: Oct. 29, 2010
(86) PCT No.: PCT/SE2010/051181

§ 371 Date: Aug. 14, 2012

Related U.S. Application Data
(60) Provisional application No. 61/259,362, filed on Nov. 9, 2009.

(30) Foreign Application Priority Data
Nov. 9, 2009 (SE) 0950845-8

(51) Int. Cl. A61K 31/421; C07D 413/12; A61K 31/5377; A61K 31/4439; A61K 31/454; A61P 13/12; A61K 31/439; A61P 9/12; A61P 9/04; A61P 27/06; A61P 9/10; C07D 263/06; C07D 453/02

(52) U.S. Cl. 514/236.8; 548/215; 514/374; 544/137; 546/271.4; 514/340; 546/209; 514/326; 546/133; 514/305; 548/236

(57) ABSTRACT

The present invention relates to certain novel 1,3-oxazolidine compounds of formula (I), to processes for making such compounds and to their utility as renin inhibitors or prodrugs of renin inhibitors.

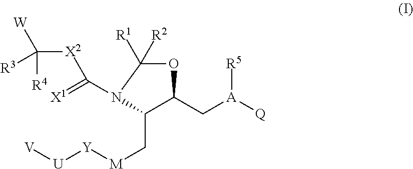

### FIELD OF THE INVENTION

The present invention relates to certain novel 1,3-oxazolidine compounds, to processes for making such compounds and to their utility as renin inhibitors, precursors of renin inhibitors or prodrugs of renin inhibitors.

### BACKGROUND OF THE INVENTION

Hypertension is one of the major cardiovascular diseases, which are responsible for the millions death worldwide each year. The renin-angiotensin system (RAS) plays a key role in the maintenance of hemodynamic integrity via modulating regulator of blood pressure and body fluid volume in response to a broad range of physiological and environmental variations.

Renin is a proteolytic enzyme that metabolizes angiotensinogen to angiotensin I. Angiotensin I can be subsequently cleaved by Angiotensin-converting enzyme (ACE), producing angiotensin II, which is the effector of the RAS system and mediates its physiological function via the interaction with its receptors. The blockade of RAS is an effective therapeutic approach in the treatment of hypertension and the intervention of other pathogenesis of cardiovascular and renal disorder.

Direct renin inhibition has long been suggested as one of the means for the inhibition of the RAS. Renin (EC 3.4.99.19) was first discovered in 19^{th} century and its functions in the RAS was established thereafter. Renin controls the first step of the renin-angiotension system and catalyzes the cleavage of angiotensinogen at a unique site, releasing the decapeptide angiotensin. Renin is a highly specific protease and its only known natural substrate is angiotensinogen. Due to the high specificity and its rate-limiting nature in the RAS cascade, renin is regarded as one of the most attractive targets for the inhibition of the RAS, and enormous efforts have been made to develop potent and safe renin inhibitors.

The compound (2S,4S,5S,7S)—N-(2-Carbamoyl-2-methylpropyl)-5-amino-4-hydroxy-2,7-diisopropyl-8-[4-methoxy-3-(3-methoxypropoxy)phenyl]-octanamide, which is disclosed in EP-A-678503, more commonly known under the name aliskiren, is one of the most important renin inhibitors, and the first renin inhibitor that has been approved for clinical use in the treatment of hypertension and related diseases. The chemical structure of aliskiren is shown in Figure 1.

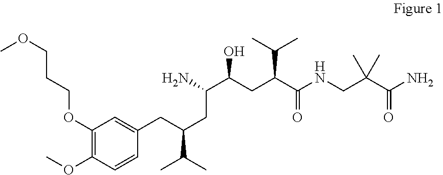 [SMILES: COCCCOc1cc(C[C@@H](C[C@H](N)[C@@H](O)C[C@H](C(=O)NCC(C)(C)C(N)=O)C(C)C)C(C)C)ccc1OC]

Aliskiren is being used in monotherapy for hypertension, and studies for the combination therapies such as with diuretics, ACE inhibitors and angiotensin receptor blockers are under way. Aliskiren is a potent inhibitor of renin with a Ki in the sub-nanomolar level. Aliskiren has a very good safety profile.

However, the renin inhibitors are known to have unfavorable properties such as an unfavorable pharmacokinetic profile. For instance, they exhibit low oral bioavailability, interaction with efflux system and so on.

In the journal Clinical Pharmacokinetics, 2008, 47, 515-531, it is disclosed that aliskiren has a low oral bioavailability of about 2.6%. Many other renin inhibitors were also reported to have bad pharmacokinetic properties.

It is an object of the present invention to overcome or at least mitigate some of the disadvantages associated with renin inhibitors mentioned above.

### DESCRIPTION OF THE INVENTION

Thus, the present invention relates to a compound of formula (I)

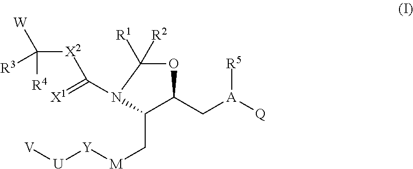

wherein
R¹ and R² independently represent
H, C₁-C₆alkyl, C₃-C₆cycloalkyl or C₃-C₆cycloalkyl-C₁-C₃alkyl, wherein said C₁-C₆alkyl, C₃-C₆cycloalkyl or C₃-C₆cycloalkyl-C₁-C₃alkyl is optionally substituted by one or more substituents independently selected from halogen, CN, NH(C₁-C₆alkyl), N(C₁-C₆alkyl)₂, C₁-C₆alkyl and C₁-C₆alkoxy;
   or R¹ and R² together with the carbon to which they are bonded form
a C₃-C₆cycloalkyl or a 4-6 membered heterocyclyl, wherein said C₃-C₆cycloalkyl or 4-6 membered heterocyclyl is optionally substituted by one or more substituents independently selected from halogen, CN, NH(C₁-C₆alkyl), N(C₁-C₆alkyl)₂, C₁-C₆alkyl and C₁-C₆alkoxy;
   R³ and R⁴ independently represent
H, C₁-C₈alkyl, C₂-C₈alkenyl, C₂-C₈alkynyl, C₃-C₆cycloalkyl, C₃-C₆cycloalkyl-C₁-C₆alkyl, C₁-C₈alkoxy, C₁-C₈alkoxy-C₁-C₆alkyl, aryl-C₁-C₆alkyl, heterocyclyl-C₁-C₆alkyl, aryl, aryloxy, heterocyclyl or heterocyclyloxy, wherein said C₁-C₈alkyl, C₂-C₈alkenyl, C₂-C₈alkynyl, C₃-C₆cycloalkyl, C₃-C₆cycloalkyl-C₁-C₆alkyl, C₁-C₆alkoxy, C₁-C₈alkoxy-C₁-C₆alkyl, aryl-C₁-C₆alkyl, heterocyclyl-C₁-C₆alkyl, aryl, aryloxy, heterocyclyl or heterocyclyloxy is optionally substituted by one or more substituents independently selected from halogen, OH, CN, NO₂, NH₂, NH(C₁-C₆alkyl), N(C₁-C₆alkyl)₂, C₁-C₆alkyl, C₁-C₆alkoxy and C₃-C₆cycloalkyl;
   or R³ and R⁴ together with the carbon to which they are bonded form
a C₃-C₈cycloalkyl or a 4-8 membered heterocyclyl, wherein said C₃-C₃cycloalkyl or 4-8 membered heterocyclyl is optionally substituted by one or more substituents independently selected from halogen, OH, CN, NO₂, NH₂, NH(C₁-C₃alkyl), N(C₁-C₃alkyl)₂, C₁-C₃alkyl, C₃-C₆cycloalkyl and C₁-C₃alkoxy;
   X¹ represents
O or S;
   X² represents
O or S;
   W represents
H, R⁶X¹—, C₂-C₆alkyl, halogen, (OH)₂P(O)O, [R^{a}C(O)OCH₂O]₂P(O)O, or [R^{a}C(O)OCH(C₁-C₃alkyl)O]₂P(O)O, [R^{a}C(O)SCH₂CH₂O]₂P(O)O;
   R^{a} represents
C₁-C₆alkyl, C₃-C₆cycloalkyl, C₂-C₆-alkenyl, heterocyclyl or aryl, wherein said C₁-C₆alkyl, C₃-C₆cycloalkyl, C₂-C₆-alkenyl, heterocyclyl or aryl is optionally substituted by one or more substituents independently selected from halogen, OH, NH₂, NH(C₁-C₃alkyl), N(C₁-C₃alkyl)₂, C₁-C₃alkyl, C₁-C₃alkoxy, aryl and heterocyclyl;

### R⁶ is —C(═X¹)TZ;

T represents
O, S, NH, N(C₁-C₃alkyl) or a single bond;
   Z represents
C₁-C₁₈alkyl, C₂-C₁₈alkenyl, C₂-C₁₈alkynyl, C₃-C₈cycloalkenyl, C₄-C₈cycloalkynyl, aryl, heterocyclyl, C₃-C₈cycloalkyl, C₁-C₁₈alkyl-heterocyclyl, tetrazolyl-biphenyl-methyl-heterocyclyl, tetrazolyl-biphenyl-methyl-heterocyclylmethyl, tetrazolyl-biphenyl-methyl-amino-C₁-C₆alkyl, oxadiazolyl-biphenyl-methyl-heterocyclyl, heterocyclylmethyl-aryl, C₁-C₆alkyl-aryl or C₁-C₆alkyl-C₃-C₈cycloalkyl, wherein said C₁-C₁₈alkyl, C₂-C₁₈alkenyl, C₂-C₁₈alkynyl, C₃-C₈cycloalkenyl, C₄-C₈cycloalkynyl, aryl, heterocyclyl, C₃-C₈cycloalkyl, C₁-C₁₈alkyl-heterocyclyl, tetrazolyl-biphenyl-methyl-heterocyclyl, tetrazolyl-biphenyl-methyl-heterocyclylmethyl, tetrazolyl-biphenyl-methyl-amino-C₁-C₆alkyl, oxadiazolyl-bephenyl-methyl-heterocyclyl, heterocyclylmethyl-aryl, C₁-C₆alkyl-aryl or C₁-C₆alkyl-C₃-C₈cycloalkyl is optionally substituted by one or more substituents independently selected from halogen, OH, CN, oxo, N₃, NO₂, NH₂, NH(C₁-C₆alkyl), N(C₁-C₆alkyl)₂, C₁-C₆alkanoylNH, C₂-C₆alkoxycarbonylNH, C₁-C₆alkanoyl, C₆alkanoyloxy, COOH, (OH)₂P(O)O, [R^{a}C(O)OCH₂O]₂P(O)O, [R^{a}C(O)OCH(C₁-C₃alkyl)O]₂P(O)O, [R^{a}C(O)SCH₂CH₂O]₂P(O)O, NH₂C(O), C₁-C₆alkyl, C₂-C₆alkenyl, C₂-C₆alkynyl, C₁-C₆alkoxy, C₁-C₆alkoxycarbonyl, C₁-C₆alkoxycarbonylNH, NH₂C₁-C₆alkyl, C₁-C₆alkoxycarbonylNHC₁-C₃alkyl, aryl-C₁-C₄alkylcarbonylNH, C₃-C₆cycloalkyl, C₃-C₆cycloalkenyl, C₃-C₆cycloalkoxy, C₃-C₆cycloalkenyloxy, C₁-C₃alkoxy-C₁-C₆alkoxy, aryl, aryloxy, heterocyclyloxy and heterocyclyl;
   M represents
O, S, SO₂, N(R⁷) or

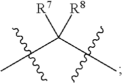

R⁷ represents
H, C₁-C₆alkyl, C₂-C₆alkenyl, C₂-C₆alkynyl, C₃-C₆cycloalkyl, aryl, heterocyclyl or aryl(C₁-C₆)alkyl, wherein said C₁-C₆alkyl, C₂-C₆alkenyl, C₂-C₆alkynyl, C₃-C₆cycloalkyl, aryl, heterocyclyl or aryl(C₁-C₆)alkyl is optionally substituted by one or more substituents independently selected from halogen, C₃-C₆cycloalkyl or C₁-C₆alkyl, wherein said C₃-C₆cycloalkyl or C₁-C₆alkyl is optionally substituted by one or more substituents selected from halogen, aryl and heterocyclyl;
   R⁸ represents
H, OH, halogen, C₁-C₆alkyl or C₁-C₆alkoxy; or R⁷ and R⁸ together with the carbon atom to which they are bonded form a C₃-C₈cycloalkyl;
   Y represents
a single bond, CH₂, C₂-C₆alkanoyloxymethylene, O, S, SO, SO₂, NH, N(C₁-C₄alkyl), C(O), or CH(OH);
   U represents
a single bond, CH₂, C(O), C(O)NH, NHC(O), NH or N(C₁-C₄alkyl);
   V represents
a 3-18-membered saturated, partially unsaturated or aromatic mono-, bi- or tricyclic system, said system is a carbocyclic ring system or a heterocyclic ring system selected from C₃-C₁₂cycloalkyl, C₃-C₁₂cycloalkenyl, C₄-C₁₂cycloalkynyl, heterocyclyl and aryl, wherein said system is optionally substituted with one, two, three or four substituents independently selected from halogen, OH, CN, oxo, COOH, CF₃, NO₂, NH₂, NH(C₁-C₆alkyl), N(C₁-C₆alkyl)₂, C₁-C₆alkoxy, C₁-C₆alkoxy-C₁-C₆alkoxy, NH₂C(O), C₃-C₆cycloalkyl, C₂-C₆alkenyl, C₃-C₆cycloalkoxy-C₁-C₆alkoxy, C₃-C₈cycloalkyl-C₁-C₆alkoxy, dioxalanyl, hydroxyl-C₂-C₇alkoxy, haloC₂-C₇alkoxy, carbamoyloxy-C₂-C₇alkoxy, [(C₅H₅N)NHC(O)]C₁-C₇alkoxy, C₃-C₆cycloalkoxy, C₂-C₇alkenyloxy, C₁-C₆alkanoyloxy, C₁-C₆alkoxycarbonyl, C₁-C₃alkoxycarbonyl, C₁-C₆alkylenedioxy, aryl, phenoxy, phenylthio, pyridyl and C₁-C₆alkyl, wherein said C₁-C₆alkyl is optionally substituted by C₃-C₆cycloalkoxy, C₁-C₆alkoxy, (C₅H₅N)C(O)NH, NH₂C(O), NH(C₁-C₃alkyl)C(O), N(C₁-C₃)₂C(O), NH₂C(O)C₁-C₃alkoxy, NH(C₁-C₃alkyl)C(O)C₁-C₃alkoxy, N(C₁-C₃alkyl)₂C(O)C₁-C₃alkoxy or phenyl;
   A represents
CH or N;
   R⁵ represents
H, C₁-C₆alkyl, C₂-C₆alkenyl, C₂-C₆alkynyl, C₃-C₆cycloalkyl or C₁-C₆alkoxy, wherein said C₁-C₆alkyl, C₂-C₆alkenyl, C₂-C₆alkynyl, C₃-C₆cycloalkyl or C₁-C₆alkoxy is optionally substituted by one or more of substituents independently selected from halogen, OH, C₃-C₆cycloalkyl, C₁-C₆alkoxy and aryl;
   Q represents
C₁-C₈alkyl, C₃-C₈cycloalkyl, NH(C₁-C₈alkyl)C(O)C₁-C₆alkyl, N(C₁-C₈alkyl)₂C(O)C₁-C₆alkyl, aryl, heterocyclyl or heterocyclyl-C₁-C₄alkyl;
   or Q is selected from the group of partial structures consisting of E1 and E2

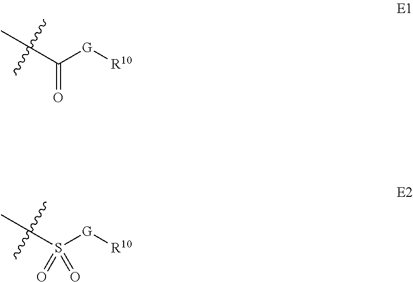

G represents

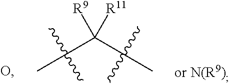

R¹¹ represents
H or C₁-C₆alkyl;
   or R⁵, Q and A, wherein A is N, form a 3-18-membered saturated, partially unsaturated or aromatic mono-, bi- or tricyclic ring system, wherein said system is optionally substituted by one, two, three or four substituents independently selected from halogen, OH, oxo, CN, C₁-C₆alkyl, C₃-C₈cycloalkyl, C₃-C₈cycloalkanoyl, C₁-C₈alkanoyl, aryl-C₁-C₆alkanoyl, C₁-C₈alkoxycarbonyl, C₁-C₈alkyl-SO₂, heterocyclylSO₂, aryl and heterocyclyl;
   R⁹ represents
H, C₁-C₆alkyl, C₃-C₈cycloalkyl, C₂-C₆alkenyl or C₁-C₆alkoxy, wherein said C₁-C₆alkyl, C₃-C₈cycloalkyl, C₂-C₆alkenyl or C₁-C₆alkoxy is optionally substituted by one or more halogen;
   R¹⁰ represents
H, C₁-C₁₂alkyl, C₂-C₁₂alkenyl, C₃-C₁₂cycloalkyl, C₃-C₁₂cycloalkenyl, heterocyclyl or aryl, wherein said C₁-C₁₂alkyl, C₂-C₁₂alkenyl, C₃-C₁₂cycloalkyl, C₃-C₁₂cycloalkenyl, heterocyclyl or aryl is optionally substituted by one or more substituents independently selected from halogen, OH, CN, NO₂, C₁-C₈alkoxy, C₃-C₆cycloalkyl, aryloxy, heterocycloxy, NH₂C(O), NH(aryl), NH(heterocyclyl), NH(aryl)C(O), NH(heterocyclyl)C(O), C₁-C₈alkyl-C(O)NH, arylC(O)NH, C₁-C₈alkanoyl, C₁-C₆alkoxyC(O), C₁-C₈alkylSO₂, aryl-SO₂, aryl and heterocyclyl;

### or R¹⁰ is

C₁-C₈alkyl or C₁-C₈alkenyl, wherein said C₁-C₈alkyl or C₁-C₈alkenyl is optionally substituted by NH₂C(O), NH(C₁-C₈alkyl)C(O), NH(C₃-C₈cycloalkyl)C(O), NH(C₃-C₆-alkenyl)C(O), N(C₁-C₆alkyl)₂C(O), C₁-C₆alkoxycarbonylNHC(O), N(C₃-C₈cycloalkyl)₂C(O), N(C₃-C₆cycloalkyl)(C₁-C₃alkyl)C(O), N(heterocyclyl)(C₁-C₆alkyl)C(O), NH₂C(S) or NH(C₁-C₈alkyl)C(S);
or R¹⁰ is
   C₁-C₆alkyl or C₂-C₆alkenyl, wherein said C₁-C₆alkyl or C₂-C₆alkenyl is optionally substituted with NH₂C(O)C₃-C₆cycloalkyl;
   or R⁹ and R¹⁰ together with the atom of G to which R⁹ and R¹⁰ are bonded form
a 3-18-membered saturated, partially unsaturated or aromatic mono-, bi- or tricyclic system, said system is a carbocyclic ring system or a heterocyclic ring system, wherein said system is optionally substituted by one, two, three or four substituents independently selected from halogen, OH, oxo, C₁-C₆alkyl, C₃-C₈cycloalkyl, C₁-C₆alkoxy, C₃-C₈cycloalkoxy, C₁-C₈alkanoyl, C₁-C₈alkanoyloxy, aryl-C₁-C₆-alkanoyl, C₁-C₈-alkoxycarbonyl, C₁-C₈alkyl-SO₂—, heterocyclyl-SO₂, aryl and heterocyclyl;
   with the proviso that R⁴ is not aryl when R³ and W are H;
   and with the proviso that R³ is not aryl when R⁴ and W are H;
   or a pharmaceutically acceptable salt thereof.

In one embodiment of the present invention, there is provided a compound of formula (I) wherein

Z represents
C₁-C₁₈alkyl, C₂-C₁₈alkenyl, C₂-C₁₈alkynyl, C₃-C₈cycloalkenyl, C₄-C₈cycloalkynyl, aryl, heterocyclyl or C₃-C₈cycloalkyl, wherein said C₁-C₁₈alkyl, C₂-C₁₈alkenyl, C₂-C₁₈alkynyl, C₃-C₈cycloalkenyl, C₄-C₈cycloalkynyl, aryl, heterocyclyl or C₃-C₈cycloalkyl is optionally substituted by one or more of the substituents independently selected from: halogen, OH, CN, oxo, N₃, NO₂, NH₂, NH(C₁-C₆alkyl), N(C₁-C₆alkyl)₂, C₁-C₆alkanoylNH, C₂-C₆alkoxycarbonylNH, C₁-C₆-alkanoyl, C₁-C₆alkanoyloxy, COOH, (OH)₂P(O)O, [R^{a}C(O)OCH₂O]₂P(O)O, [R^{a}C(O)OCH(C₁-C₃alkyl)O]₂P(O)O, [R^{a}C(O)SCH₂CH₂O]₂P(O)O, NH₂C(O), C₁-C₆alkyl, C₂-C₆alkenyl, C₂-C₆alkynyl, C₁-C₆alkoxy, C₁-C₆alkoxycarbonyl, C₃-C₆cycloalkyl, C₃-C₆cycloalkenyl, C₃-C₆cycloalkoxy, C₃-C₆cycloalkenyloxy, C₁-C₃alkoxy-C₁-C₆alkoxy, aryl, aryloxy, heterocyclyloxy and heterocyclyl.

In one embodiment of the present invention, there is provided a compound of formula (I)

wherein

### X¹ is O;

### X² is O or S; and

### W is R⁶O—.

In another embodiment of the present invention, there is provided a compound of formula (I)

wherein

### X² is O.

In one embodiment of the present invention, there is provided a compound of formula (I)

wherein

### X¹ is O;

### X² is O;

### M is

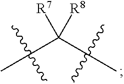

and
U is a single bond.

In one embodiment of the present invention, there is provided a compound of formula (I)

wherein

### X¹ is O;

### X² is O;

### W is R⁶O—;

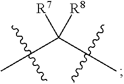

### M is

U is a single bond;

### A is CH and

### Q is E1.

In one embodiment of the present invention, there is provided a compound of formula (I)

wherein
R⁵ is C₁-C₆alkyl or C₃-C₆cycloalkyl.

In one embodiment of the present invention, there is provided a compound of formula (I)

wherein

### V—Y—U-M is:

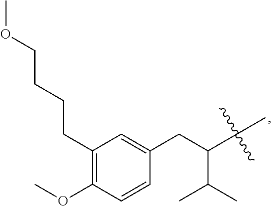 [SMILES: *C(Cc1ccc(OC)c(CCCCOC)c1)C(C)C]

R⁵ is isopropyl;
Q is E1, wherein G is N(R⁹); and

### R⁹ is H.

In one embodiment of the present invention, there is provided a compound of formula (I)

wherein

### V—U—Y-M is

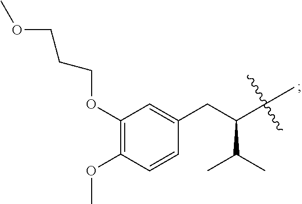 [SMILES: *[C@H](Cc1ccc(OC)c(OCCCOC)c1)C(C)C]

### A(R⁵)Q is

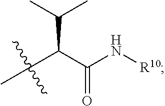

and

### R¹⁰ is

C₁-C₆alkyl, NH₂C(O)C₂-C₆alkyl, NH(C₁-C₆alkyl)C(O)C₂-C₅alkyl, N(C₁-C₆alkyl)₂C(O)C₂-C₅alkyl, C₁-C₆alkoxycarbonylNHC(O)—C₂-C₆alkyl, aryl-C₁-C₃alkyl, C₃-C₆cycloalkyl-C₁-C₂alkyl, NH₂C(O)cyclopropyl, C₃-C₆cycloalkyl or aryl.

In one embodiment of the present invention, there is provided a compound of formula (I)

wherein

### V—U—Y-M is

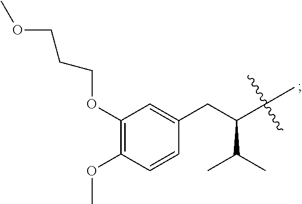 [SMILES: *[C@H](Cc1ccc(OC)c(OCCCOC)c1)C(C)C]

and

### A(R⁵)Q is

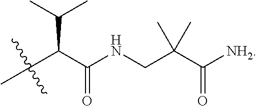 [SMILES: *[C@H](C(=O)NCC(C)(C)C(N)=O)C(C)C]

In one embodiment of the present invention, there is provided a compound of formula (I)

wherein

### X¹ is O;

### X² is O;

### W is R⁶O—;

### V—U—Y-M is

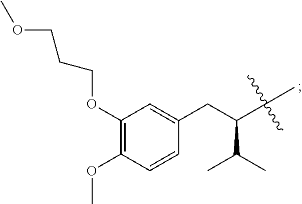 [SMILES: *[C@H](Cc1ccc(OC)c(OCCCOC)c1)C(C)C]

and

### A(R⁵)Q is

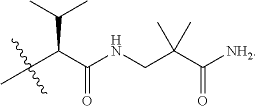 [SMILES: *[C@H](C(=O)NCC(C)(C)C(N)=O)C(C)C]

In one embodiment of the present invention, there is provided a compound of formula (I)

wherein

### V—U—Y-M is

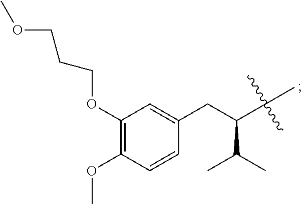 [SMILES: *[C@H](Cc1ccc(OC)c(OCCCOC)c1)C(C)C]

### A(R⁵)Q is

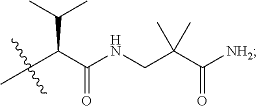 [SMILES: *[C@H](C(=O)NCC(C)(C)C(N)=O)C(C)C]

R¹ and R² independently represent
H, methyl or ethyl;
   or R¹ and R² together with the carbon to which they are bonded form
a C₃-C₆cycloalkyl or a 4-6 membered heterocyclyl, wherein said C₃-C₆cycloalkyl or 4-6 membered heterocyclyl is optionally substituted by one or more substituents independently selected from halogen, CN, NH(C₁-C₃-alkyl), N(C₁-C₃alkyl)₂, C₁-C₃alkyl and C₁-C₃alkoxy;
   R³ and R⁴ independently represent
H or methyl;
   or R³ and R⁴ together with the carbon to which they are bonded form a
C₃-C₈cycloalkyl or a 4-8 membered heterocyclyl, wherein said C₃-C₈cycloalkyl or 4-8 membered heterocyclyl is optionally substituted by one or more substituents independently selected from halogen, OH, NH₂, NH(C₁-C₃alkyl), N(C₁-C₃alkyl)₂, C₁-C₃alkyl or C₁-C₃alkoxy;
   X¹ iso;

### X² is O;

### W is R⁶O—;

### R⁶ is —C(═X¹)TZ

T is a single bond or O;
Z represents
C₁-C₁₈alkyl, C₂-C₁₈alkenyl, C₂-C₁₈alkynyl, C₃-C₈cycloalkenyl, C₄-C₈cycloalkynyl, aryl, heterocyclyl or C₃-C₈cycloalkyl, wherein said C₁-C₁₈alkyl, C₂-C₁₈alkenyl, C₂-C₁₈alkynyl, C₃-C₈cycloalkenyl, C₄-C₈cycloalkynyl, aryl, heterocyclyl or C₃-C₈cycloalkyl is optionally substituted by one or more of the substituents independently selected from: halogen, OH, CN, oxo, N₃, NO₂, NH₂, NH(C₁-C₆alkyl), N(C₁-C₆alkyl)₂, C₁-C₆alkanoylNH, C₂-C₆alkoxycarbonylNH, C₁-C₆alkanoyl, C₁-C₆alkanoyloxy, COOH, (OH)₂P(O)O, [R^{a}C(O)OCH₂O]₂P(O)O, [R^{a}C(O)OCH(C₁-C₃alkyl)O]₂P(O)O, [R^{a}C(O)SCH₂CH₂O]₂P(O)O, NH₂C(O)—, C₁-C₆alkyl, C₂-C₆alkenyl, C₂-C₆alkynyl, C₁-C₆alkoxy, C₁-C₆alkoxycarbonyl, C₃-C₆cycloalkyl, C₃-C₆cycloalkenyl, C₃-C₆cycloalkoxy, C₃-C₆cycloalkenyloxy, C₁-C₃alkoxy-C₁-C₆alkoxy-, aryl, aryloxy, heterocyclyloxy and heterocyclyl.

In one embodiment of the present invention, there is provided a compound of formula (I)

wherein
R¹ and R² independently represent
H or C₁-C₂alkyl;
R³ and R⁴ independently represent H or C₁-C₃alkyl;
   X¹ represents O;
   X² represents O;
   W represents R⁶X¹— or H;
   R⁶ represents —C(═X¹)TZ;
   T represents O or a single bond;
   Z represents
C₁-C₈alkyl, C₂-C₁₈alkenyl, C₃-C₈cycloalkyl, aryl, heterocyclyl, or C₁-C₆alkyl-C₃-C₈cycloalkyl, wherein said C₁-C₈alkyl, C₂-C₁₈alkenyl, C₃-C₈cycloalkyl, aryl, heterocyclyl, C₁-C₆alkyl-aryl or C₁-C₆alkyl-C₃-C₈cycloalkyl is optionally substituted by one or two substituents independently selected from halogen, OH, oxo, NH₂, N(C₁-C₆alkylC₂, C₂-C₄alkoxycarbonylNH, C₁-C₆alkyl, C₁-C₆alkoxy, C₁-C₆alkoxycarbonylNH, C₁-C₆alkoxycarbonyl, C₃-C₆cycloalkyl, C₁-C₃alkoxy-C₁-C₆alkoxy-, heterocyclyloxy, heterocyclyl, NH₂C₁-C₆alkyl, C₁-C₆alkoxycarbonylNHC₁-C₃alkyl and arylC₁-C₄alkylcarbonylNH;

### V—U—Y-M is

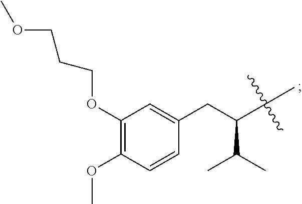 [SMILES: *[C@H](Cc1ccc(OC)c(OCCCOC)c1)C(C)C]

### A(R⁵)Q is

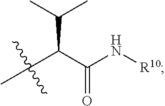

R¹⁰ represents C₁-C₄alkyl, said C₁-C₄alkyl is optionally substituted by one NH₂C(O).

In one embodiment of the present invention, there is provided a compound of formula (I)

wherein
R¹ and R² independently represent
H or C₁-C₂alkyl;
R³ and R⁴ independently represent H or C₁-C₃alkyl;
   X¹ represents O;
   X² represents O;
   W represents R⁶X¹—;
   R⁶ represents —C(═X¹)TZ;
   T represents O or a single bond;
   Z represents
C₁-C₁₈alkyl-heterocyclyl, [2′-(1H-tetrazol-5-yl)biphenyl-4-yl]methyl-heterocyclyl, [2′-(1H-tetrazol-5-yl)biphenyl-4-yl]methyl-heterocyclyl-methyl, [2′-(1H-tetrazol-5-yl)biphenyl-4-yl]methylamino-C₁-C₆alkyl, oxadiazolyl-biphenyl-methyl-heterocyclyl or heterocyclylmethyl-biphenyl, wherein said C₁-C₁₈alkyl-heterocyclyl, [2′-(1H-tetrazol-5-yl)biphenyl-4-yl]methyl-heterocyclyl, [2′-(1H-tetrazol-5-yl)biphenyl-4-yl]methyl-heterocyclyl-methyl, [2′-(1H-tetrazol-5-yl)biphenyl-4-yl]methylamino-C₁-C₆alkyl, oxadiazolyl-biphenyl-methyl-heterocyclyl or heterocyclylmethyl-biphenyl is optionally substituted by one or more substituents independently selected from halogen, OH, C₂-C₆alkanoyl, C₁-C₆alkyl, C₁-C₆alkoxy, heterocyclyloxy, hydroxyC₁-C₄alkyl and heterocyclyl;

### V—U—Y-M is

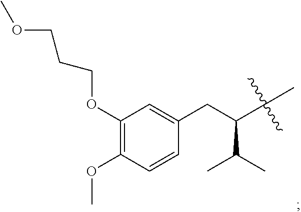 [SMILES: *[C@H](Cc1ccc(OC)c(OCCCOC)c1)C(C)C]

and

### A(R⁵)Q is

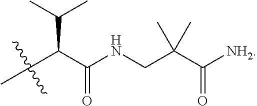 [SMILES: *[C@H](C(=O)NCC(C)(C)C(N)=O)C(C)C]

Specific compounds of the present invention are one or more of the following:
(4S,5S)-1-(isobutyryloxy)ethyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S)-pivaloyloxymethyl 5-[(5)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S)-isobutyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}-oxazolidine-3-carboxylate;
(4S,5S)-valyloxymethyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate, trifluoroacetic acid salt;
(4S,5S)-(ethoxycarbonyloxy)methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S)-(isopropoxycarbonyloxy)methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
{[(2S)-2-hydroxypropanoyl]oxy}methyl (4S,5S)-5-[(2S)-2-(3-amino-2,2-dimethyl-3-oxopropylaminocarbonyl)-3-methylbutyl]-4-{(2S)-2-[4-methoxy-3-(3-methoxypropoxy)benzyl]-3-methylbutyl}-1,3-oxazolidine-3-carboxylate;
{[(2S)-2-(ethoxymethoxy)propanoyl]oxy}methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate;
{(4S,5S)-5-[(2S)-2-(3-amino-2,2-dimethyl-3-oxopropylaminocarbonyl)-3-methylbutyl]-4-{(2S)-2-[4-methoxy-3-(3-methoxypropoxy)benzyl]-3-methylbutyl}-1,3-oxazolidin-3-yl-carbonyloxy}methyl morpholine-4-carboxylate;
(4S,5S) [(pyridine-3-yl)carbonyloxy]methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S) [(pyridine-2-yl)carbonyloxy]methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
[(2-methylpropoxycarbonyl)oxy]methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate;
{[(pyridin-3-ylmethoxy)carbonyl]oxy}methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate;
[(2-methyl-3-morpholin-4-ylpropanoyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
(1-methylpiperidine-4-carbonyloxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(1,3-dioxan-5-yl-oxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(1,3-dioxolan-4-ylmethoxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
[(3-hydroxy-2,2-dimethylpropanoyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[4-methoxybenzyloxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(benzyloxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
[(pyridine-4-yl)carbonyloxy]methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate;
{[(1-methyl-1H-imidazol-4-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxylpropoxy)benzyl]-3-methylbutyl}-1,3-oxazolidine-3-carboxylate;
[(1,3-dioxan-5-ylcarbonyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(1-methyl-1H-imidazol-5-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
({[(1-methyl-1H-imidazol-4-yl)methoxy]carbonyl}oxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
({[(1-methylpiperidin-4-yl)oxy]carbonyl}oxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-oxazolidine-3-carboxylate;
[(1-methylpiperidin-4-yl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
({[(1-methylpiperidin-4-yl)methoxy]carbonyl}oxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxylpropoxy)benzyl]-3-methylbutyl}-1,3-oxazolidine-3-carboxylate;
{[(1,3-dioxan-5-ylmethoxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxylpropoxy)benzyl]-3-methylbutyl}-1,3-oxazolidine-3-carboxylate;
(Pyridin-3-yloxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(dimethylamino)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(1-aminocyclopropyl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate, trifluoroacetate;
{[(1-methyl-1H-imidazol-2-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-{[(1-methyl-1H-imidazol-5-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-(1-aminocyclopropanecarbonyloxy)ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate, trifloroacetate;
1-({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methoxy)oxo-(2E)-but-2-enoic acid;
{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methyl 1-azabicyclo[2.2.1]heptane-4-carboxylate;
{1-[({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methoxy)carbonyl]cyclopropyl}methanaminium trifluoroacetate;
1-{[(1-methyl-1H-imidazol-4-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-{[(pyridin-3-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-{[(pyridin-2-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methoxy)-4-oxobutanoic acid;
1-({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}ethoxy)oxo-(2E)-but-2-enoic acid;
(1-methylpiperidin-4-yl)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(1-hydroxycyclopropyl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methyl N-pentanoyl-N-{[2′-(1H-tetrazol-5-yl)biphenyl-4-yl]methyl}-L-valinate;
(4S,5S)-ethyl 5-[(S)-2-(3-amino-2,2-dimethyl-3-oxopropylcarbamoyl)-3-methylbutyl]-4-{(S)-2-[4-methoxy-3-(3-methoxypropoxy)benzyl]-3-methylbutyl}-1,3-oxazolidine-3-carboxylate;
(4S,5S)-1-(isobutyryloxy)ethyl (4S,5S)-5-[(5)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S)-1-(isobutyryloxy)ethyl (4S,5S)-5-[(5)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S)—(N-CBz-valyloxy)methyl 5-[(5)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
{([2-methyl-2-(ethoxymethoxy)propanoyl]oxy}methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxylpropoxy)benzyl]-3-methylbutyl}-1,3-oxazolidine-3-carboxylate;
{[(3-methoxy-2,2-dimethyl-3-oxopropoxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
[({1-[(tert-butoxycarbonyl)amino]cyclopropyl}carbonyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-{1-[(tert-butoxycarbonyl)amino]cyclopropanecarbonyloxy}-ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methyl tert-butyl (2E)-but-2-enedioate;
{[(1-{[(tert-butoxycarbonyl)amino]methyl}cyclopropyl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}ethyl tert-butyl butanedioate;
1-{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}ethyl tert-butyl (2E)-but-2-enedioate;
1-{[(1-methyl-1H-imidazol-5-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-(Pyridin-3-yloxy)ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(2-Methylpyridin-3-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(3-Methylpyridin-2-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(4-Methyloxazol-5-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
({[1-(Hydroxymethyl)cyclopropyl]carbonyl}oxy)methyl (4S,5S)-5-(2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
Pyridine-3-ylmethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate; and
(4S,5S)-ethyl 5-[(5)-2-(3-amino-2,2-dimethyl-3-oxopropylcarbamoyl)-3-methylbutyl]-4-{(S)-2-[4-methoxy-3-(3-methoxypropoxy)benzyl]-3-methylbutyl}-2,2-dimethyloxazolidine-3-carboxylate;
   or a pharmaceutically accepted salt thereof.

In one embodiment of the present invention, there is provided a compound of formula (I)

wherein
the radical bonded to the nitrogen atom of the oxazolidine ring of the compound of formula (I) is selected from ethoxycarbonyl; isobutyryloxymethyloxycarbonyl; (chloromethyloxy)carbonyl; 1-(isobutyryloxy)ethyloxycarbonyl; (1-chloroethyloxy)carbonyl; pivaloyloxymethyloxycabonyl; isobutyloxycarbonyl, (N-boc-valyloxy)methyloxycarbonyl; valyloxymethyloxycarbonyl; (N-CBz-valyloxy)methyloxycarbonyl; (ethoxycarbonyloxy)methyloxycabonyl; (isopropoxycarbonyloxy); (Iodomethyloxy)carbonyl; {[(2S)-2-hydroxypropanoyl]oxy}methyloxycarbonyl; {[(2S)-2-(ethoxymethoxy)propanoyl]oxy}methyloxycarbonyl; [(Morpholine-4-carbonyloxy)methyloxy]carbonyl; (nicotinoyloxy)methyloxycarbonyl; (picolinoyloxy)methyloxycarbonyl; [(2-methylpropoxycarbonyl)oxy]methyloxycarbonyl; {[2-methyl-2-(ethoxymethoxy)propanoyl]oxy}methyloxycarbonyl; {[(pyridin-3-ylmethoxy)carbonyl]oxy}methyloxycarbonyl; [(2-methyl-3-morpholin-4-ylpropanoyl)oxy]methyloxycarbonyl; (1-methylpiperidine-4-carbonyloxy)methyloxycarbonyl; {[(1,3-dioxan-5-yl-oxy)carbonyl]oxy}methyloxycarbonyl; {[(1,3-dioxolan-4-ylmethoxy)carbonyl]oxy}methyloxycabonyl; [(3-hydroxy-2,2-dimethylpropanoyl)oxy]methyloxycabonyl; {[(4-methoxybenzyloxy)carbonyl]oxy}methyloxycarbonyl {[(benzyloxy)carbonyl]oxy}methyloxycarbonyl; (isonicotinoyloxy)methyloxycarbonyl {[(1-methyl-1H-imidazol-4-yl)carbonyl]oxy}methyloxycarbonyl; [(1,3-dioxan-5-ylcarbonyl)oxy]methyloxycarbonyl; {[(1-methyl-1H-imidazol-2-yl)carbonyl]oxy}methyloxycarbonyl; ({[(1-methyl-1H-imidazol-4-yl)methoxy]carbonyl}oxy)methyloxycarbonyl; ({[(1-methylpiperidin-4-yl)oxy]carbonyl}oxy)methyloxycarbonyl; [(1-methylpiperidin-4-yl)oxy]methyloxycarbonyl; {[(1-methylpiperidin-4-yl)methoxy]carbonyloxy}methyloxycarbonyl; {[(1,3-dioxan-5-yl)methoxy]carbonyloxy}methyloxycarbonyl; [(pyridin-3-yloxy)methyloxy]carbonyl {[(3-methoxy-2,2-dimethyl-3-oxopropoxy)carbonyl]oxy}methyloxycarbonyl; {[(dimethylamino)carbonyl]oxy}methyloxycarbonyl; [({1-[(tert-butoxycarbonyl)amino]cyclopropyl}carbonyl)oxy]methyloxycarbonyl; {[(1-aminocyclopropyl)carbonyl]oxy}methyloxycarbonyl; {[(1-methyl-1H-imidazol-5-yl)carbonyl]oxy}methyloxycarbonyl; {1-[(1-methyl-1H-imidazol-2-yl)carbonyloxy]ethyl}oxycarbonyl; 1-{[1-(N—BOC amino)-cyclopropane]carbonyloxy-ethyloxycarbonyl; {1-[(1-aminocyclopropane)carbonyloxy]-ethyl}oxycarbonyl; {[4-(t-butoxy)-4-oxo-(2E)-but-2-enoyl]oxy}methyloxycarbonyl; [(E)-(3-carboxy-prop-2-enoyl)oxy]methyloxycarbonyl; [(1-azabicyclo[2.2.1]heptane-4-carbonyloxy]methyloxycarbonyl; {[1-(N—BOC-amino)methyl-cyclopropyl]carbonyloxy}methyloxycarbonyl; {[1-(aminomethyl)cyclopropyl]carbonyloxy}methyloxycarbonyl; {1-[(1-methyl-1H-imidazol-4-yl)carbonyloxy]ethyloxy}carbonyl; {1-[(pyridin-3-yl)carbonyloxy]ethyloxy}carbonyl {1-[(pyridin-2-yl)carbonyloxy]ethyloxy}carbonyl; {[4-(t-butoxy)-4-oxo-butanoyl]oxy}methyloxycarbonyl; [(3-carboxy-propanoyl)oxy]methyloxycarbonyl; 1-{[4-(t-butoxy)-4-oxo-(2E)-but-2-enoyl]oxy}ethyloxycarbonyl; 1-[(E)-(3-carboxy-prop-2-enoyl)oxy]ethyloxycarbonyl; (1-methylpiperidin-4-yl)methyloxycarbonyl; {[(1-hydroxycyclopropyl)carbonyl]oxy}methyloxycarbonyl; 1-{[(1-methyl-1H-imidazol-5-yl)carbonyl]oxy}ethyloxycarbonyl; 1-(Pyridin-3-yloxy)ethyl]oxycarbonyl; {[(2-Methylpyridin-3-yl)carbonyl]oxy}methyloxycarbonyl; {[(3-Methylpyridin-2-yl)carbonyl]oxy}methyloxycarbonyl; {[1-(Hydroxymethyl)cyclopropyl]carbonyloxy}methyloxycabonyl; (Pyridine-3-yl)methyloxycarbonyl; (N-pentanoyl-N-[{2′-(1H-tetrazol-5-yl)biphenyl-4-yl]methyl}-L-valyloxy)methyloxycarbonyl; and {[(4-Methyloxazol-5-yl)carbonyloxy]methyloxy}carbonyl.

The scientific and technological terms and nomenclatures used hereinbefore and hereinafter have the same meaning as commonly understood by a person of ordinary skill in the art. In addition, the following definitions shall apply throughout the specification and the appended claims unless specifically stated otherwise:

The term “halogen” denotes fluoro, chloro, bromo and iodo groups.

In this document, the sign “-” is sometimes added to clarify which bond serves as a connection point. For example, heterocyclyl-C₁-Cₙalkyl represents a C₁-Cₙalkyl radical substituted with a heterocyclyl moiety, wherein C₁-Cₙalkyl and heterocyclyl are as defined below, where the heterocyclyl is bonded through the C₁-Cₙalkyl group. Further, RO-represents a radical wherein R is bonded to an oxygen atom and the said oxygen atom is at the connecting point for the whole radical.

The term “C₁-Cₙalkyl” denotes a straight or branched saturated alkyl group having 1 to n carbon atoms, wherein “n” is an integer from 1 to 18. Examples of “n” include 2, 3, 4, 5, 6, 7, 8 and 18. Examples of said alkyl include, but are not limited to, methyl, ethyl, propyl isopropyl, butyl, isobutyl, tert-butyl, pentyl, isopentyl and hexyl.

The term “C₂-Cₙalkenyl” denotes a straight or branched alkenyl group having saturated carbon-carbon bonds and at least one carbon-carbon double bond, and having 2 to n carbon atoms, wherein “n” is an integer from 2 to 18. Examples of “n” include 2, 3, 4, 5, 6, 7, 8 and 18. Examples of said alkenyl include, but are not limited to, ethenyl, 1-propenyl, 2-propenyl, isopropenyl and butenyl.

The term “C₂-Cₙalkynyl” denotes a straight or branched alkynyl group having saturated carbon-carbon bonds and at least one carbon-carbon triple bond, and having 2 to n carbon atoms, wherein “n” is an integer from 2 to 18. Examples of “n” include 2, 3, 4, 5, 6, 7, 8 and 18. Examples of said alkenyl include, but are not limited to ethynyl, propynyl and butynyl.

The term “C₃-Cₚcycloalkyl” denotes a saturated monocyclic ring having 3 to p carbon atoms, wherein p is an integer from 3 to 18. Examples of “p” include 2, 3, 4, 5, 6, 7, 8 and 18. Examples of said cycloalkyl include, but are not limited to cyclopropyl, cyclobutyl, cyclopentyl and cyclohexyl.

The term “C₃-Cₚcycloalkenyl” denotes a monocyclic ring having saturated carbon-carbon bonds and at least one carbon-carbon double bond, and having 3 to p carbon atoms, wherein p is an integer from 3 to 18. Examples of “p” include 2, 3, 4, 5, 6, 7, 8 and 18.

Examples of said cycloalkenyl include, but are not limited to, cyclobutenyl, cyclopentenyl and cyclohexenyl.

The term “C₄-Cₚcycloalkynyl” denotes a monocyclic ring having saturated carbon-carbon bonds and at least one carbon-carbon triple bond, and having 4 to p carbon atoms, wherein p is an integer from 3 to 18. Examples of “p” include 2, 3, 4, 5, 6, 7, 8 and 18. Examples of said cycloalkynyl include, but are not limited to, cyclobutynyl cyclopentynyl and cyclohexynyl

The term “C₁-Cₙalkoxy” denotes a C₁-Cₙalkyl as defined above linked to oxygen, i.e. C₁-Cₙalkyl-O. Examples of said alkoxy include, but are not limited to, methoxy, ethoxy, n-propoxy, isopropoxy and butyloxy.

The term “oxo” denotes a double-bonded oxygen atom. The oxo group may be attached to a carbon atom forming a carbonyl moiety or to a sulphur atom forming a sulphoxide moiety.

The term “C₃-Cₚcycloalkyl-C₁-Cₙalkyl” denotes a C₁-Cₙalkyl as defined above substituted with a C₃-Cₚcycloalkyl as defined above.

The term “C₃-Cₚcycloalkyl-C₂-Cₙalkenyl” denotes a C₂-Cₙalkenyl as defined above substituted with a C₃-Cₚcycloalkyl as defined above.

The term “C₃-Cₚcycloalkyl-C₂-Cₙalkynyl” denotes a C₂-Cₙalkynyl as defined above substituted with a C₃-Cₚcycloalkyl as defined above.

The term “aryl” denotes an aromatic ring or an aromatic ring fused with aromatic or non-aromatic carbocyclic or heterocyclic ring or rings forming a mono-, bi- or tricyclic ring system composed of 6-14 carbon atoms, preferably 6-10 carbon atoms. Examples of said aryl include, but are not limited to, phenyl, naphthyl, biphenyl, 2-naphthanyl, tetrahydronaphthyl, 2-indenyl, 4-indenyl and indanyl.

The term “aryl-C₁-Cₙalkyl” denotes a C₁-Cₙalkyl as defined above substituted with an aryl as defined above.

The term “aryl-C₂-Cₙalkenyl” denotes a C₂-Cₙalkenyl as defined above substituted with an aryl as defined above.

The term “aryl-C₂-Cₙalkynyl” denotes a C₂-Cₙalkynyl as defined above substituted with an aryl as defined above.

The term “heterocyclyl” denotes a saturated, partially unsaturated or aromatic mono-, bi- or tricyclic ring system composed of 4-18 atoms in which 1, 2, 3 or 4 of the atoms in the ring(s) is an element other than carbon independently selected from one or more of nitrogen, oxygen or sulphur. The term “nitrogen” shall be understood to include nitrogen oxide (NO). The term “sulphur” shall be understood to include “sulphoxide” (S(O)) and sulphone (SO₂). Examples of said heterocyclyl include, but are not limited to pyrrolidino, piperidino, oxetanyl, pyridinyl, piperazino, morpholino, dioxanyl, thiomorpholino, furanyl, tetrahydrofuranyl, pyranyl, tetrahydropyranyl, thiazolyl, oxazolyl, thiazinolyl, imidazolyl, triazolyl, tetrazolyl, isoxazolyl, oxadiazolyl, indolinyl, isoindolinyl, 2,3-dihydrobenzimidazolyl, 1,2,3,4-tetrahydroquinolyl, 1,2,3,4-tetrahydroisoquinolyl, 1,2,3,4-tetrahydro-1,3-benzodiazinyl, 1,2,3,4-tetrahydro-1,4-benzodiazinyl, 3,4-dihydro-2H-1,4-benzoxazinyl, 3,4-dihydro-2H-1,4-benzothiazinyl, 3,4-dihydro-2H-1,3-benzothiazinyl, 3,4,5,6,7,8-hexahydro-2H-1,4-benzoxazinyl, 3,4,5,6,7,8-hexahydro-2H-1,4-benzothiazinyl, 9-azabicyclo[3.3.1]non-9-yl, 1-azepan-1-yl, 2,8-diazaspiro[4.5]dec-8-yl, octahydroisoindol-2-yl, 3,7-diazabicyclo[3.3.1]non-3-yl, 3-azabicyclo[3.3.1]non-3-yl, 8-azabicyclo[3.2.1]oct-8-yl, 3-azabicyclo[3.2.2]non-3-yl, 5,6-dihydrophenanthridinyl, isoquinolinyl, tetrahydroisoquinolinyl, quinazolinyl, tetrahydroquinazolinyl, quinoxalinyl, benzimidazolyl, benzoxazolyl, benzisoxazolyl, benzothiazinolyl, benzisothiazinolyl, benzothiazolyl, benzoxadiazolyl, benzo-1,2,3-triazolyl, benzo-1,2,4-triazolyl, benzotetrazolyl, benzofuranyl, benzothienyl, benzopyridyl, benzopyrimidinyl, benzopyridazinyl, benzopyrazolyl, indolyl, isoindolyl indolinyl and isoindolinyl.

The term “heterocyclyl-C₁-Cₙalkyl” denotes a C₁-Cₙalkyl as defined above substituted with a heterocyclyl as defined above.

The term “heterocyclyl-C₂-Cₙalkenyl” denotes a C₂-Cₙalkenyl as defined above substituted with a heterocyclyl as defined above.

The term “heterocyclyl-C₂-Cₙalkynyl” denotes a C₂-Cₙalkynyl as defined above substituted with a heterocyclyl as defined above.

The radical tetrazolyl-biphenyl-methyl-heterocyclyl denotes heterocyclyl substituted with methyl, said methyl being substituted with biphenyl, said biphenyl being substituted with tetrazolyl. The same reasoning applies to tetrazolyl-biphenyl-methyl-heterocyclylmethyl, tetrazolyl-biphenyl-methyl-amino-C1-C6alkyl, oxadiazolyl-biphenyl-methyl-heterocyclyl and so on.

Unless otherwise indicated, the alkyl, alkenyl, alkynyl, alkoxy, cycloalkyl, cycloalkenyl, aryl and heterocyclyl (including those in composite expressions such as aryl-alkyl or heterocyclyl-alkyl) are independently optionally substituted with one or more substituents independently selected from halogen, hydroxyl, amino, oxo, mercapto, amido, cyano, azido, nitro, optionally substituted C₁-C₃alkyl, C₂-C₄alkenyl, C₂-C₄alkynyl, C₃-C₆cyclolkyl, C₁-C₄alkoxy, haloC₁-C₄alkyl, polyhaloC₁-C₄alkyl, hydroxyl-C₁-C₄alkyl, C₁-C₆alkylcarbonyl. It should be noted that the radical positions on any molecular moiety used in the definitions may be anywhere on such a moiety as long as it is chemically permitted and stable.

The term “optionally substituted” as used herein, means that substitution is optional, i.e. there may or may not be substitution. For instance, the expression “alkyl group optionally substituted with one or more substituents” means that the alkyl group is substituted by zero, one or more substituents.

The term “substituted” refers to a molecule wherein at least one hydrogen atom is replaced with a substituent.

Radicals used in the definitions of the variables include all possible isomers unless otherwise indicated. For instance pyridyl includes 2-pyridyl, 3-pyridyl and 4-pyridyl; pentyl includes 1-pentyl, 2-pentyl, 3-pentyl and the like. When any variable occurs more than one time in any constituent, each definition is independent.

Commonly used leaving groups, in the present invention also denoted L₁ and/or L₂, include, but are not limited to Cl, Br, I, sulfonates such as mesylate, brosylate, tosylate, triflate, nosylate, tresylate and the like.

While not wishing to be bound by any specific theory, it is believed that the compounds of the invention may serve as prodrugs. A prodrug may be defined as the temporary derivatization of one or several functional groups of a drug in such a manner as to release the drug in its active form after the administration (see for example Taylor, M., Advanced Drug Delivery Reviews 1996, 19: 131-148; Ettmayer, P., J. Med. Chem., 2004, 47, 2393).

The compounds of formulas (I) or their metabolites have activity as medicaments. In particular, the compounds of formula (I) or their metabolites may be renin inhibitors or prodrugs of renin inhibitors.

The compounds according to the present invention exhibit improved or enhanced properties compared to aliskiren or other renin inhibitors with respect to at least one of the following parameters: bioavailability, absorption, permeability through intestinal tract, permeability through skins, absorption by various drug administration routes, interaction with intestinal efflux system, drug-drug interaction, physico-chemical properties, pharmacokinetic properties, pharmacodynamic properties, such as t_{1/2}, tₘₐₓ, clearance, distribution, excretion, metabolic properties, during of action, interaction with Cytochrom p450 isozymes, properties for formulation, properties for production, inhibition of renin, inhibition of plasma renin activity, in vivo efficacy of renin activity inhibition, in vivo efficacy of treating or preventing hypertension, in vivo end-organ protection and properties in combination therapy with other medicines with cardiovascular effect.

Suitable tests for measurement of the above parameters include, but are not limited to, physico-chemical properties, stability in biological fluids, caco-2 permeability, PAMPA permeability (i.e. parallel artificial membrane permeability assay), interaction with efflux system, interaction with intestinal transporters, drug-drug interaction, interaction with CYP isozymes, permeation through skins, formulation properties for transdermal administration, formulation properties for various administration routes, in vivo pharmacokinetics in experimental animals via various administration routes, in vivo pharmacodynamics in experimental animal, in silico simulation of pharmacokinetic or pharmacodynamic properties, in silico simulation of physico-chemical properties, properties for drug delivery formulations, metabolism in liver extracts, metabolism in hepatocytes, safety properties, renin enzymatic assay, plasma renin activity, efficacy of treating or preventing hypertension in experimental animals, efficacy of end-organ protection in experimental animal, effect in combination therapy for hypertension or hypertension-related disorders and so on.

Certain compounds of the present invention may exist as tautomers or stereoisomers (e.g. racemate, enantiomer, diastereomer or E- or Z-isomer). It is to be understood that the present invention encompasses all such tautomers or stereoisomers.

Certain compounds of the present invention may exist as solvates or hydrates. It is to be understood that the present invention encompasses all such solvates or hydrates.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, nitric, methansulphonic, sulphuric, phosphoric, trifluoroacetic, para-toluene sulphonic, 2-mesitylen sulphonic, citric, acetic, tartaric, fumaric, lactic, succinic, malic, malonic, maleic, 1,2-ethanedisulphonic, adipic, aspartic, benzenesulphonic, benzoic, ethanesulphonic or nicotinic acid. In addition a suitable pharmaceutically acceptable salt of a compound of the invention, is, for example, a base-addition salt of a compound of the invention which is sufficiently acidic, for example, a metal salt, for example, sodium, potassium, calcium, magnesium, zinc or aluminum, an ammonium salt, a salt with an organic base which affords a physiologically acceptable cation, which includes quartery ammonium hydroxides, for example methylamine, ethylamine, diethylamine, trimethylamine, tert-butylamine, triethylamine, dibenzylamine, N,N-dibenzylethylamine, cyclohexylethylamine, tris-(2-hydroxyethyl)amine, hydroxyethyl diethylamine, (1R,2S)-2-hydroxyinden-1-amine, morpholine, N-methylpiperidine, N-ethylpiperidine, piperazine, methylpiperazine, adamantylamine, choline hydroxide, tetrabutylammonium hydroxide, tris-(hydroxymethyl)methylamine hydroxide, L-arginine, N-methyl D-glucamine, lysine or arginine.

The present invention also relates to a process for preparing a compound of formula (I), wherein R¹, R², R³ and R⁴ are H, said process comprising the steps of
a) reacting a compound of formula (II)

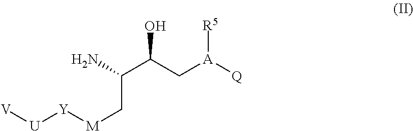

wherein M, Y, U, V, A, R⁵ and Q are as defined above, with a compound of formula (VIII),

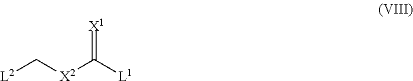

wherein X¹ and X² are as defined above and L¹ and L² are leaving groups independently selected from Cl, Br, I, sulfonates such as mesylate, brosylate, tosylate, triflate, nosylate and tresylate, under basic conditions in an inert solvent or mixture of inert solvents to obtain a compound of formula (IX)

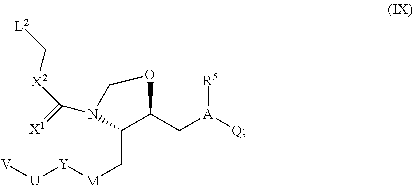

b) subsequently reacting the compound of formula (IX) with a compound of formula (X) or a salt thereof,

R⁶—OH (X)
wherein R⁶ is as defined in any previous claim, under basic conditions in an inert solvent or mixture of inert solvents.

The present invention also relates to a compound of general formula (IX)

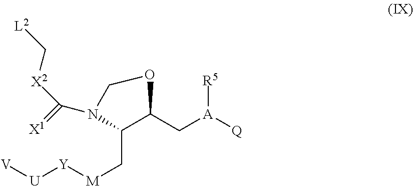

wherein X¹, X², M, Y, U, V, A, R⁵ and Q are as defined above and L² is as defined above.

### Pharmaceutical Preparations

The compounds of the present invention will normally be administrated via the oral, parenteral, intravenous, intramuscular, subcutaneous or other injectable ways, buccal, rectal, vaginal, transdermal and/or nasal route and/or via inhalation, in the form of pharmaceutical preparations comprising the active ingredient or a pharmaceutically acceptable salt or solvate thereof, or a solvate of such a salt, in a pharmaceutically acceptable dosage form. Depending upon the disorder and patient to be treated and the route of administration, the compositions may be administered at varying doses.

According to a further aspect of the invention there is provided a pharmaceutical composition including any of the compounds of the invention, or pharmaceutically acceptable derivatives thereof, in admixture with pharmaceutically acceptable excipients, oils which may be glycerides, diluents and/or carriers.

### Pharmacological Properties

The compounds of the formula (I) and their pharmaceutically usable salts, or metalated derivatives thereof (i.e. metal coordinated compounds or metal complexes), are renin inhibitors or prodrugs of renin inhibitors and may be used for the medication related to the inhibition of renin.

The compounds of the present invention may be used for the treatment of hypertension, congestive heart failure, cardiac hypertrophy, cardiac fibrosis, atherosclerosis; cardiac failure, cardiomyopathy, postinfarction, complications owing to diabetes such as nephropathy, vasculopathy and neuropathy, diseases of the cardiac vessels, restenosis after angioplasty, increased intraocular pressure, glaucoma, abnormal vascular growth, hyperaldosteronism, states of anxiety and cognitive disorders. The present invention further provides the use of a compound of formula (I) and pharmaceutically acceptable salts thereof in the treatment or prevention of hypertension and heart failure, and also glaucoma, cardiac infarction and kidney failure.

The present invention provides the use of a compound of formula (I) or a pharmaceutically acceptable salt thereof for the preparation of a medicament for the treatment and/or prophylaxis of hypertension, congestive heart failure, cardiac hypertrophy, cardiac fibrosis, atherosclerosis; cardiac failure, cardiomyopathy, postinfarction, complications owing to diabetes such as nephropathy, vasculopathy and neuropathy, diseases of the cardiac vessels, restenosis after angioplasty, increased intraocular pressure, glaucoma, abnormal vascular growth, hyperaldosteronism, states of anxiety and cognitive disorders, preferably hypertension.

In a further aspect, the present invention provides a compound of formula (I) or a pharmaceutically acceptable salt thereof for the treatment and/or prevention of hypertension, congestive heart failure, cardiac hypertrophy, cardiac fibrosis, atherosclerosis; cardiac failure, cardiomyopathy, postinfarction, complications owing to diabetes such as nephropathy, vasculopathy and neuropathy, diseases of the cardiac vessels, restenosis after angioplasty, increased intraocular pressure, glaucoma, abnormal vascular growth, hyperaldosteronism, states of anxiety and cognitive disorders, preferably hypertension.

Further, the present invention provides a method of treating and/or preventing hypertension, congestive heart failure, cardiac hypertrophy, cardiac fibrosis, atherosclerosis; cardiac failure, cardiomyopathy, postinfarction, complications owing to diabetes such as nephropathy, vasculopathy and neuropathy, diseases of the cardiac vessels, restenosis after angioplasty, increased intraocular pressure, glaucoma, abnormal vascular growth, hyperaldosteronism, states of anxiety and cognitive disorders comprising the administration of a therapeutically effective amount of a compound of formula (I) or a pharmaceutically acceptable salt thereof, to a mammal in need thereof.

The present invention provides the use of a compound of formula (I) or a pharmaceutically acceptable salt thereof for the preparation of a medicament for the treatment and/or prophylaxis of severe hypertension, pulmonary hypertension (PH), malignant hypertension, isolated systolic hypertension, familial dyslipidemic hypertension, high blood pressure, unstable coronary syndrome, ischaemic heart disease and ischaemic heart damage, myocardial infarction, unstable coronary syndrome, cardiac fibrosis, atherosclerosis, cardiomyopathy, vasculopathy, diastolic dysfunction, elevated total cholesterol, low LDL cholesterol, peripheral vascular disease (PVD), peripheral artery disease (PAD), peripheral venous disorders, coronary arterial disease (CAD), cerebrovascular diseases, metabolic disorder (Syndrome X), atrial fibrillation (AF), vascular inflammation, vasculitides or closure, aneurysm, angina, restenosis of dialysis access grafts, renal failure, renoprotection, complications owing to diabetes such as nephropathy, glomerulonephritis, nephrotic syndrome, renal fibrosis, acute interstitial nephritis (AIN), acute tubular nephritis (ATN), acute tubulo-interstitial nephritis, polycystic kidney disease (PKD), endothelial dysfunction and ormicroalbuminuria,

In a further aspect, the present invention provides a compound of formula (I) or a pharmaceutically acceptable salt thereof for the treatment and/or prevention of severe hypertension, pulmonary hypertension (PH), malignant hypertension, isolated systolic hypertension, familial dyslipidemic hypertension, high blood pressure, unstable coronary syndrome, ischaemic heart disease and ischaemic heart damage, myocardial infarction, unstable coronary syndrome, cardiac fibrosis, atherosclerosis, cardiomyopathy, vasculopathy, diastolic dysfunction, elevated total cholesterol, low LDL cholesterol, peripheral vascular disease (PVD), peripheral artery disease (PAD), peripheral venous disorders, coronary arterial disease (CAD), cerebrovascular diseases, metabolic disorder (Syndrome X), atrial fibrillation (AF), vascular inflammation, vasculitides or closure, aneurysm, angina, restenosis of dialysis access grafts, renal failure, renoprotection, complications owing to diabetes such as nephropathy, glomerulonephritis, nephrotic syndrome, renal fibrosis, acute interstitial nephritis (AIN), acute tubular nephritis (ATN), acute tubulo-interstitial nephritis, polycystic kidney disease (PKD), endothelial dysfunction and ormicroalbuminuria,

Further, the present invention provides a method of treating and/or preventing severe hypertension, pulmonary hypertension (PH), malignant hypertension, isolated systolic hypertension, familial dyslipidemic hypertension, high blood pressure, unstable coronary syndrome, ischaemic heart disease and ischaemic heart damage, myocardial infarction, unstable coronary syndrome, cardiac fibrosis, atherosclerosis, cardiomyopathy, vasculopathy, diastolic dysfunction, elevated total cholesterol, low LDL cholesterol, peripheral vascular disease (PVD), peripheral artery disease (PAD), peripheral venous disorders, coronary arterial disease (CAD), cerebrovascular diseases, metabolic disorder (Syndrome X), atrial fibrillation (AF), vascular inflammation, vasculitides or closure, aneurysm, angina, restenosis of dialysis access grafts, renal failure, renoprotection, complications owing to diabetes such as nephropathy, glomerulonephritis, nephrotic syndrome, renal fibrosis, acute interstitial nephritis (AIN), acute tubular nephritis (ATN), acute tubulo-interstitial nephritis, polycystic kidney disease (PKD), endothelial dysfunction and ormicroalbuminuria comprising the administration of a therapeutically effective amount a compound of formula (I) or a pharmaceutically acceptable salt thereof, to a mammal in need thereof.

The present invention relates to the use of a compound of formula (I) or a pharmaceutically acceptable salt thereof or a pharmaceutically acceptable salt thereof for the preparation of a medicament for the treatment and/or prophylaxis of hypertension, severe hypertension, pulmonary hypertension (PH), malignant hypertension, isolated systolic hypertension or familial dyslipidemic hypertension, heart failure, glaucoma, cardiac infarction, kidney failure, or restenosis.

The present invention relates to a method of treating and/or preventing hypertension, severe hypertension, pulmonary hypertension (PH), malignant hypertension, isolated systolic hypertension, familial dyslipidemic hypertension, comprising the administration of a therapeutically effective amount of a compound of formula (I) or a pharmaceutically acceptable salt thereof or claim hereinbefore or hereinafter or a pharmaceutically acceptable salt thereof to a mammal in need thereof.

The dose may vary within wide limits and has of course to be adapted to the individual circumstances in each individual case. In general, for oral administration, a daily dose of about 1 mg to about 2 g, preferably about 5 mg to about 1 g, per adult (assuming a weight of approximately 70 kg for the adult), divided into preferably 1-3 individual doses which may, for example, be of equal size, may be appropriate, although the upper limit specified may also be exceeded if this should be found to be appropriate; typically, children receive a lower dose according to their age and body weight.

### Combinations

The compounds of the present invention and the pharmaceutically usable salts thereof may also be administered in combination with one or more additional agents having cardiovascular action, for example α- and β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts.

A diuretic is, for example, a thiazide derivative selected from amiloride, chlorothiazide, hydrochlorothiazide, methylchlorothiazide, and chlorothalidon.

ACE inhibitors include alacepril, benazepril, benazapriiat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, azilsartan and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxentan, and tezosentan and their pharmaceutically acceptable salts.

The combination therapy includes co-administration of the compounds of the invention and said other agents, sequential administration of the compound and the other agents, administration of a composition containing the compound of the invention and the other agent, or simultaneous administration of separate compositions containing the compound and the other agent.

The present invention provides the use of a compound of formula (I) or a pharmaceutically acceptable salt thereof together with one or more of the compounds described in this combination section for the preparation of a medicament for the treatment and/or prophylaxis of hypertension, congestive heart failure, cardiac hypertrophy, cardiac fibrosis, atherosclerosis; cardiac failure, cardiomyopathy, postinfarction, complications owing to diabetes such as nephropathy, vasculopathy and neuropathy, diseases of the cardiac vessels, restenosis after angioplasty, increased intraocular pressure, glaucoma, abnormal vascular growth, hyperaldosteronism, states of anxiety and cognitive disorders, preferably hypertension.

In a further aspect, the present invention provides a compound of formula (I) or a pharmaceutically acceptable salt thereof together with one or more of the compounds described in this combination section for the treatment and/or prevention of hypertension, congestive heart failure, cardiac hypertrophy, cardiac fibrosis, atherosclerosis; cardiac failure, cardiomyopathy, postinfarction, complications owing to diabetes such as nephropathy, vasculopathy and neuropathy, diseases of the cardiac vessels, restenosis after angioplasty, increased intraocular pressure, glaucoma, abnormal vascular growth, hyperaldosteronism, states of anxiety and cognitive disorders, preferably hypertension.

Further, the present invention provides a method of treating and/or preventing hypertension, congestive heart failure, cardiac hypertrophy, cardiac fibrosis, atherosclerosis; cardiac failure, cardiomyopathy, postinfarction, complications owing to diabetes such as nephropathy, vasculopathy and neuropathy, diseases of the cardiac vessels, restenosis after angioplasty, increased intraocular pressure, glaucoma, abnormal vascular growth, hyperaldosteronism, states of anxiety and cognitive disorders comprising the administration of a therapeutically effective amount of a compound of formula (I) or a pharmaceutically acceptable salt thereof together with one or more of the compounds described in this combination section, to a mammal in need thereof.

The present invention provides the use of a compound of formula (I) or a pharmaceutically acceptable salt thereof together with one or more of the compounds described in this combination section for the preparation of a medicament for the treatment and/or prophylaxis of severe hypertension, pulmonary hypertension (PH), malignant hypertension, isolated systolic hypertension, familial dyslipidemic hypertension, high blood pressure, unstable coronary syndrome, ischaemic heart disease and ischaemic heart damage, myocardial infarction, unstable coronary syndrome, cardiac fibrosis, atherosclerosis, cardiomyopathy, vasculopathy, diastolic dysfunction, elevated total cholesterol, low LDL cholesterol, peripheral vascular disease (PVD), peripheral artery disease (PAD), peripheral venous disorders, coronary arterial disease (CAD), cerebrovascular diseases, metabolic disorder (Syndrome X), atrial fibrillation (AF), vascular inflammation, vasculitides or closure, aneurysm, angina, restenosis of dialysis access grafts, renal failure, renoprotection, complications owing to diabetes such as nephropathy, glomerulonephritis, nephrotic syndrome, renal fibrosis, acute interstitial nephritis (AIN), acute tubular nephritis (ATN), acute tubulo-interstitial nephritis, polycystic kidney disease (PKD), endothelial dysfunction and ormicroalbuminuria.

In a further aspect, the present invention provides a compound of formula (I) or a pharmaceutically acceptable salt thereof together with one or more of the compounds described in this combination section for the treatment and/or prevention of severe hypertension, pulmonary hypertension (PH), malignant hypertension, isolated systolic hypertension, familial dyslipidemic hypertension, high blood pressure, unstable coronary syndrome, ischaemic heart disease and ischaemic heart damage, myocardial infarction, unstable coronary syndrome, cardiac fibrosis, atherosclerosis, cardiomyopathy, vasculopathy, diastolic dysfunction, elevated total cholesterol, low LDL cholesterol, peripheral vascular disease (PVD), peripheral artery disease (PAD), peripheral venous disorders, coronary arterial disease (CAD), cerebrovascular diseases, metabolic disorder (Syndrome X), atrial fibrillation (AF), vascular inflammation, vasculitides or closure, aneurysm, angina, restenosis of dialysis access grafts, renal failure, renoprotection, complications owing to diabetes such as nephropathy, glomerulonephritis, nephrotic syndrome, renal fibrosis, acute interstitial nephritis (AIN), acute tubular nephritis (ATN), acute tubulo-interstitial nephritis, polycystic kidney disease (PKD), endothelial dysfunction and ormicroalburninuria.

Further, the present invention provides a method of treating and/or preventing severe hypertension, pulmonary hypertension (PH), malignant hypertension, isolated systolic hypertension, familial dyslipidemic hypertension, high blood pressure, unstable coronary syndrome, ischaemic heart disease and ischaemic heart damage, myocardial infarction, unstable coronary syndrome, cardiac fibrosis, atherosclerosis, cardiomyopathy, vasculopathy, diastolic dysfunction, elevated total cholesterol, low LDL cholesterol, peripheral vascular disease (PVD), peripheral artery disease (PAD), peripheral venous disorders, coronary arterial disease (CAD), cerebrovascular diseases, metabolic disorder (Syndrome X), atrial fibrillation (AF), vascular inflammation, vasculitides or closure, aneurysm, angina, restenosis of dialysis access grafts, renal failure, renoprotection, complications owing to diabetes such as nephropathy, glomerulonephritis, nephrotic syndrome, renal fibrosis, acute interstitial nephritis (AIN), acute tubular nephritis (ATN), acute tubulo-interstitial nephritis, polycystic kidney disease (PKD), endothelial dysfunction and ormicroalbuminuria comprising the administration of a therapeutically effective amount of a compound of formula (I) or a pharmaceutically acceptable salt thereof together with one or more of the compounds described in this combination section, to a mammal in need thereof.

The present invention provides the use of a compound of formula (I) or a pharmaceutically acceptable salt thereof together with one or more of the compounds described in this combination section for the preparation of a medicament for the treatment and/or prophylaxis of hypertension, severe hypertension, pulmonary hypertension (PH), malignant hypertension, isolated systolic hypertension or familial dyslipidemic hypertension.

The present invention provides a method of treating and/or preventing hypertension, severe hypertension, pulmonary hypertension (PH), malignant hypertension, isolated systolic hypertension, familial dyslipidemic hypertension, comprising the administration of a therapeutically effective amount of a compound of formula (I) or a pharmaceutically acceptable salt thereof together with one or more of the compounds described in this combination section, to a mammal in need thereof.

### Methods of Preparation

The compounds of the present invention may be prepared as outlined in the Schemes below. However, the invention is not limited to these methods. The compounds may also be prepared as described for structurally related compounds in the prior art. The reactions can be carried out according to standard procedures or as described in the experimental section. Hence, the compounds of the present invention may be prepared by any of the applicable methods and techniques of organic synthesis known to the skilled person.

In the course of the process described below for the preparation of compounds of formula (I), functional groups in starting materials which are prone to participate in undesired side reactions, especially amino, carboxy, hydroxy, and mercapto groups, may be protected by suitable conventional protecting groups which are customarily used in the organic synthesis. Those protecting groups may already be present in the precursors and are intended to protect the functional groups in question against undesired secondary reactions, such as acylation, etherification, esterification, oxidation, solvolysis, etc. In certain cases the protecting groups can additionally cause the reactions to proceed selectively, for example stereoselectively. It is characteristic of protecting groups that they can be removed easily, i.e. without undesired secondary reactions taking place, for example by acid treatment, fluoride treatment, solvolysis, reduction, or by photolysis. Protecting groups may also be present in the end products. Compounds of formula (I) having protected functional groups may have greater metabolic stability or pharmacodynamic properties that are better in some other way than the corresponding compounds having free functional groups. The protection of functional groups by such protecting groups, the protecting groups themselves, and the reactions for their removal are described in standard works.

Schemes 1-5 illustrate different processes for synthesizing compounds of formula (I) or a compound which may be converted to a compound of formula (I).

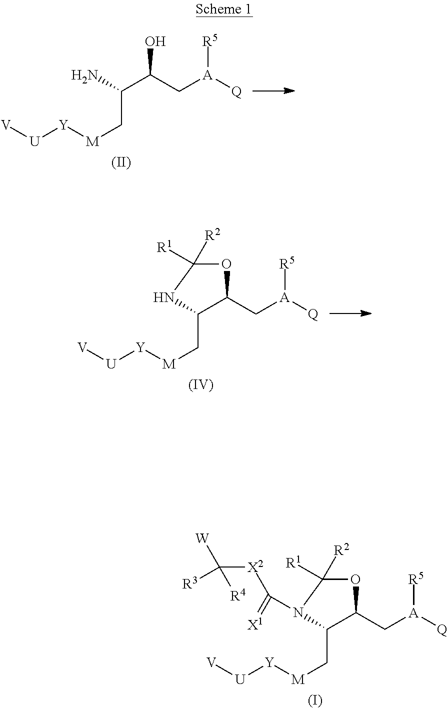

Scheme 1 describes a method of preparation of compounds according to formula (I), wherein R¹, R², R³, R⁴, X¹, X², W, M, Y, U, V, A, R⁵ and Q are as defined above hereinbefore or hereinafter. The vicinal aminoalcohol of formula (II), which may be a renin inhibitor, is reacted with a reagent containing R¹ and R² groups such as common aldehydes, ketones or dialkylacetals which include, for example, formaldehyde, dimethoxymethane, acetone, acetaldehyde, 1,1-dimethoxyethane and 2,2-dimethoxypropane, cyclopropanone, cyclobutanone, cyclopentanone, cyclohexanone, 2-methoxypropene-1 and the like. Solvents, if used, for the reaction may either be a single inert solvent or a mixture of inert solvents, such as dichloromethane, chloroform, acetonitrile, THF, 1,4-dioxane, DMF, benzene, toluene, 2,6-lutidine or acetone. The reaction may require dehydrating agents, such as molecular sieves and/or other water binding materials, or conditions. The reaction may be performed at room temperature or at elevated temperatures. An acid catalyst may be needed for the reaction. Commonly used catalysts for the reaction include organic or inorganic acids such as sulfonic acids, trifluoroacetic acid, Lewis acids, hydrochloric acids and the like. In some cases, the compound of formula (IV) may be prepared by reacting the amino and hydroxyl group of formula (II) with polymerized aldehydes, for example paraformaldehyde. In the reaction with formaldehyde, the product obtained may be a mixture of a monomer and a dimers with methylene bridge connecting the two oxazolidine rings as reported in the literature (Salos-Coronado R. et al, Heterocycles, 60, 2003, 1118). The compound of formula (IV) may be subsequently acylated with the desired acylating agents. The N-acylation reaction may be performed by using activated acylating reagents or by the addition of coupling agents using the typical procedures in chemical literature. Wherever needed, the desired acylating reagent should be properly protected by the appropriate protecting group. The commonly used activated forms of the acylating agents include, but are not limited to, alkoxycarbonyl chloride, alkoxycarbonyl bromide, alkoxythiocarbonyl chloride, and their appropriate derivatives. The reaction may be performed in the presence of a base, such as triethylamine, DIPEA, DMAP, potassium carbonate, sodium carbonate, cesium carbonate, DBU, pyridines, or other organic or inorganic bases suitable for such reactions. Commonly used solvents for the reaction include dichloromethane, dichloroethane, chloroform, THF, DMF, 1,4-dioxane, acetonitrile and other common solvents suitable for acylation reaction. The reaction may be performed at room temperature or elevated temperature. The reaction process may be monitored by LCMS, TLC and/or other methods. The products are isolated using common purification methods, such as column chromatography, crystallization or

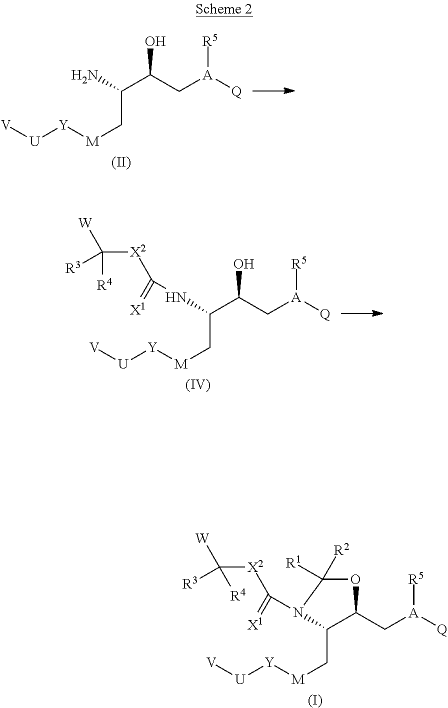

Scheme 2 describes a method of preparation of compounds according to formula (I), wherein R¹, R², R³, R⁴, X², W, M, Y, U, V, A, R⁵ and Q are as defined above. The amino group of the compound of formula (II), which may be a renin inhibitor, is reacted with an appropriate reagent, for example alkoxycarbonyl chloride, to form an N-acylated intermediate of formula (III) which may subsequently react with various cyclization agent such as aldehyde/ketone like formaldehyde, acetaldehyde, acetone or acetal/ketal like 2,2-dimethoxypropane, 1,1-dimethoxyethane in the presence of acidic catalysts like p-toluenesulfonic acid or Lewis acids, in particular boron trifluoride etherate, to afford a compound of formula (I). (See, for instance, Vidyasagar Reddy G et al, Tetrahedron Lett., 2000, 41, 949-51 and Jian-kang J. et al, J. Med. Chem., 51, 2008, 8012-8.)

It is understood that some compounds of formula (I) may be further modified to obtain desired compounds which can also be represented by formula (I). The methods for such modifications depend on the structures of the desired products and the structure of the compound of formula (I). Such modification reaction may involve deprotection, substitution, addition, oxidation, reduction and other chemical transformations which are common in organic syntheses.

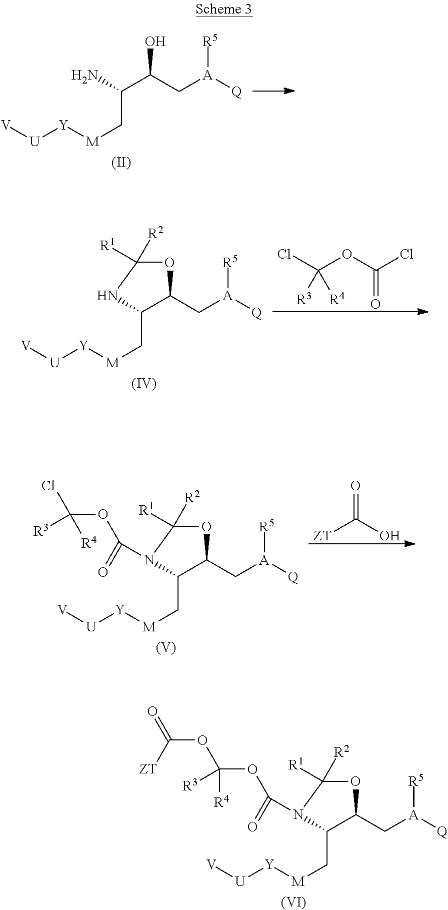

Scheme 3 describes a method of preparation for a compound of formula (I) wherein W is ZTC(O)O, R¹, R², R³, R⁴, M, m Y, U, V, Z, T, A, R⁵ and Q are as defined above, herein named compound (VI). The amino and hydroxyl group of formula (II) may be converted to a compound of formula (IV) as shown in Scheme 1. The compound of formula (IV) may be subsequently converted to a compound of formula (V) by reaction with a 1-haloalkyloxycarbonyl halide, for example chloromethyl chloroformate, 1-chloroethyl chloroformate. The chlorine of the compound of formula (V) may be substituted by an appropriate carboxylic acid, its salt or a salt of carbonate monoester. Alternatively, the chloromethyl group of the compound of formula (V) may be converted to bromo- or iodomethyl group, which is then followed by the substitution reaction. The conversion to bromomethyl group or iodomethyl can be performed in situ by standard procedures known to chemists skilled in the art. The compound of formula (V) may be reacted with a carboxylic acid, its salt or a salt of carbonate monoester to afford a compound of formula (I) wherein W is ZTC(O)O—. The reaction may be performed at room temperature or elevated temperature in inert solvents, such as dichloromethane, 1,2-dichloroethane, acetonitrile, THF, DMF, NMP or other suitable solvents. The reaction can be performed with the addition of appropriate bases, for example triethylamine, DIPEA, DMAP, potassium carbonate, sodium carbonate, cesium carbonate, silver carbonate, tetra-n-butylammonium hydroxide or other suitable organic or inorganic bases. The carboxylic acids include for example aliphatic carboxylic acids, aromatic carboxylic acids, heterocyclic containing aliphatic carboxylic acids and so on. The salts of carbonate monoesters include for example cesium salts of carbonate alkyl monoester, carbonate aryl monoester, carbonate heterocyclyl-containing-alkyl monoester. When carboxylic acids, carboxylic acid salts or salts of carbonate monoester are used, they are containing functional groups which are prone to participate in undesired side reactions, especially amino, carboxy, hydroxy, and mercapto groups, these functional groups may be protected by suitable conventional protecting groups, which are customarily used in organic synthesis.

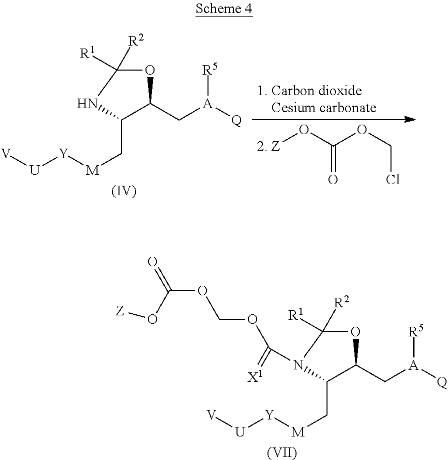

Scheme 4 describes an alternative method of preparation for a compound of formula (I) wherein W is ZOC(O)O, both R³ and R⁴ are H, R¹, R², X¹, M, Y, U, V, A, R⁵ and Q are as defined above, herein named compound of formula (VII). Z is preferably a C₁-C₆alkyl group as described hereinbefore or hereinafter. The oxazolidine of formula (IV) may be prepared as described above, and subsequently reacted with carbon dioxide in the presence of cesium carbonate followed by reaction with an appropriate reagent such as (alkyloxy)carbonyloxy-chloromethane. This may be a one-pot reaction. Suitable solvents for the reaction include DMF and THF. The product of formula (VII) may be isolated by purification methods known to persons skilled in the art.

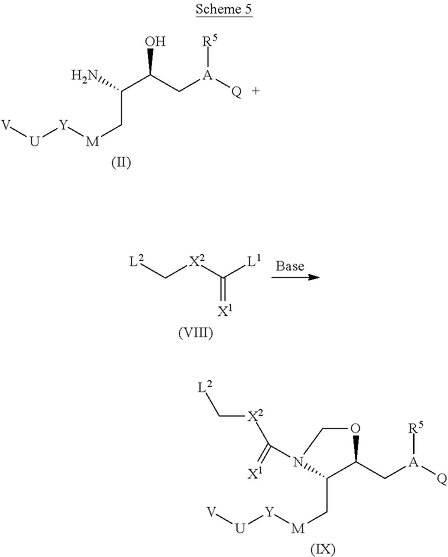

Scheme 5 describes a method of preparation of compounds of formula (IX), which may be further converted to compounds of formula (I). The amino group and hydroxyl group of a compound of formula (II) may be reacted with a compound of formula (VIII) to form a compound of formula (IX). In scheme 5, M, Y, U, V, A, R⁵, Q, X¹ and X² are as defined above. L¹ and L² are leaving groups, such as Cl, Br, I, sulfonates such as mesylate, brosylate, tosylate, triflate, nosylate and tresylate. The reaction may be performed in the presence of a base such as sodium carbonate, potassium carbonate or cesium carbonate. The compound of formula (VIII) may be chloromethyl chloroformate.

It is to be understood that the method of preparation described in Scheme 5 is not limited to amino alcohols of formula (II), but may be performed with other vicinal amino alcohols.

### EXAMPLES

The present invention is illustrated, but not limited, by the following Examples.

### ABBREVIATIONS

### DIPEA N; N-diisopropylethylamine;

DMAP 4-dimethylaminopyridine;
DBU 2,3,4,6,7,8,9,10-octahydropyrimidol[1,2-a]azepine;
EtOAc ethyl acetate;
Et₃N triethylamine;
THF tetrahydrofuran;

### DMF N,N-dimethylformamide;

DCM dichloromethane;
iPrOH isopropanol;
LCMS liquid chromatography mass spectroscopy;
TLC thin layer chromatography;
TFA trifluoroacetic acid;

### NMP N-methylpyrrolidone;

NMR nuclear magnetic resonance;
IR infrared spectroscopy;
MS mass spectrometry;
h hour(s);
min. or min minute(s);
ng nanogram;
ml milliliter;
poor p.o per oral;
AUC area under the curve;
Rpm. Rotation per minute;
i.v. or i v intravenous.

### GENERAL EXPERIMENTAL PROCEDURES

All evaporations are performed under reduced pressure, preferably between 2 and 100 mmHg. All temperatures are reported in Celsius degree. Unless stated otherwise, the reaction takes place at room temperature. In general, abbreviations used are those conventional in the art. The structure of final products and intermediates is confirmed by standard analytical methods, e.g. melting points, LC/MS (Agilent 1200/6120 system), NMR (Jeol 500 MHz NMR Spectrometer)).

The naming of the compounds in this document was made using the program “ChemOffice Pro Version 11” provided by Cambridge Scientific Computing Inc. If there is any inconsistency between the chemical name of the exemplified chemical compound and corresponding structure of said example, then the chemical structure should be used for determining the chemical compound of said example.

### Example 1

### (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine

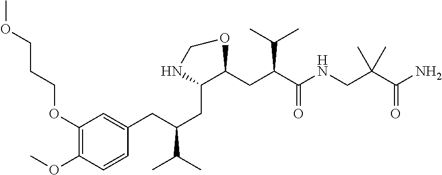 [SMILES: COCCCOc1cc(C[C@@H](C[C@@H]2NCO[C@H]2C[C@H](C(=O)NCC(C)(C)C(N)=O)C(C)C)C(C)C)ccc1OC]

To a solution of Aliskiren (free base, 100 mg) in THF (3 ml), which is cooled in ice bath, formaline (equimolar amount of 37% water solution of paraformaldehyde, 15 μl) was added and the reaction was kept under stirring for 5 h at cooling in an ice bath. To the reaction was then added water and dichloromethane. After extraction, the dichlomethane layer was collected and washed with brine and dried over magnesium sulfate. The solution was filtered and concentrated by rotary evaporation to give (102 mg) of white foam. MS: 564 [M+1]⁺, 587 [M+Na]⁺

### Example 2

### (4S,5S)-ethyl 5-[(S)-2-(3-amino-2,2-dimethyl-3-oxopropylcarbamoyl)-3-methylbutyl]-4-{(S)-2-[4-methoxy-3-(3-methoxypropoxy)benzyl]-3-methylbutyl}-1,3-oxazolidine-3-carboxylate

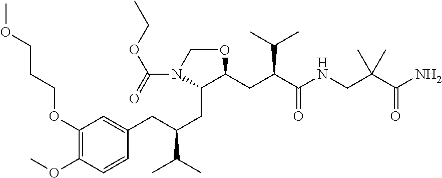 [SMILES: CCOC(=O)N1CO[C@@H](C[C@H](C(=O)NCC(C)(C)C(N)=O)C(C)C)[C@@H]1C[C@H](Cc1ccc(OC)c(OCCCOC)c1)C(C)C]

To a solution of (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine (90 mg, crude) and DMAP (29 mg, 0.2 mM) in 10 ml dry DCM under nitrogen atmosphere, ethylchloroformate (0.05 ml, 0.4 mM) was added by syringe and the solution was stirred for 48 h at room temperature. Reaction mixture was concentrated by rotary evaporation and purified by column chromatography on silica (THF/hexane 4:6, 5% of iPrOH) to give the product as colorless oil 24 mg. MS: 636 [M+1]⁺

### Example 3

### (4S,5S)-ethyl 5-[(S)-2-(3-amino-2,2-dimethyl-3-oxopropylcarbamoyl)-3-methylbutyl]-4-{(S)-2-[4-methoxy-3-(3-methoxypropoxy)benzyl]-3-methylbutyl}-2,2-dimethyloxazolidine-3-carboxylate

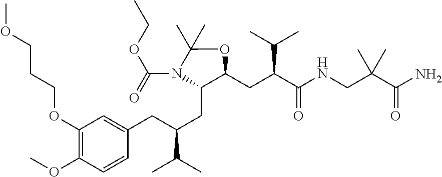 [SMILES: CCOC(=O)N1[C@@H](C[C@H](Cc2ccc(OC)c(OCCCOC)c2)C(C)C)[C@H](C[C@H](C(=O)NCC(C)(C)C(N)=O)C(C)C)OC1(C)C]

### Step a

Ethyl (1S,2S,4S)-4-{[(3-amino-2,2-dimethyl-3-oxopropyl)amino]carbonyl}-2-hydroxy-1-{(2S)-2-[4-methoxy-3-(3-methoxypropoxy)benzyl]-3-methylbutyl}-5-methylhexyl)carbamate

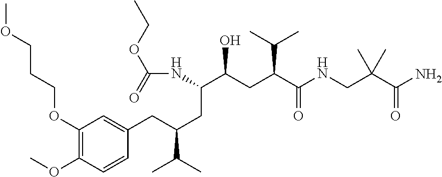 [SMILES: CCOC(=O)N[C@@H](C[C@H](Cc1ccc(OC)c(OCCCOC)c1)C(C)C)[C@@H](O)C[C@H](C(=O)NCC(C)(C)C(N)=O)C(C)C]

To a suspension of Aliskiren (hemifumarate, 200 mg) and sodium carbonate in 10 ml water, ethyl chlorofommte (0.2 ml) was added dropwise at room temperature. The reaction mixture was stirred overnight and extracted with DCM. The organic layer was dried over MgSO₄, filtered and concentrated by rotary evaporation. The product was isolated by column chromatography on silica (EtOAc, 2% MeOH) to give 190 mg. MS: 624 [M+1]⁺. 646 [M+Na]⁺

### Step b

Ethyl (1S,2S,4S)-4-{[(3-amino-2,2-dimethyl-3-oxopropyl)amino]carbonyl}-2-hydroxy-1-{(2S)-2-[4-methoxy-3-(3-methoxypropoxy)benzyl]-3-methylbutyl}-5-methylhexyl)carbamate (140 mg, 0.2 mM) was dissolved in dimethoxypropane (7 ml) and acetone (3 ml) and boron trifluoride etherate (2 drops) was added until a dark red colour persisted. The solution was stirred for 1.5 h at room temperature and then quenched with Et₃N. The solvent was removed by rotary evaporation and the product was isolated by column chromatography on silica gel (THF/hexane 4:6, 5% of iPrOH) to give the product 116 mg. MS: 665 [M+1]⁺

### Example 4

### (4S,5S)-1-(isobutyryloxy)methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate

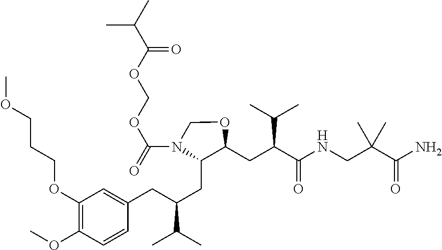 [SMILES: COCCCOc1cc(C[C@@H](C[C@H]2[C@H](C[C@H](C(=O)NCC(C)(C)C(N)=O)C(C)C)OCN2C(=O)OCOC(=O)C(C)C)C(C)C)ccc1OC]

### Method A:

### Step a

Chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate.

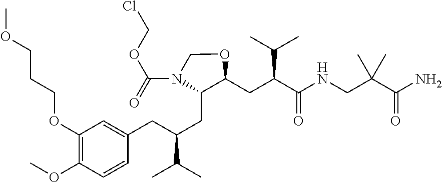 [SMILES: COCCCOc1cc(C[C@@H](C[C@H]2[C@H](C[C@H](C(=O)NCC(C)(C)C(N)=O)C(C)C)OCN2C(=O)OCCl)C(C)C)ccc1OC]

To a suspension of Aliskiren (hemifumarate, 100 mg) and sodium carbonate (100 mg) in water (15 ml), chloromethylchloroformate (0.15 ml) was added dropwise at room temperature. After 40 min of stirring, a sticky mass was formed and dichloromethane (10 ml) was added and reaction was carried out in the bi-phasic system. The reaction mixture was stirred overnight and extracted with more dichloromethane. The organic layer was dried over MgSO₄, filtered and concentrated by rotary evaporation to get product as an oil which was purified by column chromatography on silica to give desired compound as an oil (37 mg). MS: 656 [M+1]⁺

### Step b

Chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (37 mg) and isobutyric acid (25 mg) were dissolved in dry dichloromethane and diisopropylethylamine (30 μl) was added to the reaction mixture. Reaction mixture was under reflux (reflux) for 2 days. The reaction was monitored by LC-MS. To the reaction mixture was then added 3 ml 10% aqueous solution of citric acid and extracted into DCM. The organic layer washed with brine and dried over magnesium sulfate. After concentration under reduced pressure, the oily residue was purified by column chromatography on silica gel (THF/petroleum ether 4:6 with 5% iPrOH) to give the product (15 mg). MS: 708 [M+1]⁺. 730 [M+Na]⁺

### Method B:

To a solution of (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine (100 mg, prepared using the method described in Example 1) and DMAP (32 mg, 0.2 mM) in 15 ml dry dichloromethane under nitrogen atmosphere, (chlorocarbonyloxy)methyl isobutyrate (0.05 ml) prepared using a literature method was added by syringe and the solution was stirred for 24 h at room temperature. Reaction mixture was washed with water, and aqueous phase was washed with DCM (3×20 ml). Combined organic phases were washed with brine and dried over MgSO₄, filtered and concentrated by rotary evaporation. Crude product was purified by column chromatography on silica to give 26 mg product. MS: 708 [M+1]⁺, 730 [M+Na]⁺

### Example 5

### (4S,5S)-1-(isobutyryloxy)ethyl (4S,5S)-5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate

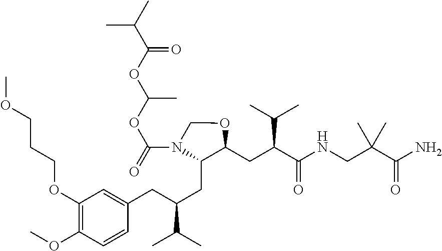 [SMILES: COCCCOc1cc(C[C@@H](C[C@H]2[C@H](C[C@H](C(=O)NCC(C)(C)C(N)=O)C(C)C)OCN2C(=O)OC(C)OC(=O)C(C)C)C(C)C)ccc1OC]

### Step a

1-Chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-oxazolidine-3-carboxylate.

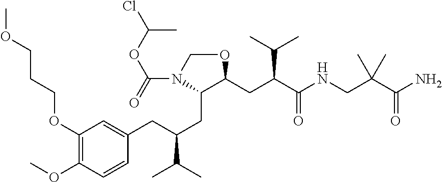 [SMILES: COCCCOc1cc(C[C@@H](C[C@H]2[C@H](C[C@H](C(=O)NCC(C)(C)C(N)=O)C(C)C)OCN2C(=O)OC(C)Cl)C(C)C)ccc1OC]

To a solution of (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine (117 mg, prepared using the method described in Example 1) and DMAP (47 mg) in dry dichloromethane (15 ml) under nitrogen atmosphere, 1-chloroethylchloroformate (35 μl) was added by syringe. The reaction mixture was stirred at room temperature overnight. The reaction mixture was diluted with dichloromethane, and then washed with 10% aqueous solution of citric acid, and brine. The organic phase was dried over magnesium sulfate, concentrated in vacuo. The residue was purified by column chromatography on silica gel (THF/petroleum ether 4:6+5% of iPrOH) to give 100 mg of coloureless oil as main fraction. The intermediate was used directly for next step.

### Step b

The above intermediate (100 mg) and isobutyric acid (40 mg) and DIPEA (80 μl) were dissolved in 10 ml TI-IF. The reaction mixture was stirred at 60° C. for 16 h. The reaction was monitored using LC-MS. An additional portion of butyric acid (40 mg) and DIPEA (80 μl) was added and the reaction was under stirring at kept at 60° C. for 40 h. After starting material peak disappearing from LC-MS analysis, 10 ml 10% aqueous solution of citric acid reaction mixture was added and the reaction mixture was extracted with dichloromethane. The collected organic phase was washed with brine and dried over magnesium sulfate. After evaporation in vacuo, the oily residue oil was purified by column chromatography on silica gel (THF/petroleum ether 4:6 with 5% iPrOH) to give 70 mg product. MS: 722 [M+1]⁺, 744 [M+Na]⁺

### Example 6

### (4S,5S)-pivaloyloxymethyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate

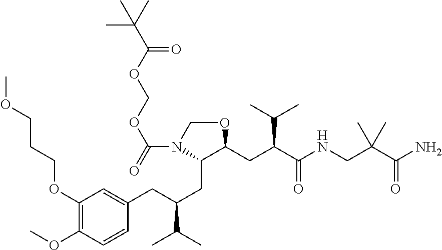 [SMILES: COCCCOc1cc(C[C@@H](C[C@H]2[C@H](C[C@H](C(=O)NCC(C)(C)C(N)=O)C(C)C)OCN2C(=O)OCOC(=O)C(C)(C)C)C(C)C)ccc1OC]

Chloromethyl (4S,5S)-5-((2S)-2-{[(3-amino-2,2-dimethyl-3-oxopropyl)amino]carbonyl}-3-methylbutyl)-4-((2S)-2-[4-methoxy-3-(3-methoxypropoxy)benzyl]-3-methylbutyl-1,3-oxazolidine-3-carboxylate (97 mg, prepared using the method described in Example 4, Method A), pivalic acid (160 mg) and diisopropylethylamine (0.27 ml) were dissolved in 10 ml dry THF and reaction mixture was stirred at 75° C. for 48 h. The reaction was monitored using LC-MS. After starting material peak had disappeared for the LCMS analysis, the 15 ml 10% aqueous solution of citric acid was added to the reaction mixture, and the reaction mixture was then extracted with dichlormethane. The organic layer was collected and washed with brine and dried over magnesium sulfate. After evaporation in vacuo, the oily residue was purified by column chromatography on silica gel (EtOAc) to give 15 mg product. MS: 722 [M+1]⁺, 744 [M+Na]⁺

### Example 7

### (4S,5S)-isobutyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}-oxazolidine-3-carboxylate

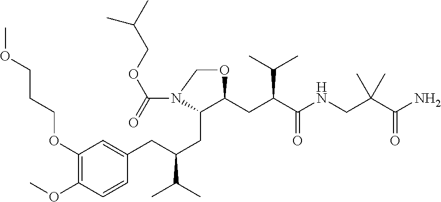 [SMILES: COCCCOc1cc(C[C@@H](C[C@H]2[C@H](C[C@H](C(=O)NCC(C)(C)C(N)=O)C(C)C)OCN2C(=O)OCC(C)C)C(C)C)ccc1OC]

To a solution of (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine (200 mg, 0.4 mM) and DMAP (70 mg, 0.6 mM) in dry dichloromethane under nitrogen atmosphere, isobutylchloroformate (55 mg, 0.4 mM) was added using a syringe. The reaction mixture was stirred for 48 h at room temperature. The reaction mixture was washed with brine and 10% aqueous solution of citric acid. The organic phase was collected and dried over magnesium sulfate. It was concentrated by rotary evaporation to give 230 mg crude product which was then purified by column chromatography on silica gel (50 g of silica, THF/hexane 4:6 with 5% iPrOH) to give 174 mg product. LCMS: 664 [M+1]⁺, 686 [M+Na]⁺.

### Example 8

### (4S,5S)—(N-Boc-valyloxy)methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate

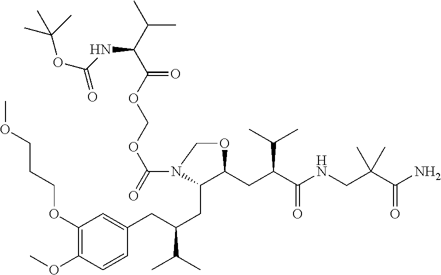 [SMILES: COCCCOc1cc(C[C@@H](C[C@H]2[C@H](C[C@H](C(=O)NCC(C)(C)C(N)=O)C(C)C)OCN2C(=O)OCOC(=O)[C@@H](NC(=O)OC(C)(C)C)C(C)C)C(C)C)ccc1OC]

The product was prepared using a method analogous to the method described in Example 4, Method A. LC-MS: 838 [M+1]⁺.

### Example 9

### (4S,5S)-valyloxymethyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate, trifluoroacetic acid salt

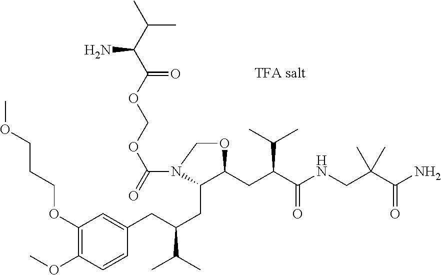 [SMILES: COCCCOc1cc(C[C@@H](C[C@H]2[C@H](C[C@H](C(=O)NCC(C)(C)C(N)=O)C(C)C)OCN2C(=O)OCOC(=O)[C@@H](N)C(C)C)C(C)C)ccc1OC]

(4S,5S)—(N-Boc-valyloxy)methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate (60 mg) was dissolved in dry DCM (1 ml) and 150-200 μl of TFA was added to the reaction mixture and stirred for 20 min. LC-MS shows full conversion of starting material peak to peak with mass M⁺737. Reaction mixture was concentrated by rotary evaporation and dried under vacuum to give 58 mg product as TFA salt. LC-MS: 737 [M+1]⁺.

### Example 10

### (4S,5S)—(N-CBz-valyloxy)methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate

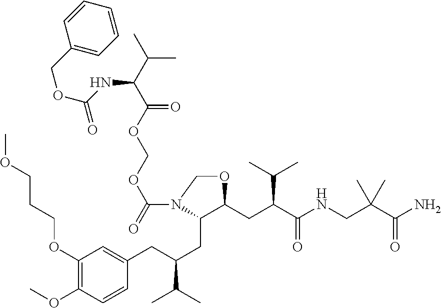 [SMILES: COCCCOc1cc(C[C@@H](C[C@H]2[C@H](C[C@H](C(=O)NCC(C)(C)C(N)=O)C(C)C)OCN2C(=O)OCOC(=O)[C@@H](NC(=O)OCc2ccccc2)C(C)C)C(C)C)ccc1OC]

The product was prepared using a method analogous to the method described in Example 4, LC-MS: mass peaks M⁺872 [M+1]⁺.

### Example 11

### (4S,5S)-(ethoxycarbonyloxy)methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate

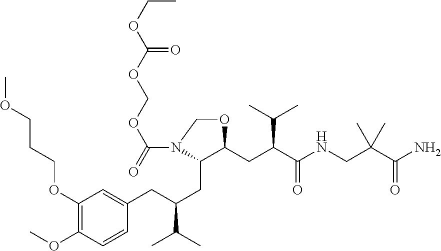 [SMILES: CCOC(=O)OCOC(=O)N1CO[C@@H](C[C@H](C(=O)NCC(C)(C)C(N)=O)C(C)C)[C@@H]1C[C@H](Cc1ccc(OC)c(OCCCOC)c1)C(C)C]

(4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine (100 mg, prepared using the method described in Example 1) and cesium carbonate (116 mg) were dissolved in 7 ml dry DMF. To the reaction mixture was added solid carbon dioxide and the reaction was kept under CO₂ atmosphere under an elevated pressure. The reaction mixture was stirred at room temperature for 50 min. Ethyl iodomethyl carbonate (61 mg) was added through a syringe to the stirred reaction mixture and reaction was kept under stirring overnight at room temperature. 40 ml water was then added and the reaction mixture was extracted with dichloromethane (3×20 ml). Combined organic phases were washed with brine and dried over MgSO₄. After evaporation in vacuo, the residue was purified by column chromatography on silica gel (EtOAc) to give 13 mg product. LC-MS: 710 [M+1]⁺.

### Example 12

### (4S,5S)-(isopropoxycarbonyloxy)methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate

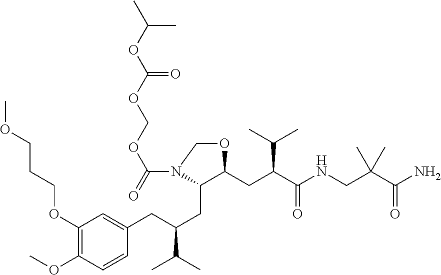 [SMILES: COCCCOc1cc(C[C@@H](C[C@H]2[C@H](C[C@H](C(=O)NCC(C)(C)C(N)=O)C(C)C)OCN2C(=O)OCOC(=O)OC(C)C)C(C)C)ccc1OC]

### Method A

The compounds was prepared using a method analogous to the method described in Example 11. LC-MS: 724 [M+1]⁺.

### Method B.

Sodium metal (15 mg) was dissolved in isopropanol and carbon dioxide was bubbled through the solution of sodium propylate obtained during 3 h. White precipitate formed. Reaction mixture was concentrated by rotary evaporation and dried under high vacuum. Sodium salt of isopropylcarbonate was dissolved in DMF (2 ml) and cesium iodide (400 mg) was added, which was followed by the addition of a DMF solution of chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (80 mg, preparation see Example 7). The reaction mixture was stirred overnight at room temperature. To the reaction mixture was added 10% citric acid aqueous solution (40 ml) and the mixture was extracted with dichloromethane (3×20 ml). The combined organic phases were washed with brine and dried over MgSO₄. After evaporation in vacuo, the residue was purified by column chromatography on silica (EtOAc) to give 23 mg product. LC-MS: 724 [M+1]⁺.

### Example 13

### Chloromethyl (4S,5S)-5-((2S)-2-{[(3-amino-2,2-dimethyl-3-oxopropyl)amino]carbonyl}-3-methylbutyl)-4-[(2S)-2-[4-methoxy-3-(3-methoxypropoxy)benzyl]-3-methylbutyl]-1,3-oxazolidine-3-carboxylate

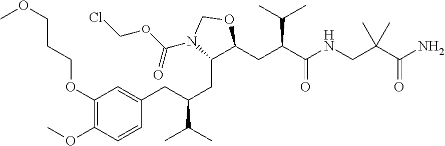 [SMILES: COCCCOc1cc(C[C@@H](C[C@H]2[C@H](C[C@H](C(=O)NCC(C)(C)C(N)=O)C(C)C)OCN2C(=O)OCCl)C(C)C)ccc1OC]

Aliskiren (1.1 g) was dissolved in 100 ml of THF and formaldehyde (37% solution in water, 150 ml) was added to the reaction mixture and stirred in ice bath overnight. LC-MC shows about 16% of starting material by TIC, then additional amount of formaline (10 ml) was added and stirred for additional 2 h, then a spoon of anhydrous magnesium sulfate was added and stirred for about 60 min. Reaction mixture was then filtered (LC-MS shows 100% conversion) and chloromethyl chloroformate (195 ml) and triethylamine (320 ml) were added to the stirred filtrate at cooling in an ice bath. LC-MS shows full conversion after 30 minutes stirring. Reaction mixture was concentrated by rotary evaporation, then mixed with 10% citric acid and brine and extracted into DCM. Organic extracts were washed with brine and dried over magnesium sulfate, concentrated and purified by column chromatography on silica gel (EtOAc) to give 961 mg of desired product as white foam. LCMS: 656.5 [M+1]⁺, 678.4 [M+Na]⁺; 654.4 [M−1]⁻

### Example 14

### Iodomethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

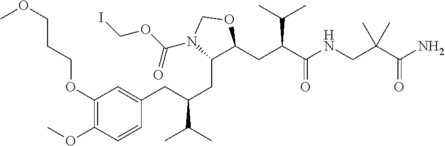 [SMILES: COCCCOc1cc(C[C@@H](C[C@H]2[C@H](C[C@H](C(=O)NCC(C)(C)C(N)=O)C(C)C)OCN2C(=O)OCI)C(C)C)ccc1OC]

Chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (191 mg) and sodium iodide (135 mg) were mixed with dry acetonitrile and heated under stirring at 75° C. for 1 h 20 min. Full conversion according to LC-MS. Reaction mixture was mixed with water and extracted into DCM, washed with brine, dried over MgSO₄. 186 mg of crude product was obtained, which was purified by column chromatography on silica gel (EtOAc) to give 72 mg of pure material as brown oil. Compound is not stable and decomposes rather rapidly. LCMS: 748.3 [M+1]⁺, 770.3 [M+Na]⁺. Compound is not stable and decomposes rather rapidly.

### Example 15

### {[(2S)-2-hydroxypropanoyl]oxy}methyl (4S,5S)-5-[(2S)-2-(3-amino-2,2-dimethyl-3-oxopropylaminocarbonyl)-3-methylbutyl]-4-{(2S)-2-[4-methoxy-3-(3-methoxypropoxy)benzyl]-3-methylbutyl}-1,3-oxazolidine-3-carboxylate

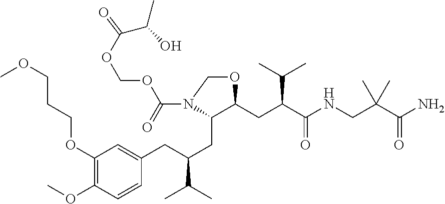 [SMILES: COCCCOc1cc(C[C@@H](C[C@H]2[C@H](C[C@H](C(=O)NCC(C)(C)C(N)=O)C(C)C)OCN2C(=O)OCOC(=O)[C@H](C)O)C(C)C)ccc1OC]

L-lactic acid lithium salt (18 mg) and cesium iodide (14 mg) was suspended in DMF (1 ml) and solution of chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (30 mg) in DMF (1 ml) was added and stirred at r.t. overnight. Reaction mixture was mixed with 10% citric acid (30 ml) and extracted into EtOAc (4×20 ml). Organic extracts were washed with brine and dried over MgSO₄. Concentrated by rotary evaporation and purified by column chromatography on silica gel (EtOAc/THF 9:1) to give 19 mg of colorless oil. LCMS: 710.5 [M+1]⁺, 732.5 [M+Na]⁺.

### Example 16

### {[(2S)-2-(ethoxymethoxy)propanoyl]oxy}methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate

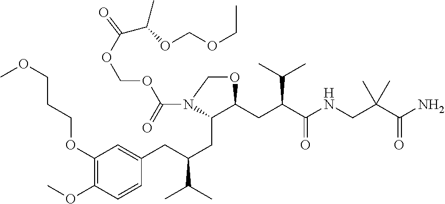 [SMILES: CCOCO[C@@H](C)C(=O)OCOC(=O)N1CO[C@@H](C[C@H](C(=O)NCC(C)(C)C(N)=O)C(C)C)[C@@H]1C[C@H](Cc1ccc(OC)c(OCCCOC)c1)C(C)C]

Cesium (2S)-2-(Ethoxymethoxy)propanoate (30 mg), cesium iodide (5 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (30 mg) was mixed with 1.5 ml of DMF and stirred at r.t. for 3 h. Reaction mixture was mixed with 40 ml of 10% citric acid and extracted into DCM (4×20 ml), washed with brine (3×20 ml) and dried over magnesium sulfate. Concentrated and purified by column chromatography on silica gel (EtOAc) and 18 mg of colorless oil was obtained. LCMS: 768.5 [M+1]⁺, 790.5 [M+Na]⁺.

### Example 17

### {(4S,5S)-5-[(2S)-2-(3-amino-2,2-dimethyl-3-oxopropylaminocarbonyl)-3-methylbutyl]-4-{(2S)-2-[4-methoxy-3-(3-methoxypropoxy)benzyl]-3-methylbutyl}-1,3-oxazolidin-3-yl-carbonyloxy}methyl morpholine-4-carboxylate

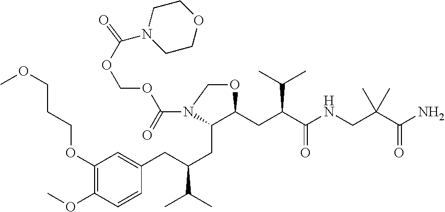 [SMILES: COCCCOc1cc(C[C@@H](C[C@H]2[C@H](C[C@H](C(=O)NCC(C)(C)C(N)=O)C(C)C)OCN2C(=O)OCOC(=O)N2CCOCC2)C(C)C)ccc1OC]

Morpholine (50 mg), cesium carbonate (80 mg), CsI (40 mg) were mixed with 2-3 ml of DMF in 2 neck 100 ml flask with balloon to keep CO₂ pressure and small amount of dry ice was added to the reaction mixture. Reaction mixture was stirred for about 2 h at r.t. Then chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (60 mg) in 2 ml of DMF was added to the reaction mixture and carbon dioxide tube was attached. Pressure of carbon dioxide was regulated from the cylinder. Reaction mixture was stirred at room temperature under CO₂ gas pressure for approximately 30 h. Progress was monitored by LC-MS. Reaction mixture was then mixed with 10% citric acid solution (30 ml) and some brine and extracted into DCM. Combined organic extract was washed with brine (20 ml) and dried over MgSO₄ and kept under high vacuum to remove residual DMF. Purified by column chromatography on YMC silica gel (EtOAc/THF 9:1) and 32 mg of pure (LC-MS) product was obtained as colorless oil. LCMS: 751.5 [M+1]⁺, 773.5 [M+Na]⁺; 749.5 [M−1]⁻

### Example 18

### (4S,5S) nicotinoyloxymethyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate

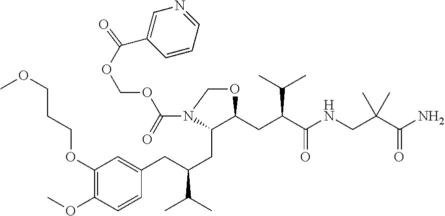 [SMILES: COCCCOc1cc(C[C@@H](C[C@H]2[C@H](C[C@H](C(=O)NCC(C)(C)C(N)=O)C(C)C)OCN2C(=O)OCOC(=O)c2cccnc2)C(C)C)ccc1OC]

Nicotinic acid (25 mg), cesium carbonate (as a base, 67 mg), cesium iodide (17 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (27 mg) were mixed with dry DMF and stirred for about 27 h. Reaction progress was monitored by LC-MS. Reaction mixture was mixed with water (acidified to neutral pH with 10% citric acid) and extracted into DCM (4×20 ml). Combined organic phases were washed with brine, dried over anhydrous MgSO₄ and concentrated by rotary evaporation and kept under high vacuum to remove traces of DMF left. Purified by column chromatography on YMC silica gel (EtOAc/absTHF 9:1) and 25 mg of desired product was obtained. LCMS: 743.5 [M+1]⁺, 765.4 [M+Na]⁺; 741.4 [M−1]⁻

### Example 19

### (4S,5S) [(pyridine-2-yl)carbonyloxy]methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate

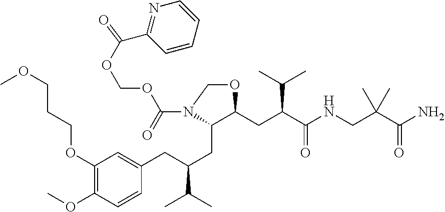 [SMILES: COCCCOc1cc(C[C@@H](C[C@H]2[C@H](C[C@H](C(=O)NCC(C)(C)C(N)=O)C(C)C)OCN2C(=O)OCOC(=O)c2ccccn2)C(C)C)ccc1OC]

The compound was prepared using a method analogous to the method described in Example 18 from 2-picolinic acid (20 mg), cesium iodide (20 mg), cesium carbonate (as a base, 62 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (27 mg) to give 19 mg of desired product. LCMS: 743.5 [M+1]⁺, 765.4 [M+Na]⁺; 741.5 [M−1]⁻

### Example 20

### [(2-methylpropoxycarbonyl)oxy]methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate

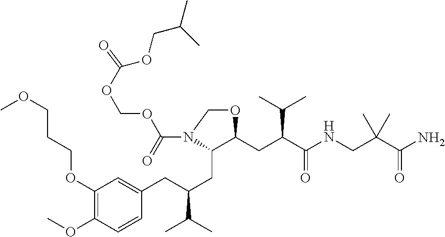 [SMILES: COCCCOc1cc(C[C@@H](C[C@H]2[C@H](C[C@H](C(=O)NCC(C)(C)C(N)=O)C(C)C)OCN2C(=O)OCOC(=O)OCC(C)C)C(C)C)ccc1OC]

The compound was prepared using a method analogous to the method described in literature (Kim S-I, Chu F, Dueno E, Jung K W, J. Org. Chem., 64 (1999), 4578) from cesium carbonate (126 mg), tetrabutylammonium iodide (13 mg) and isobutanol (0.1 ml) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (20 mg) to give 21 mg of desired product. LCMS: 738.5 [M+1]⁺, 760.4 [M+Na]⁺; 736.5 [M−1]⁻

### Example 21

### {[2-methyl-2-(ethoxymethoxy)propanoyl]oxy}methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate

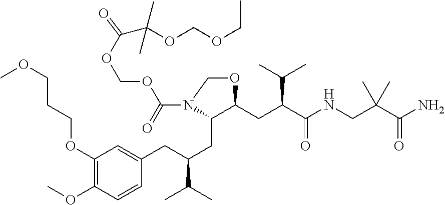 [SMILES: CCOCOC(C)(C)C(=O)OCOC(=O)N1CO[C@@H](C[C@H](C(=O)NCC(C)(C)C(N)=O)C(C)C)[C@@H]1C[C@H](Cc1ccc(OC)c(OCCCOC)c1)C(C)C]

The compound was prepared using a method analogous to the method described in Example 16 from cesium 2-(ethoxymethoxy)-2-methylpropanoate (15 mg), cesium iodide (10 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (24 mg) to give 17 mg of desired product as an oil. LCMS: 782.5 [M+1]⁺, 804.5 [M+Na]⁺; 780.6 [M−1]⁻

### Example 22

### {[(pyridin-3-ylmethoxy)carbonyl]oxy}methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate

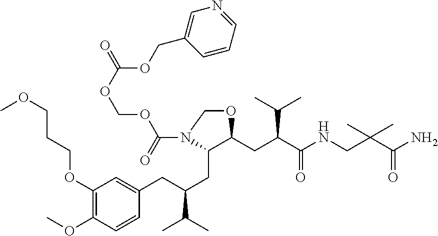 [SMILES: COCCCOc1cc(C[C@@H](C[C@H]2[C@H](C[C@H](C(=O)NCC(C)(C)C(N)=O)C(C)C)OCN2C(=O)OCOC(=O)OCc2cccnc2)C(C)C)ccc1OC]

The compound was prepared using a method analogous to the method described in Example 20 from 3-pyridinemethanol (100 mg), cesium carbonate (180 mg), TBAI (19 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (30 mg). Purification by column chromatography on YMC silica gel (EtOAc, 7% of MeOH) gave 15 mg of desired product as an oil. LCMS: 773.4 [M+1]⁺, 795.5 [M+Na]⁺; 771.5 [M−1]⁻

### Example 23

### [(2-methyl-3-morpholin-4-ylpropanoyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

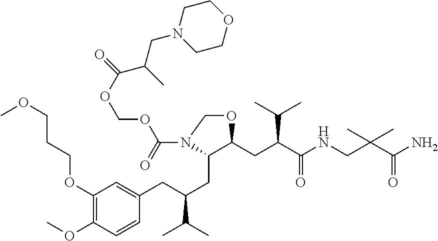 [SMILES: COCCCOc1cc(C[C@@H](C[C@H]2[C@H](C[C@H](C(=O)NCC(C)(C)C(N)=O)C(C)C)OCN2C(=O)OCOC(=O)C(C)CN2CCOCC2)C(C)C)ccc1OC]

The compound was prepared using a method analogous to the method described in Example 16 from cesium 2-methyl-3-(morpholin-4-yl)propanoate (30 mg), cesium iodide (24 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (30 mg) to give 33 mg of desired product as an oil. LCMS: 793.5 [M+1]⁺, 815.5 [M+Na]⁺

### Example 24

### (1-methylpiperidine-4-carbonyloxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

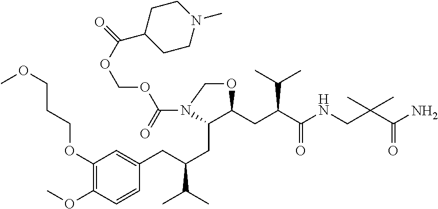 [SMILES: COCCCOc1cc(C[C@@H](C[C@H]2[C@H](C[C@H](C(=O)NCC(C)(C)C(N)=O)C(C)C)OCN2C(=O)OCOC(=O)C2CCN(C)CC2)C(C)C)ccc1OC]

The compound was prepared using a method analogous to the method described in Example 18 from 1-methylpiperidine-4-carboxylic acid (19 mg), cesium iodide (23 mg), cesium carbonate (as a base, 60 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) to give 40 mg of desired product as foam. LCMS: 763.5 [M+1]⁺, 785.4 [M+Na]⁺; 761.6 [M−1]⁻

### Example 25

### {[(1,3-dioxan-5-yl-oxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

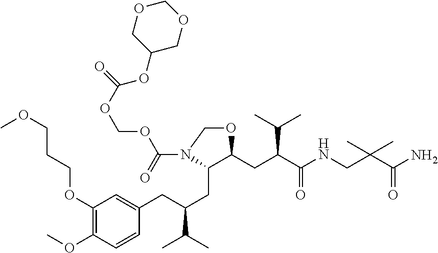 [SMILES: COCCCOc1cc(C[C@@H](C[C@H]2[C@H](C[C@H](C(=O)NCC(C)(C)C(N)=O)C(C)C)OCN2C(=O)OCOC(=O)OC2COCOC2)C(C)C)ccc1OC]

The compound was prepared using a method analogous to the method described in Example 20 from 5-hydroxy-1,3-dioxane (40 mg), cesium carbonate (100 mg), tetrabutylammonium iodide (28 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg). Purification by column chromatography on YMC silica gel (EtOAc) gave 37 mg of desired product as an oil. LCMS: 768.5 [M+1]⁺, 790.4 [M+Na]⁺; 766.5 [M−1]⁻

### Example 26

### {[(1,3-dioxolan-4-ylmethoxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (racemate)

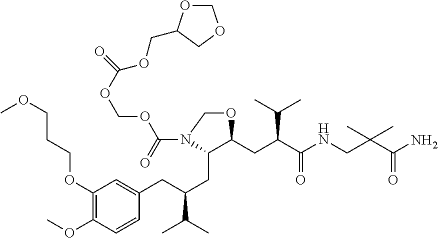 [SMILES: COCCCOc1cc(C[C@@H](C[C@H]2[C@H](C[C@H](C(=O)NCC(C)(C)C(N)=O)C(C)C)OCN2C(=O)OCOC(=O)OCC2COCO2)C(C)C)ccc1OC]

The compound was prepared using a method analogous to the method described in Example 20 from (1,3-dioxolan-4-yl)methanol (50 mg, racemate), cesium carbonate (100 mg), tetrabutylammonium iodide (28 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg). Purification by column chromatography on YMC silica gel (EtOAc) gave 50 mg of desired product as an oil. LCMS: 768.5 [M+1]⁺, 790.5 [M+Na]⁺; 766.5 [M−1]⁻

### Example 27

### [(3-hydroxy-2,2-dimethylpropanoyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

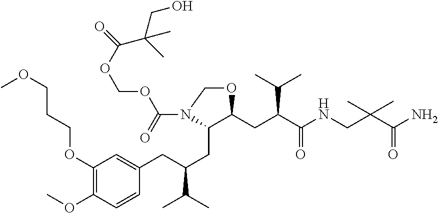 [SMILES: COCCCOc1cc(C[C@@H](C[C@H]2[C@H](C[C@H](C(=O)NCC(C)(C)C(N)=O)C(C)C)OCN2C(=O)OCOC(=O)C(C)(C)CO)C(C)C)ccc1OC]

The compound was prepared using a method analogous to the method described in Example 16 from cesium 2,2-dimethyl-3-hydroxypropanoate (77 mg), cesium iodide (23 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) to give 20 mg of desired product as an oil. LCMS: 738.5 [M+1]⁺, 760.5 [M+Na]⁺

### Example 28

### {[(4-methoxybenzyloxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

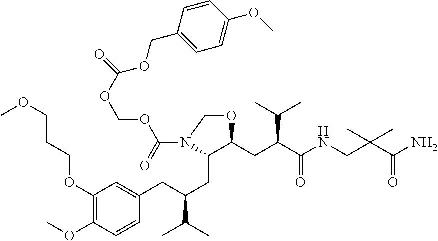 [SMILES: COCCCOc1cc(C[C@@H](C[C@H]2[C@H](C[C@H](C(=O)NCC(C)(C)C(N)=O)C(C)C)OCN2C(=O)OCOC(=O)OCc2ccc(OC)cc2)C(C)C)ccc1OC]

The compound was prepared using a method analogous to the method described in Example 20 from 4-methoxybenzyl alcohol (30 mg), cesium carbonate (60 mg), TBAI (17 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (30 mg) to give 19 mg of desired product as white foam. LCMS: 802.511 [M+1]⁺, 824.5 [M+Na]⁺; 800.5 [M−1]⁻

### Example 29

### {[benzyloxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

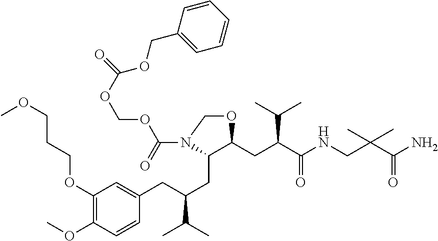 [SMILES: COCCCOc1cc(C[C@@H](C[C@H]2[C@H](C[C@H](C(=O)NCC(C)(C)C(N)=O)C(C)C)OCN2C(=O)OCOC(=O)OCc2ccccc2)C(C)C)ccc1OC]

The compound was prepared using a method analogous to the method described in Example 20 from benzyl alcohol (15 mg), cesium carbonate (67 mg), tetrabutylammonium iodide (17 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (33 mg) to give 19 mg of desired product as white foam. LCMS: 772.5 [M+1]⁺, 794.5 [M+Na]⁺; 770.6 [M−1]⁻

### Example 30

### [(pyridine-4-yl)carbonyloxy]methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate

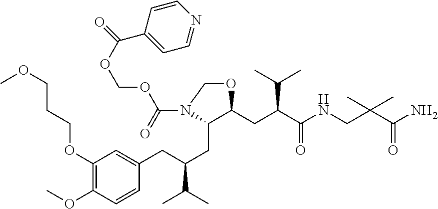 [SMILES: COCCCOc1cc(C[C@@H](C[C@H]2[C@H](C[C@H](C(=O)NCC(C)(C)C(N)=O)C(C)C)OCN2C(=O)OCOC(=O)c2ccncc2)C(C)C)ccc1OC]

The compound was prepared using a method analogous to the method described in Example 18 from isonicotinic acid (6 mg), cesium iodide (14 mg), cesium carbonate (as a base, 23 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (31 mg) to give 16 mg of desired product. LCMS: 743.5 [M+1]⁺, 765.4 [M+Na]⁺; 741.4 [M−1]⁻

### Example 31

### {[(1-methyl-1H-imidazol-4-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

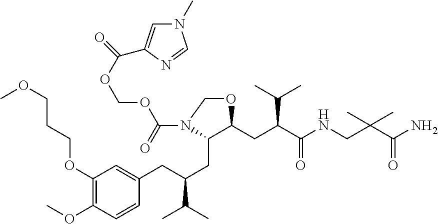 [SMILES: COCCCOc1cc(C[C@@H](C[C@H]2[C@H](C[C@H](C(=O)NCC(C)(C)C(N)=O)C(C)C)OCN2C(=O)OCOC(=O)c2cn(C)cn2)C(C)C)ccc1OC]

The compound was prepared using a method analogous to the method described in Example 18 from 1-methylimidazole-4-carboxylic acid (8 mg), cesium iodide (19 mg), cesium carbonate (as a base, 21 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (33 mg). Purification by column chromatography on YMC silica gel (EtOAc with 10% MeOH) gave 19 mg of desired product as an oil to give 16 mg of desired product. LCMS: 746.5 [M+1]⁺, 768.5 [M+Na]⁺; 744.5 [M−1]⁻¹

### Example 32

### [(1,3-dioxan-5-ylcarbonyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

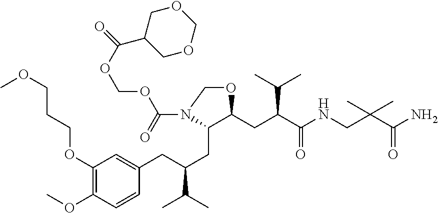 [SMILES: COCCCOc1cc(C[C@@H](C[C@H]2[C@H](C[C@H](C(=O)NCC(C)(C)C(N)=O)C(C)C)OCN2C(=O)OCOC(=O)C2COCOC2)C(C)C)ccc1OC]

The compound was prepared using a method analogous to the method described in Example 18 from 1,3-dioxane-5-carboxylic acid (11 mg, obtained using procedures described in Finlay MacCorquodale et al, J. Chem. Soc., Perkin Trans 2, 1991, 1893-9), cesium iodide (19 mg), cesium carbonate (as a base, 29 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) to give 30 mg of desired product as white foam. LCMS: 752.5 [M+1]⁺, 774.5 [M+Na]⁺

### Example 33

### {[(1-methyl-1H-imidazol-2-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

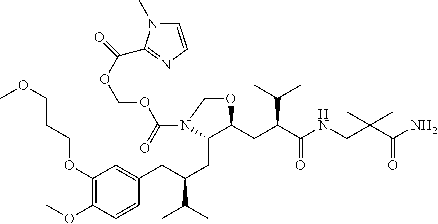 [SMILES: COCCCOc1cc(C[C@@H](C[C@H]2[C@H](C[C@H](C(=O)NCC(C)(C)C(N)=O)C(C)C)OCN2C(=O)OCOC(=O)c2nccn2C)C(C)C)ccc1OC]

The compound was prepared using a method analogous to the method described in Example 31 from 1-methyl-1H-imidazole-2-carboxylic acid (12 mg), cesium iodide (27 mg), cesium carbonate (as a base, 45 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) to give 16 mg of desired product as colorless oil. LCMS: 746.5 [M+1]⁺, 768.5 [M+Na]⁺; 744.5 [M−1]⁻

### Example 34

### ({[(1-methyl-1H-imidazol-4-yl)methoxy]carbonyl}oxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

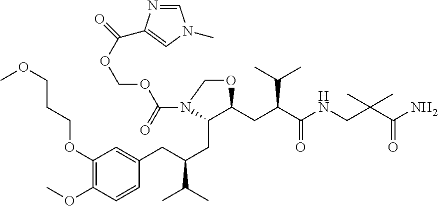 [SMILES: COCCCOc1cc(C[C@@H](C[C@H]2[C@H](C[C@H](C(=O)NCC(C)(C)C(N)=O)C(C)C)OCN2C(=O)OCOC(=O)c2cn(C)cn2)C(C)C)ccc1OC]

The compound was prepared using a method analogous to the method described in Example 20 from 1-methylimidazole-4-methanol (18 mg), cesium carbonate (108 mg), tetrabutylammonium iodide (25 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg). Purification by column chromatography on YMC silica gel (EtOAc with 10% MeOH) gave 26 mg of desired product as white foam. LCMS: 776.5 [M+1]⁺, 798.5 [M+Na]⁺; 774.5 [M−1]⁻

### Example 35

### ({[(1-methylpiperidin-4-yl)oxy]carbonyl}oxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

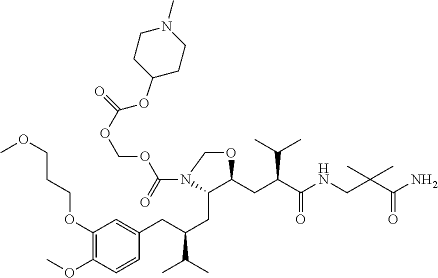 [SMILES: COCCCOc1cc(C[C@@H](C[C@H]2[C@H](C[C@H](C(=O)NCC(C)(C)C(N)=O)C(C)C)OCN2C(=O)OCOC(=O)OC2CCN(C)CC2)C(C)C)ccc1OC]

1-Methyl-4-piperidinol (46 mg), cesium carbonate (53 mg), cesium iodide (25 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (100 mg) were mixed with dry DMF and bubbled with carbon dioxide at stirring. Reaction progress was monitored by LC-MS. Full conversion was achieved after 40 h. Reaction mixture was acidified to pH 6 with 10% citric acid solution (20 ml) and some water and brine was added and extracted into DCM. Combined organic extract was washed with brine (20 ml), dried over MgSO₄, filtered and the crude oil was kept in high vacuum to remove residual DMF to obtain 126 mg of crude product as slightly yellow oil. Purification by preparative HPLC (C18 column; acetonitrile/water with 0.1% TFA) gave 32 mg of desired product as an oil (in form of TFA salt). LCMS: 779.5 [M+1]⁺, 801.5 [M+Na]⁺; 777.5 [M−1]⁻

### Example 36

### [(1-methylpiperidin-4-yl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

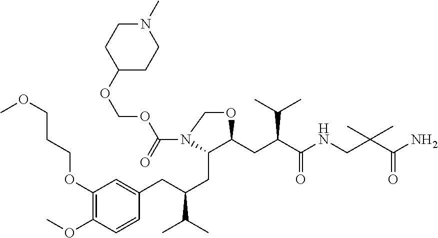 [SMILES: COCCCOc1cc(C[C@@H](C[C@H]2[C@H](C[C@H](C(=O)NCC(C)(C)C(N)=O)C(C)C)OCN2C(=O)OCOC2CCN(C)CC2)C(C)C)ccc1OC]

Compound was obtained in the reaction described in Example 35. Product was isolated by preparative HPLC from crude mixture (Example 35) as 39 mg of white foam (TFA salt). LCMS: 735.5 [M+1]⁺; 733.5 [M−1]⁻

### Example 37

### ({[(1-methylpiperidin-4-yl)methoxy]carbonyl}oxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

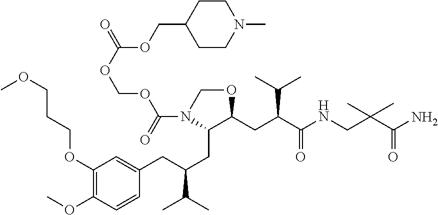 [SMILES: COCCCOc1cc(C[C@@H](C[C@H]2[C@H](C[C@H](C(=O)NCC(C)(C)C(N)=O)C(C)C)OCN2C(=O)OCOC(=O)OCC2CCN(C)CC2)C(C)C)ccc1OC]

The compound was prepared using a method analogous to the method described in Example 20 from 1-methyl-4-piperidinemethanol (20 mg), cesium carbonate (112 mg), TBAI (22 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg). Purification by column chromatography on YMC silica gel (EtOAc with 10% MeOH) gave 10.4 mg of desired product as colorless oil. LCMS: 793.5 [M+1]⁺, 815.5 [M+Na]⁺; 791.6 [M−1]⁻

### Example 38

### {[(1,3-dioxan-5-ylmethoxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

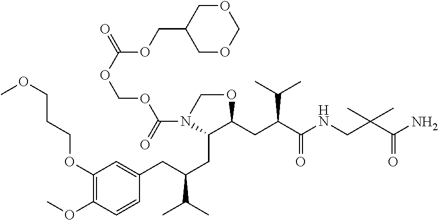 [SMILES: COCCCOc1cc(C[C@@H](C[C@H]2[C@H](C[C@H](C(=O)NCC(C)(C)C(N)=O)C(C)C)OCN2C(=O)OCOC(=O)OCC2COCOC2)C(C)C)ccc1OC]

Cesium carbonate (230 mg), cesium iodide (18 mg) and 1,3-dioxane-5-methanol (20 mg, obtained using procedures described in Finlay MacCorquodale et al, J. Chem. Soc., Perkin Trans 2, (1991) 1893-9) were mixed with 1-2 ml of DMF and bubbled at stirring with carbon dioxide for about 1 h at r.t. Then chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) in 2 ml of DMF was added to the reaction mixture and carbon dioxide was bubbled at stirring overnight. Reaction progress was monitored by LC-MS. After about 42 h reaction mixture was mixed with 10% citric acid solution (20 ml) and some brine and extracted into DCM. Combined organic extract was washed with brine (20 ml) and dried over MgSO₄, crude oil was dried in high vacuum to remove residual DMF. Purified by column chromatography on YMC silica gel (EtOAc) gave 19 mg of white foam. LCMS: 782.5 [M+1]⁺, 804.5 [M+Na]⁺; 780.5 [M−1]⁻

### Example 39

### (pyridin-3-yloxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

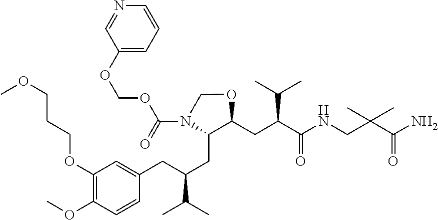 [SMILES: COCCCOc1cc(C[C@@H](C[C@H]2[C@H](C[C@H](C(=O)NCC(C)(C)C(N)=O)C(C)C)OCN2C(=O)OCOc2cccnc2)C(C)C)ccc1OC]

Chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) was mixed with cesium carbonate (110 mg), cesium iodide (21 mg), and 3-hydroxypyridine (32 mg). Then 2 ml DMF was added and the reaction mixture was stirred for about 2-3 h. Reaction mixture was then mixed with 10% citric acid solution (20 ml) and some brine and extracted into DCM. Combined organic extract was washed with brine (20 ml) and dried over MgSO₄. Crude oil was kept under high vacuum to remove residual DMF. Purified by column chromatography on YMC silica gel (EtOAc:MeOH 9:1) gave 50 mg of slightly yellow oil. LCMS: 715.5 [M+1]⁺, 737.5 [M+Na]⁺; 713.5 [M−1]⁻

### Example 40

### {[(3-methoxy-2,2-dimethyl-3-oxopropoxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

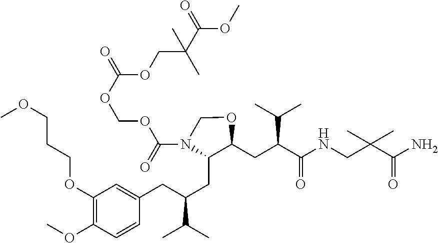 [SMILES: COCCCOc1cc(C[C@@H](C[C@H]2[C@H](C[C@H](C(=O)NCC(C)(C)C(N)=O)C(C)C)OCN2C(=O)OCOC(=O)OCC(C)(C)C(=O)OC)C(C)C)ccc1OC]

The compound was prepared using a method analogous to the method described in Example 38 from methyl 2,2-dimethyl-3-hydroxypropionate (20 mg), cesium carbonate (80 mg), cesium iodide (30 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (53 mg) to give 47 mg of desired product as colorless oil. LCMS: 796.5 [M+1]⁺, 818.5 [M+Na]⁺; 794.6 [M−1]⁻

### Example 41

### {[(dimethylamino)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

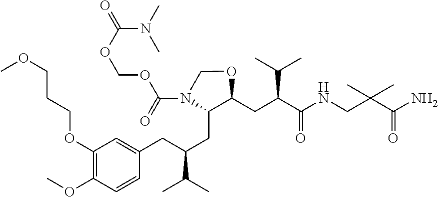 [SMILES: COCCCOc1cc(C[C@@H](C[C@H]2[C@H](C[C@H](C(=O)NCC(C)(C)C(N)=O)C(C)C)OCN2C(=O)OCOC(=O)N(C)C)C(C)C)ccc1OC]

The compound was prepared using a method analogous to the method described in Example 17 (bubbling with carbon dioxide was used instead of addition of dry ice to the reaction mixture) from dimethyl hydrochloride (36 mg), cesium carbonate (277 mg), cesium iodide (36 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (60 mg) to give 30 mg of desired product as colorless oil. LCMS: 709.5 [M+1]⁺, 731.5 [M+Na]⁺; 707.5 [M−1]⁻

### Example 42

### [({1-[(tert-butoxycarbonyl)amino]cyclopropyl}carbonyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

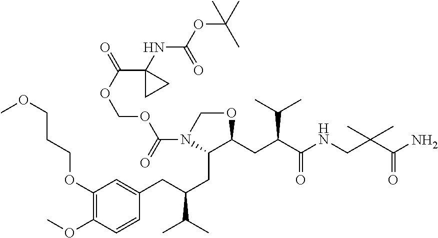 [SMILES: COCCCOc1cc(C[C@@H](C[C@H]2[C@H](C[C@H](C(=O)NCC(C)(C)C(N)=O)C(C)C)OCN2C(=O)OCOC(=O)C2(NC(=O)OC(C)(C)C)CC2)C(C)C)ccc1OC]

The compound was prepared using a method analogous to the method described in Example 18 from 1-tert-butoxycarbonylaminocyclopropylcarboxylic acid (20 mg), cesium iodide (19 mg), cesium carbonate (30 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) to give 50 mg of desired product as an oil. LCMS: 821.5 [M+1]⁺, 843.5 [M+Na]⁺; 819.7 [M−1]⁻

### Example 43

### {[(1-aminocyclopropyl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate, trifluoroacetate

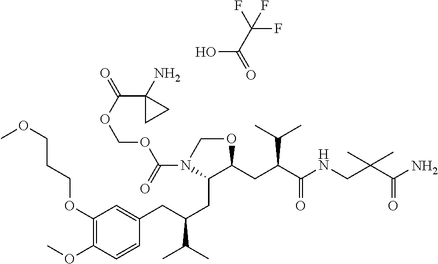 [SMILES: COCCCOc1cc(C[C@@H](C[C@H]2[C@H](C[C@H](C(=O)NCC(C)(C)C(N)=O)C(C)C)OCN2C(=O)OCOC(=O)C2(N)CC2)C(C)C)ccc1OC ; O=C(O)C(F)(F)F]

The compound was prepared using a method analogous to the method described in Example 9 from [({1-[(tert-butoxycarbonyl)amino]cyclopropyl}carbonyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (52 mg) to give 52 mg of desired product (as TFA salt) as an oil. LCMS: 721.5 [M+1]⁺, 743.5 [M+Na]⁺; 719.5 [M−1]⁻

### Example 44

### {[(1-methyl-1H-imidazol-5-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

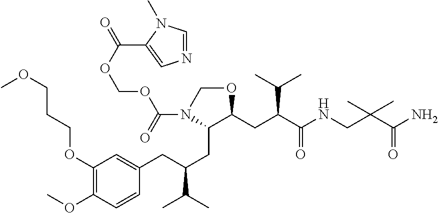 [SMILES: COCCCOc1cc(C[C@@H](C[C@H]2[C@H](C[C@H](C(=O)NCC(C)(C)C(N)=O)C(C)C)OCN2C(=O)OCOC(=O)c2cncn2C)C(C)C)ccc1OC]

The compound was prepared using a method analogous to the method described in Example 31 from 3-methyl-3H-imidazole-4-carboxylic acid (23 mg), cesium iodide (57 mg), cesium carbonate (90 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (109 mg) to give 65 mg of desired product as colorless oil. LCMS: 746.5 [M+1]⁺, 768.5 [M+Na]⁺; 744.5 [M−1]⁻

### Example 45

### 1-Chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-oxazolidine-3-carboxylate

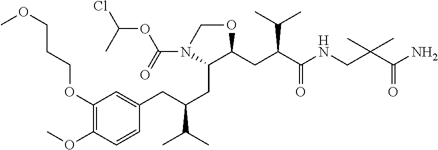 [SMILES: COCCCOc1cc(C[C@@H](C[C@H]2[C@H](C[C@H](C(=O)NCC(C)(C)C(N)=O)C(C)C)OCN2C(=O)OC(C)Cl)C(C)C)ccc1OC]

The compound was prepared using a method analogous to the method described in Example 13 from Aliskiren (550 mg) and 1-chloroethyl chloroformate (131 ml) to give 335 mg of desired product as white foam. LCMS: 670.5 [M+1]⁺, 792.5 [M+Na]⁺

### Example 46

### 1-{[(1-methyl-1H-imidazol-2-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

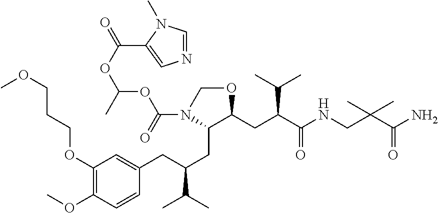 [SMILES: COCCCOc1cc(C[C@@H](C[C@H]2[C@H](C[C@H](C(=O)NCC(C)(C)C(N)=O)C(C)C)OCN2C(=O)OC(C)OC(=O)c2cncn2C)C(C)C)ccc1OC]

The compound was prepared using a method analogous to the method described in Example 31 from 1-methyl-1H-imidazole-2-carboxylic acid (8 mg), cesium carbonate (30 mg), cesium iodide (16 mg) and 1-chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-oxazolidine-3-carboxylate (40 mg) to give 8 mg of desired product as colorless oil. LCMS: 760.5 [M+1]⁺, 782.5 [M+Na]⁺; 758.5 [M−]⁻

### Example 47

### 1-{1-[(tert-butoxycarbonyl)amino]cyclopropanecarbonyloxy}-ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

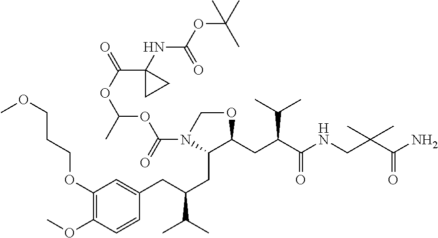 [SMILES: COCCCOc1cc(C[C@@H](C[C@H]2[C@H](C[C@H](C(=O)NCC(C)(C)C(N)=O)C(C)C)OCN2C(=O)OC(C)OC(=O)C2(NC(=O)OC(C)(C)C)CC2)C(C)C)ccc1OC]

The compound was prepared using a method analogous to the method described in Example 42 from 1-tert-butoxycarbonylaminocyclopropylcarboxylic acid (21 mg), cesium iodide (23 mg), cesium carbonate (27 mg), and 1-chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-oxazolidine-3-carboxylate (50 mg) to give 50 mg of desired product as an oil. LCMS: 835.5 [M+1]⁺, 857.5 [M+Na]⁺; 833.5 [M−1]⁻

### Example 48

### 1-(1-aminocyclopropanecarbonyloxy)ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate, trifloroacetate

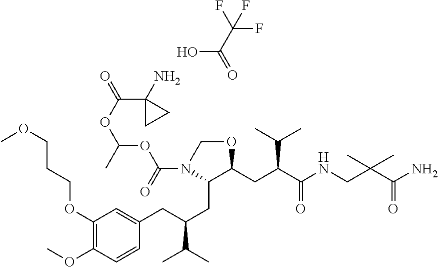 [SMILES: O=C(O)C(F)(F)F ; COCCCOc1cc(C[C@@H](C[C@H]2[C@H](C[C@H](C(=O)NCC(C)(C)C(N)=O)C(C)C)OCN2C(=O)OC(C)OC(=O)C2(N)CC2)C(C)C)ccc1OC]

The compound was prepared using a method analogous to the method described in Example 43 from 1-{1-[(tert-butoxycarbonyl)amino]cyclopropanecarbonyloxy}-ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (46 mg) to give 55 mg of desired product (as TFA salt) as an oil. LCMS: 735.5 [M+1]⁺, 757.5 [M+Na]⁺; 733.5 [M−1]⁻

### Example 49

### {[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methyl tert-butyl (2E)-but-2-enedioate

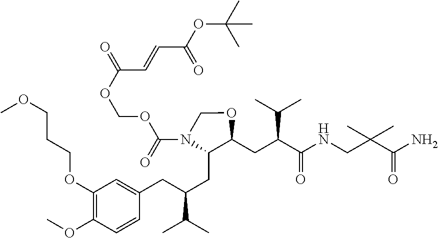 [SMILES: COCCCOc1cc(C[C@@H](C[C@H]2[C@H](C[C@H](C(=O)NCC(C)(C)C(N)=O)C(C)C)OCN2C(=O)OCOC(=O)/C=C/C(=O)OC(C)(C)C)C(C)C)ccc1OC]

The compound was prepared using a method analogous to the method described in Example 18 from fumaric acid mono tert-butylate (20 mg), cesium iodide (20 mg), cesium carbonate (as a base, 38 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) to give 44 mg of desired product as colorless oil. LCMS: 792.5 [M+1]⁺, 814.5 [M+Na]⁺

### Example 50

### 1-({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methoxy)oxo-(2E)-but-2-enoic acid

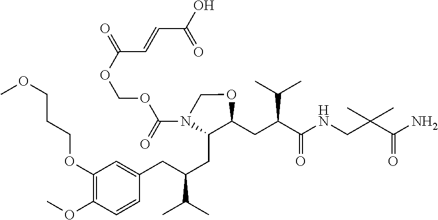 [SMILES: COCCCOc1cc(C[C@@H](C[C@H]2[C@H](C[C@H](C(=O)NCC(C)(C)C(N)=O)C(C)C)OCN2C(=O)OCOC(=O)/C=C/C(=O)O)C(C)C)ccc1OC]

{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methyl tert-butyl (2E)-but-2-enedioate (44 mg) was dissolved in dry DCM (1 ml) and trifluoroactic acid (0.5 ml) was added to the stirred solution at r.t. Reaction course was monitored by LC-MS. After 1 h stirring reaction mixture was concentrated by rotary evaporation and coevaporated with water (2×1 ml), methanol (2 ml) and 3 times with toluene to remove excess of trifluoromethyl acid and water. Product obtained was dried under high vacuum for 18-20 h to give desired material as slightly rose oil 29 mg. LCMS: 736.4 [M+1]⁺, 758.4 [M+Na]⁺; 734.4 [M−1]⁻

### Example 51

### {[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methyl 1-azabicyclo[2.2.1]heptane-4-carboxylate

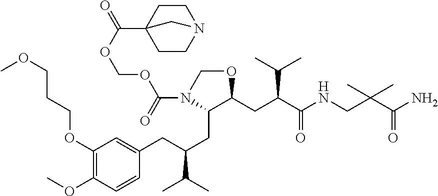 [SMILES: COCCCOc1cc(C[C@@H](C[C@H]2[C@H](C[C@H](C(=O)NCC(C)(C)C(N)=O)C(C)C)OCN2C(=O)OCOC(=O)C23CCN(CC2)C3)C(C)C)ccc1OC]

The compound was prepared using a method analogous to the method described in Example 16 from 1-azabicyclo[2.2.1]heptane-4-carboxylic acid cesium salt (76 mg, obtained by hydrolysis of corresponding ethyl ester hydrobromide in the presence of excess of cesium carbonate; ethyl 1-azabicyclo[2.2.1]heptane-4-carboxylate was prepared by literature procedure described in Eckhardt W et al, Helv. Chim. Acta, 55(7), 1972, 2432), cesium iodide (34 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) to give 40 mg of desired product as yellow oil without chromatographic purification. Purity of the product obtained was 80% (by UV at 230 nm). LCMS: 761.5 [M+1]⁺, 783.5 [M+Na]⁺; 759.5 [M−1]⁻

### Example 52

### {([(1-{[(tert-butoxycarbonyl)amino]methyl}cyclopropyl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

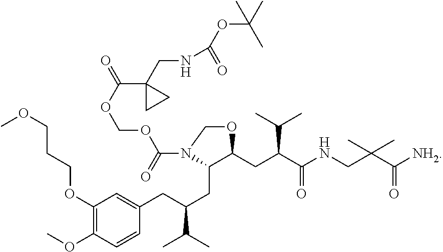 [SMILES: COCCCOc1cc(C[C@@H](C[C@H]2[C@H](C[C@H](C(=O)NCC(C)(C)C(N)=O)C(C)C)OCN2C(=O)OCOC(=O)C2(CNC(=O)OC(C)(C)C)CC2)C(C)C)ccc1OC]

The compound was prepared using a method analogous to the method described in Example 42 from 1-tert-butoxycarbonylaminomethylcyclopropylcarboxylic acid (20 mg), cesium iodide (20 mg), cesium carbonate (as a base, 37 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) to give 45 mg of desired product as colorless oil. LCMS: 835.5 [M+1]⁺, 857.5 [M+Na]⁺

### Example 53

{1-[({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methoxy)carbonyl]cyclopropyl}methanaminium trifluoroacetate

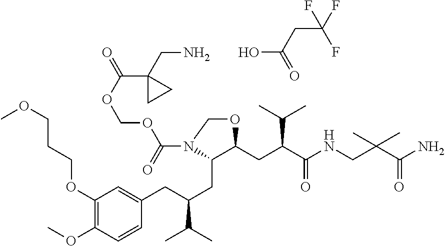

The compound was prepared using a method analogous to the method described in Example 43 from {[(1-{[(tert-butoxycarbonyl)amino]methyl}cyclopropyl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (42 mg) to give 39 mg of desired product (as TFA salt) as rose-brown foam.

LCMS: 735.5 [M+1]⁺, 757.5 [M+Na]⁺; 733.6 [M−1]⁻

### Example 54

### 1-{[(1-methyl-1H-imidazol-4-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

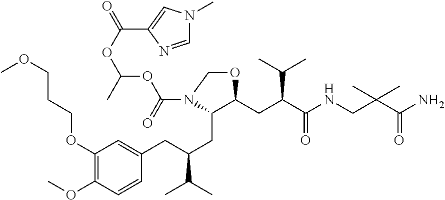 [SMILES: COCCCOc1cc(C[C@@H](C[C@H]2[C@H](C[C@H](C(=O)NCC(C)(C)C(N)=O)C(C)C)OCN2C(=O)OC(C)OC(=O)c2cn(C)cn2)C(C)C)ccc1OC]

The compound was prepared using a method analogous to the method described in Example 31 from 1-methylimidazole-4-carboxylic acid (36 mg), cesium iodide (59 mg), cesium carbonate (75 mg), and 1-chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-oxazolidine-3-carboxylate (85 mg). Product was purified by preparative HPLC to give 18.9 mg of desired product as foam. LCMS: 760.5 [M+1]⁺, 782.4 [M+Na]⁺; 758.5 [M−1]⁻

### Example 55

### 1-{[(pyridin-3-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

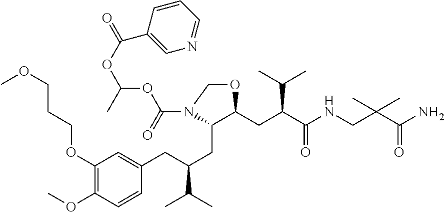 [SMILES: COCCCOc1cc(C[C@@H](C[C@H]2[C@H](C[C@H](C(=O)NCC(C)(C)C(N)=O)C(C)C)OCN2C(=O)OC(C)OC(=O)c2cccnc2)C(C)C)ccc1OC]

The compound was prepared using a method analogous to the method described in Example 18 from nicotinic acid (19 mg), cesium iodide (21 mg), cesium carbonate (57 mg), and 1-chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-oxazolidine-3-carboxylate (50 mg) to give 38 mg of desired product as colorless oil. LCMS: 757.5 [M+1]⁺, 779.5 [M+Na]⁺; 755.5 [M−1]⁻

### Example 56

### 1-{[(pyridin-2-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

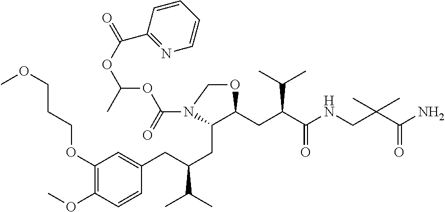 [SMILES: COCCCOc1cc(C[C@@H](C[C@H]2[C@H](C[C@H](C(=O)NCC(C)(C)C(N)=O)C(C)C)OCN2C(=O)OC(C)OC(=O)c2ccccn2)C(C)C)ccc1OC]

The compound was prepared using a method analogous to the method described in Example 18 from picolinic acid (20 mg), cesium iodide (22 mg), cesium carbonate (as a base, 53 mg), and 1-chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-oxazolidine-3-carboxylate (53 mg) to give 26 mg of desired product as colorless oil. LCMS: 757.4 [M+1]⁺, 779.4 [M+Na]⁺; 755.5 [M−1]⁻

### Example 57

### 1-{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}ethyl tert-butyl butanedioate

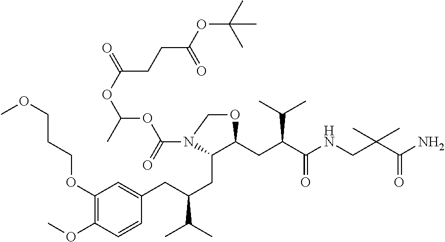 [SMILES: COCCCOc1cc(C[C@@H](C[C@H]2[C@H](C[C@H](C(=O)NCC(C)(C)C(N)=O)C(C)C)OCN2C(=O)OC(C)OC(=O)CCC(=O)OC(C)(C)C)C(C)C)ccc1OC]

The compound was prepared using a method analogous to the method described in Example 49 from succinic acid mono tert-butylate (10 mg), cesium iodide (14 mg), cesium carbonate (as a base, 19 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (35 mg) to give 32 mg of desired product as colorless oil. LCMS: 794.5 [M+1]⁺, 816.5 [M+Na]⁺

### Example 58

### 1-({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methoxy)-4-oxobutanoic acid

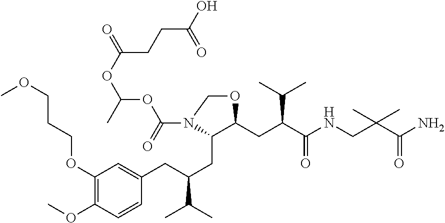 [SMILES: COCCCOc1cc(C[C@@H](C[C@H]2[C@H](C[C@H](C(=O)NCC(C)(C)C(N)=O)C(C)C)OCN2C(=O)OC(C)OC(=O)CCC(=O)O)C(C)C)ccc1OC]

The compound was prepared using a method analogous to the method described in Example 50 from 1-{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}ethyl tert-butyl butanedioate (32 mg) to give 25 mg of desired product as slightly rose oil. LCMS: 738.5 [M+1]⁺, 760.5 [M+Na]⁺; 736.4 [M−1]⁻

### Example 59

### 1-{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}ethyl tert-butyl (2E)-but-2-enedioate

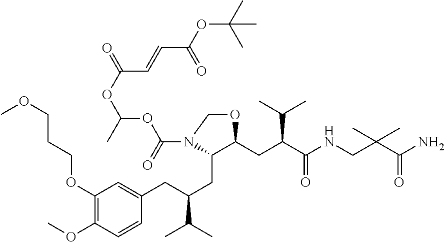 [SMILES: COCCCOc1cc(C[C@@H](C[C@H]2[C@H](C[C@H](C(=O)NCC(C)(C)C(N)=O)C(C)C)OCN2C(=O)OC(C)OC(=O)/C=C/C(=O)OC(C)(C)C)C(C)C)ccc1OC]

The compound was prepared using a method analogous to the method described in Example 49 from fumaric acid mono tert-butylate (27 mg), cesium iodide (35 mg), cesium carbonate (as a base, 51 mg), and 1-chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)-benzyl]-3-methylbutyl}-oxazolidine-3-carboxylate (90 mg) to give 60 mg of desired product as colorless oil. LCMS: 806.5 [M+1]⁺, 828.4 [M+Na]⁺

### Example 60

### 1-({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}ethoxy)oxo-(2E)-but-2-enoic acid

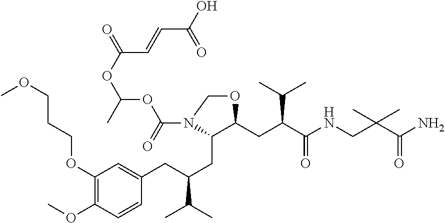 [SMILES: COCCCOc1cc(C[C@@H](C[C@H]2[C@H](C[C@H](C(=O)NCC(C)(C)C(N)=O)C(C)C)OCN2C(=O)OC(C)OC(=O)/C=C/C(=O)O)C(C)C)ccc1OC]

The compound was prepared using a method analogous to the method described in Example 50 from 1-{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}ethyl tert-butyl (2E)-but-2-enedioate (60 mg) to give 35.1 mg of desired product as slightly rose oil. LCMS: 750.4 [M+1]⁺, 772.4 [M+Na]⁺; 748.4 [M−1]⁻

### Example 61

### (1-methylpiperidin-4-yl)methyl (4S,5S)-5-((2S)-2-{[(3-amino-2,2-dimethyl-3-oxopropyl)amino]carbonyl}-3-methylbutyl)-4-[(2S)-2-[4-methoxy-3-(3-methoxypropoxy)benzyl]-3-methylbutyl]-1,3-oxazolidine-3-carboxylate

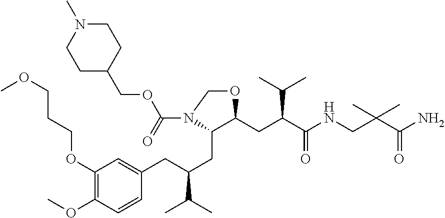 [SMILES: COCCCOc1cc(C[C@@H](C[C@H]2[C@H](C[C@H](C(=O)NCC(C)(C)C(N)=O)C(C)C)OCN2C(=O)OCC2CCN(C)CC2)C(C)C)ccc1OC]

To a solution of 1-methyl-4-piperidinemethanol (23 mg) in DCM, was added bis(trichloromethyl) carbonate (30 mg), followed by DMAP (73 mg); and the resulting milky suspension was stirred at r.t. for 15 min. Then a solution of (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine (100 mg) in DCM was added and the resulting clear solution was stirred for 3 h at r.t. Reaction mixture was concentrated by rotary evaporation and purified by preparative HPLC to give 36 mg of desired product as red-brow oil. LCMS: 719.5 [M+1]⁺, 741.5 [M+Na]⁺; 717.6 [M−1]⁻

### Example 62

### {[(1-hydroxycyclopropyl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

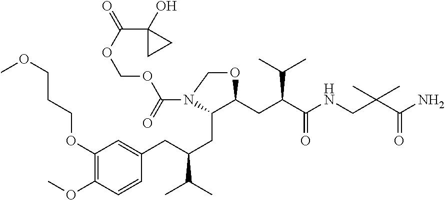 [SMILES: COCCCOc1cc(C[C@@H](C[C@H]2[C@H](C[C@H](C(=O)NCC(C)(C)C(N)=O)C(C)C)OCN2C(=O)OCOC(=O)C2(O)CC2)C(C)C)ccc1OC]

The compound was prepared using a method analogous to the method described in Example 18 from 1-hydroxy-1-cyclopropanecarboxylic acid (6 mg), cesium iodide (14 mg), cesium carbonate (17 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (30 mg) to give 24 mg of desired product as white foam after purification by column chromatography on YMC silica gel (EtOAc with 5% MeOH). LCMS: 722.5 [M+1]⁺, 744.5 [M+Na]⁺

### Example 63

### 1-{[(1-methyl-1H-imidazol-5-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

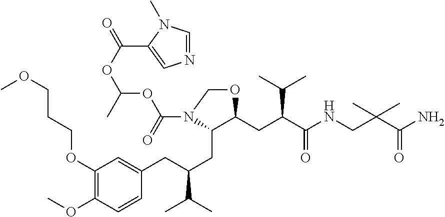 [SMILES: COCCCOc1cc(C[C@@H](C[C@H]2[C@H](C[C@H](C(=O)NCC(C)(C)C(N)=O)C(C)C)OCN2C(=O)OC(C)OC(=O)c2cncn2C)C(C)C)ccc1OC]

The compound was prepared using a method analogous to the method described in Example 33 from 1-methyl-1H-imidazole-2-carboxylic acid (42 mg), cesium iodide (43 mg), cesium carbonate (as a base, 88 mg), and 1-chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (100 mg) to give 25 mg of desired product as colorless oil. LCMS: 760.5 [M+1]⁺, 782.5 [M+Na]⁺; 758.5 [M−1]⁻

### Example 64

### 2-Chloroethyl (4S,5S)-5-((2S)-2-{[(3-amino-2,2-dimethyl-3-oxopropyl)amino]carbonyl}-3-methylbutyl)-4-[(2S)-2-[4-methoxy-3-(3-methoxypropoxy)benzyl]-3-methylbutyl]-oxazolidine-3-carboxylate

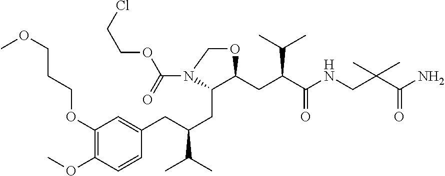 [SMILES: COCCCOc1cc(C[C@@H](C[C@H]2[C@H](C[C@H](C(=O)NCC(C)(C)C(N)=O)C(C)C)OCN2C(=O)OCCCl)C(C)C)ccc1OC]

The compound was prepared using a method analogous to the method described in Example 13 from Aliskiren (300 mg) and 2-chloroethyl chloroformate (60 μl) to give 121 mg of desired product as white foam. LCMS: 670.5 [M+1]⁺, 792.5 [M+Na]⁺; 668.4 [M−1]⁻

### Example 65

### 2-[(1,3-Dioxan-5-ylcarbonyl)oxy]ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

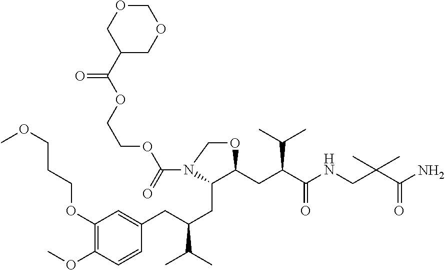 [SMILES: COCCCOc1cc(C[C@@H](C[C@H]2[C@H](C[C@H](C(=O)NCC(C)(C)C(N)=O)C(C)C)OCN2C(=O)OCCOC(=O)C2COCOC2)C(C)C)ccc1OC]

1,3-Dioxane-5-carboxylic acid (20 mg), cesium carbonate (49 mg), cesium iodide (21 mg) and 2-chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-oxazolidine-3-carboxylate (55 mg) were mixed with dry DMF (1 ml) heated in a closed vial at 80° C. for 18 h at stirring. Reaction mixture was then cooled down and acidified with 10% citric acid, mixed with water and brine and extracted into DCM (4×20 ml). Combined organic extracts were washed with brine, dried over MgSO₄, filtered and concentrated by rotary evaporation to give crude product as oil, which was kept under high vacuum to remove residual DMF. Purified by column chromatography on YMC silica gel (EtOAc then EtOAc/MeOH 9:1) gave about 35 mg of product as colorless oil/white foam. LCMS: 766.5 [M+1]⁺, 788.5 [M+Na]⁺

### Example 66

### 2-{[(1-Methyl-1H-imidazol-5-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

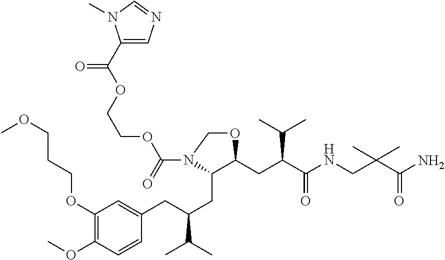 [SMILES: COCCCOc1cc(C[C@@H](C[C@H]2[C@H](C[C@H](C(=O)NCC(C)(C)C(N)=O)C(C)C)OCN2C(=O)OCCOC(=O)c2cncn2C)C(C)C)ccc1OC]

The compound was prepared using a method analogous to the method described in Example 65 from 2-chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-oxazolidine-3-carboxylate (55 mg), cesium carbonate (57 mg), cesium iodide (41 mg) and 3-methyl-3H-imidazole-4-carboxylic acid (21 mg) to give 35 mg of desired product as colorless oil. LCMS: 760.5 [M+1]⁺, 782.5 [M+Na]⁺; 758.6 [M−1]⁻

### Example 67

### 1-(Pyridin-3-yloxy)ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

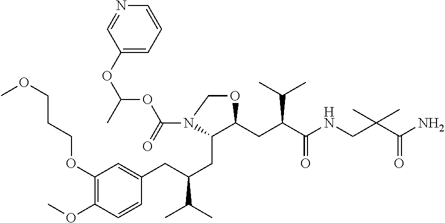 [SMILES: COCCCOc1cc(C[C@@H](C[C@H]2[C@H](C[C@H](C(=O)NCC(C)(C)C(N)=O)C(C)C)OCN2C(=O)OC(C)Oc2cccnc2)C(C)C)ccc1OC]

1-Chloroethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)-benzyl]-3-methylbutyl}-oxazolidine-3-carboxylate (51 mg), cesium carbonate (58 mg) and 3-hydroxypyridine (11 mg) were mixed with dry DMF (1 ml) and stirred overnight at room temperature. Reaction mixture was then mixed with 10% citric acid and brine and extracted into DCM.

Combined organic extracts were washed with brine and dried over magnesium sulfate. Crude material after concentration of organic extract was purified by column chromatography on silica gel (EtOAc) to afford 32 mg of product as colorless oil. LCMS: 729.5 [M+1]⁺, 751.5 [M+Na]⁺; 727.5 [M−1]⁻

### Example 68

### {[(2-Methylpyridin-3-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

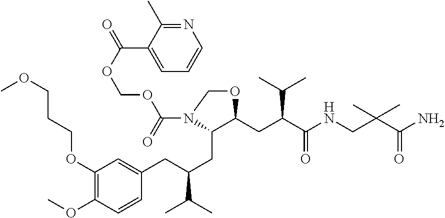 [SMILES: COCCCOc1cc(C[C@@H](C[C@H]2[C@H](C[C@H](C(=O)NCC(C)(C)C(N)=O)C(C)C)OCN2C(=O)OCOC(=O)c2cccnc2C)C(C)C)ccc1OC]

The compound was prepared using a method analogous to the method described in Example 18 from 2-methylpyridine-3-carboxylic acid (20 mg), cesium iodide (24 mg), cesium carbonate (53 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (51 mg) to give 41 mg of desired product as colorless oil. LCMS: 757.5 [M+1]⁺, 779.5 [M+Na]⁺; 755.6 [M−1]⁻

### Example 69

### {[(3-Methylpyridin-2-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

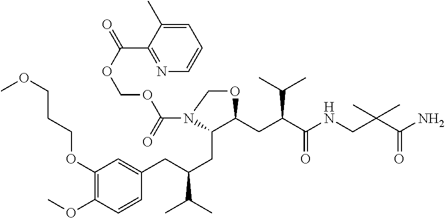 [SMILES: COCCCOc1cc(C[C@@H](C[C@H]2[C@H](C[C@H](C(=O)NCC(C)(C)C(N)=O)C(C)C)OCN2C(=O)OCOC(=O)c2ncccc2C)C(C)C)ccc1OC]

The compound was prepared using a method analogous to the method described in Example 18 from 3-methylpicolinic acid (18 mg), cesium iodide (30 mg), cesium carbonate (54 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) to give 32 mg of desired product as colorless oil. LCMS: 757.5 [M+1]⁺, 779.5 [M+Na]⁺; 755.6 [M−1]⁻

### Example 70

### ({[1-(Hydroxymethyl)cyclopropyl]carbonyl}oxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

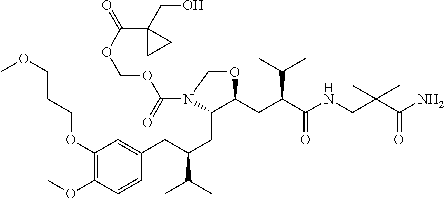 [SMILES: COCCCOc1cc(C[C@@H](C[C@H]2[C@H](C[C@H](C(=O)NCC(C)(C)C(N)=O)C(C)C)OCN2C(=O)OCOC(=O)C2(CO)CC2)C(C)C)ccc1OC]

The compound was prepared using a method analogous to the method described in Example 18 from 1-hydroxymethyl-1-cyclopropanecarboxylic acid (12 mg), cesium iodide (28 mg), cesium carbonate (35 mg), and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (62 mg) to give 51 mg of desired product as colorless oil after purification by column chromatography on YMC silica gel (EtOAc with 5% MeOH). LCMS: 736.5 [M+1]⁺, 758.5 [M+Na]⁺; 734.6 [M−1]⁻

### Example 71

### Pyridine-3-ylmethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

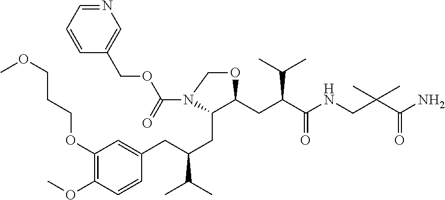 [SMILES: COCCCOc1cc(C[C@@H](C[C@H]2[C@H](C[C@H](C(=O)NCC(C)(C)C(N)=O)C(C)C)OCN2C(=O)OCc2cccnc2)C(C)C)ccc1OC]

Sodium hydride (5 mg, 60% in mineral oil) was mixed with absolute DMF (1.5 ml) and stirred for 10 min, then 3-pyridinemethanol (18 μl) was added to the reaction mixture and stirred for additional 20 min. Then chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (50 mg) was added as DMF solution (1.5 ml) to the reaction mixture and stirred overnight at r.t. Reaction mixture was acidified with 10% citric acid and extracted into DCM. Combined organic extract was washed with brine, dried over magnesium sulfate and concentrated by rotary evaporation. Crude material was purified by column chromatography on silica gel (EtOAc) to give 25 mg of desired compound as colorless oil. LCMS: 699.5 [M+1]⁺, 721.4 [M+Na]⁺; 697.5 [M−1]⁻

### Example 72

### {[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methyl N-pentanoyl-N-{[2′-(1H-tetrazol-5-yl)biphenyl-4-yl]methyl}-L-valinate

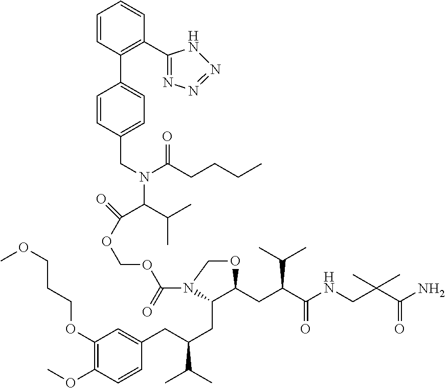 [SMILES: CCCCC(=O)N(Cc1ccc(-c2ccccc2-c2nnn[nH]2)cc1)C(C(=O)OCOC(=O)N1CO[C@@H](C[C@H](C(=O)NCC(C)(C)C(N)=O)C(C)C)[C@@H]1C[C@H](Cc1ccc(OC)c(OCCCOC)c1)C(C)C)C(C)C]

The compound was prepared using a method analogous to the method described in Example 8 from (5)-2-{N-[(2′-(1H-tetrazol-5-yl)biphenyl-4-yl)methyl]pentanamido}-3-methylbutanoic acid (22 mg), cesium carbonate (15 mg), cesium iodide (12 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (30 mg) to give 10 mg of desired product as white foam after purification by column chromatography on YMC silica gel (EtOAc with 5% MeOH). LCMS: 1055.7 [M+1]⁺, 1077.6 [M+Na]⁺; 1053.8 [M−1]⁻

### Example 73

### {[(4-Methyloxazol-5-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate

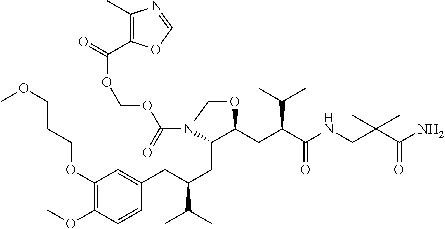 [SMILES: COCCCOc1cc(C[C@@H](C[C@H]2[C@H](C[C@H](C(=O)NCC(C)(C)C(N)=O)C(C)C)OCN2C(=O)OCOC(=O)c2ocnc2C)C(C)C)ccc1OC]

The compound was prepared using a method analogous to the method described in Example 31 from 4-methyloxazole-5-carboxylic acid (17 mg), cesium carbonate (66 mg), cesium iodide (30 mg) and chloromethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate (71 mg) to give 40 mg of desired product as white foam after purification by column chromatography on YMC silica gel (EtOAc with 5% MeOH). LCMS: 747.5 [M+1]⁺, 769.5 [M+Na]⁺; 745.4 [M−1]⁻

### Chemical Stability Studies

The compounds of the invention were studied for their chemical stability:

Stability at pH 2.0:

10 mM DMSO solution of the compound of the formula (I) was prepared. 10 μl of the solution was added to 1 ml of pH 2 aqueous buffer at 37° C. The solution was kept at 37° C. At each time point, a small aliquote was taken and the sample was analyzed by LCMS.

Stability at pH 7.4:

10 mM DMSO solution of the compound of the formula (I) was prepared. 10 μl of the solution was added to 1 ml of pH 7.4 aqueous phosphate buffer at 37° C. The solution was kept at 37° C. At each time points, a small aliquote was taken and the sample was analyzed by LCMS.

### Stability Study in Human Plasma

10 mM solution of the compound of formula (I) in DMSO was added to human plasma (pooled), making the final compound concentration of 20 uM. The sample was incubated at 37° C. At each time point, an aliquot of 100 μl was taken. The aliquot was kept on ice and to it was added 200 μl acetonitrile immediately. After mixing for a few seconds, the sample was centrifuged at 30000 rpm at 10° C. for 7 min. The supernatant was taken and analyzed by LCMS.

### In Vivo Bioavailability Experiment

The following study was employed to investigate the oral bioavailability of representative compounds of the invention by measuring the plasma concentration of aliskiren following a single dose of the compounds of the invention. For comparison, aliskiren hemifumarate was dosed both i.v. (intravenous) and orally. For all experiment, the compounds were administrated to three individually weighted male Sprague-Dawley rats. For all po dosing, the dose was 25 μmole/kg in a dose volume of 8 ml/kg and the dose vehicle was 50% propylene glycol/50% pH 4.75 buffer (0.1M aqueous buffer of NaOAc/HOAc) by volume ratio. For i.v. dosing, aliskiren hemi-fumarate was dosed at 5 umole/kg (calculated based on free base) in a volume of 1.5 ml/kg and the vehicle was saline. For the i.v. dosing of the compounds of invention, a vehicle of 45% PEG400/55% pH 4.75 buffer (0.1M aqueous buffer of NaOAc/HOAc) by volume ratio was used and the dose were 5 μmole/kg in a dose volume of 1.5 ml/kg. Male Sprague-Dawley rats were fasted for about 16-17 h before po dosing and fasting lasted about 2-3 h post-dose. Water was given ad libitum. The blood samples were taken at different time points up to 24 h. For i.v. dosing group, at 5 min, 15 min, 30 min, 1 h, 2 h, 4 h, 6 h, 8 h, 24 h. For oral doing group, at 15 min, 30 min, 1 h, 2 h, 4 h, 611, 24 h. The blood samples were collected in heparinized tube and centrifuged at 3500 rpm for 5 min. The plasma samples were stored at about −20° C. for analysis.

After the work-up of the plasma samples, LC-MS/MS was used for the quantitation of the aliskiren and the compounds of the invention. Standard curves were made for aliskiren and the substances for study. The lowest LOQ (limit of quantitation) for aliskiren in plasma was 0.5 ng/25

### Results of Some Compounds of the Invention

TABLE 1
Chemical stability of the compounds

| Compound | pH 2 | pH 7.4 |
|---|---|---|
| Aliskiren | A | A |
| Example 6 | A | B |
| Example 12 | A | B |
| Example 27 | A | A |
| Example 32 | A | B |
| Example 35 | A | A |
| Example 43 | A | B |
| Example 44 | A | A |
| Example 46 | C | A |
| Example 53 | A | C |

A: Less than 5% decomposition after 3 h incubation at 37° C.
B: Less than 25% decomposition after 3 h incubation at 37° C.
C: Less than 50% decomposition after 3 h incubation at 37° C.

TABLE 2
In vivo bioavailability study after iv and
oral dosing of the compounds in rats

| | AUC₀₋ₜ (h* ng/ml) |
|---|---|
| Dosed Compounds | of aliskiren |
| Aliskiren hemifumarate i.v. | 994 ± 162 |
| (5 μmole/kg) | |
| Aliskiren hemifumarate p.o. | 28.0 ± 20.5 |
| (25 μmole/kg) | |
| Example 6 p.o. (25 μmole/kg) | A |
| Example 12 p.o. (25 μmole/kg) | A |
| Example 27 p.o. (25 μmole/kg) | B |
| Example 32 p.o. (25 μmole/kg) | B |
| Example 35 p.o. (25 μmole/kg) | B |
| Example 43 p.o. (25 μmole/kg) | B |
| Example 44 p.o. (25 μmole/kg) | C |
| Example 46 p.o. (25 μmole/kg) | A |
| Example 53 p.o. (25 μmole/kg) | A |
| Example 63 p.o. (25 μmole/kg) | C |

A: AUC₀₋ₜ (h* ng/ml) of aliskiren is between 40 and 100
B: AUC₀₋ₜ (h* ng/ml) of aliskiren is between 100 and 300
C: AUC₀₋ₜ (h* ng/ml) of aliskiren is > 300

1. A compound of formula (I) 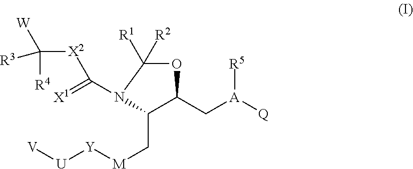
wherein
R¹ and R² independently represent
H, C₁-C₆alkyl, C₃-C₆cycloalkyl or C₃-C₆cycloalkyl-C₁-C₃alkyl, wherein said C₁-C₆alkyl, C₃-C₆cycloalkyl or C₃-C₆cycloalkyl-C₁-C₃alkyl is optionally substituted by one or more substituents independently selected from halogen, CN, NH(C₁-C₆alkyl), N(C₁-C₆alkyl)₂, C₁-C₆alkyl and C₁-C₆alkoxy;
or R¹ and R² together with the carbon to which they are bonded form
a C₃-C₆cycloalkyl or a 4-6 membered heterocyclyl, wherein said C₃-C₆cycloalkyl or 4-6 membered heterocyclyl is optionally substituted by one or more substituents independently selected from halogen, CN, NH(C₁-C₆alkyl), N(C₁-C₆alkyl)₂, C₁-C₆alkyl and C₁-C₆alkoxy;
R³ and R⁴ independently represent
H, C₁-C₈alkyl, C₂-C₈alkenyl, C₂-C₈alkynyl, C₃-C₆cycloalkyl, C₃-C₆cycloalkyl-C₁-C₆alkyl, C₁-C₈alkoxy, C₁-C₈alkoxy-C₁-C₆alkyl, aryl-C₁-C₆alkyl, heterocyclyl-C₁-C₆alkyl, aryl, aryloxy, heterocyclyl or heterocyclyloxy, wherein said C₁-C₈alkyl, C₂-C₈alkenyl, C₂-C₈alkynyl, C₃-C₆cycloalkyl, C₃-C₆cycloalkyl-C₁-C₆alkyl, C₁-C₈alkoxy, C₁-C₈alkoxy-C₁-C₆alkyl, aryl-C₁-C₆alkyl, heterocyclyl-C₁-C₆alkyl, aryl, aryloxy, heterocyclyl or heterocyclyloxy is optionally substituted by one or more substituents independently selected from halogen, OH, CN, NO₂, NH₂, NH(C₁-C₆alkyl), N(C₁-C₆alkyl)₂, C₁-C₆alkyl, C₁-C₆alkoxy and C₃-C₆cycloalkyl;
or R³ and R⁴ together with the carbon to which they are bonded form
a C₃-C₈cycloalkyl or a 4-8 membered heterocyclyl, wherein said C₃-C₈cycloalkyl or 4-8 membered heterocyclyl is optionally substituted by one or more substituents independently selected from halogen, OH, CN, NO₂, NH₂, NH(C₁-C₃alkyl), N(C₁-C₃alkyl)₂, C₁-C₃alkyl, C₃-C₆cycloalkyl and C₁-C₃alkoxy;
X¹ represents
O or S;
X² represents
O or S;
W represents
H, R⁶X¹—, C₂-C₆alkyl, halogen, (OH)₂P(O)O, [R^{a}C(O)OCH₂O]₂P(O)O, or [R^{a}C(O)OCH(C₁-C₃alkyl)O]₂P(O)O, [R^{a}C(O)SCH₂CH₂O]₂P(O)O;
R^{a} represents
C₁-C₆alkyl, C₃-C₆cycloalkyl, C₂-C₆-alkenyl, heterocyclyl or aryl, wherein said C₁-C₆alkyl, C₃-C₆cycloalkyl, C₂-C₆-alkenyl, heterocyclyl or aryl is optionally substituted by one or more substituents independently selected from halogen, OH, NH₂, NH(C₁-C₃-alkyl), N(C₁-C₃-alkyl)₂, C₁-C₃alkyl, C₁-C₃alkoxy, aryl and heterocyclyl;
R⁶ represents
—C(═X¹)TZ;
T represents
O, S, NH, N(C₁-C₃alkyl) or a single bond;
Z represents
C₁-C₁₈alkyl, C₂-C₁₈alkenyl, C₂-C₁₈alkynyl, C₃-C₈cycloalkenyl, C₄-C₈cycloalkynyl, aryl, heterocyclyl, C₃-C₈cycloalkyl, C₁-C₁₈alkyl-heterocyclyl, tetrazolyl-biphenyl-methyl-heterocyclyl, tetrazolyl-biphenyl-methyl-heterocyclylmethyl, tetrazolyl-biphenyl-methyl-amino-C₁-C₆alkyl, oxadiazolyl-biphenyl-methyl-heterocyclyl, heterocyclylmethyl-aryl, C₁-C₆alkyl-aryl or C₁-C₆alkyl-C₃-C₈cycloalkyl, wherein said C₁-C₁₈alkyl, C₂-C₁₈alkenyl, C₂-C₁₈alkynyl, C₃-C₈cycloalkenyl, C₄-C₈cycloalkynyl, aryl, heterocyclyl, C₃-C₈cycloalkyl, C₁-C₁₈alkyl-heterocyclyl, tetrazolyl-biphenyl-methyl-heterocyclyl, tetrazolyl-biphenyl-methyl-heterocyclylmethyl, tetrazolyl-biphenyl-methyl-amino-C₁-C₆alkyl, oxadiazolyl-bephenyl-methyl-heterocyclyl, heterocyclylmethyl-aryl, C₁-C₆alkyl-aryl or C₁-C₆alkyl-C₃-C₈cycloalkyl is optionally substituted by one or more substituents independently selected from halogen, OH, CN, oxo, N₃, NO₂, NH₂, NH(C₁-C₆alkyl), N(C₁-C₆alkyl)₂, C₁-C₆alkanoylNH, C₂-C₆alkoxycarbonylNH, C₁-C₆alkanoyl, C₁-C₆alkanoyloxy, COOH, (OH)₂P(O)O, [R^{a}C(O)OCH₂O]₂P(O)O, [R^{a}C(O)OCH(C₁-C₃alkyl)O]₂P(O)O, [R^{a}C(O)SCH₂CH₂O]₂P(O)O, NH₂C(O), C₁-C₆alkyl, C₂-C₆alkenyl, C₂-C₆alkynyl, C₁-C₆alkoxy, C₁-C₆alkoxycarbonyl, C₁-C₆alkoxycarbonylNH, NH₂C₁-C₆alkyl, C₁-C₆alkoxycarbonylNHC₁-C₃alkyl, arylC₁-C₄alkylcarbonylNH, C₃-C₆cycloalkyl, C₃-C₆cycloalkenyl, C₃-C₆cycloalkoxy, C₃-C₆cycloalkenyloxy, C₁-C₃alkoxy-C₁-C₆alkoxy, aryl, aryloxy, heterocyclyloxy and heterocyclyl;
M represents
O, S, SO₂, N(R⁷) or 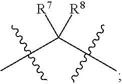
R⁷ represents
H, C₁-C₆alkyl, C₂-C₆alkenyl, C₂-C₆alkynyl, C₃-C₆cycloalkyl, aryl, heterocyclyl or aryl(C₁-C₆)alkyl, wherein said C₁-C₆alkyl, C₂-C₆alkenyl, C₂-C₆alkynyl, C₃-C₆cycloalkyl, aryl, heterocyclyl or aryl(C₁-C₆)alkyl is optionally substituted by one or more substituents independently selected from halogen, C₃-C₆cycloalkyl or C₁-C₆alkyl, wherein said C₃-C₆cycloalkyl or C₁-C₆alkyl is optionally substituted by one or more substituents selected from halogen, aryl and heterocyclyl;
R⁸ represents
H, OH, halogen, C₁-C₆alkyl or C₁-C₆alkoxy;
or R⁷ and R⁸ together with the carbon atom to which they are bonded form a C₃-C₈cycloalkyl;
Y represents
a single bond, CH₂, C₂-C₆alkanoyloxymethylene, O, S, SO, SO₂, NH, N(C₁-C₄alkyl), C(O), or CH(OH);
U represents
a single bond, CH₂, C(O), C(O)NH, NHC(O), NH or N(C₁-C₄alkyl);
V represents
a 3-18-membered saturated, partially unsaturated or aromatic mono-, bi- or tricyclic system, said system is a carbocyclic ring system or a heterocyclic ring system selected from C₃-C₁₂cycloalkyl, C₃-C₁₂cycloalkenyl, C₄-C₁₂cycloalkynyl, heterocyclyl and aryl, wherein said system is optionally substituted with one, two, three or four substituents independently selected from halogen, OH, CN, oxo, COOH, CF₃, NO₂, NH₂, NH(C₁-C₆alkyl), N(C₁-C₆alkyl)₂, C₁-C₆alkoxy, C₁-C₆alkoxy-C₁-C₆alkoxy, NH₂C(O), C₃-C₆cycloalkyl, C₂-C₆alkenyl, C₃-C₆cycloalkoxy-C₁-C₆alkoxy, C₃-C₈cycloalkyl-C₁-C₆alkoxy, dioxalanyl, hydroxyl-C₂-C₇alkoxy, haloC₂-C₇alkoxy, carbamoyloxy-C₂-C₇alkoxy, [(C₅H₅N)NHC(O)]C₁-C₇alkoxy, C₃-C₆cycloalkoxy, C₂-C₇alkenyloxy, C₁-C₆alkanoyloxy, C₁-C₆alkoxycarbonyl, C₁-C₃alkoxycarbonyl, C₁-C₆alkylenedioxy, aryl, phenoxy, phenylthio, pyridyl and C₁-C₆alkyl, wherein said C₁-C₆alkyl is optionally substituted by C₃-C₆cycloalkoxy, C₁-C₆alkoxy, (C₅H₅N)C(O)NH, NH₂C(O), NH(C₁-C₃alkyl)C(O), N(C₁-C₃)₂C(O), NH₂C(O)C₁-C₃alkoxy, NH(C₁-C₃alkyl)C(O)C₁-C₃alkoxy, N(C₁-C₃alkyl)₂C(O)C₁-C₃alkoxy or phenyl;
A represents
CH or N;
R⁵ represents
H, C₁-C₆alkyl, C₂-C₆alkenyl, C₂-C₆alkynyl, C₃-C₆cycloalkyl or C₁-C₆alkoxy, wherein said C₁-C₆alkyl, C₂-C₆alkenyl, C₂-C₆alkynyl, C₃-C₆cycloalkyl or C₁-C₆alkoxy is optionally substituted by one or more of substituents independently selected from halogen, OH, C₃-C₆cycloalkyl, C₁-C₆alkoxy and aryl;
Q represents
C₁-C₈alkyl, C₃-C₈cycloalkyl, NH(C₁-C₈alkyl)C(O)C₁-C₆alkyl, N(C₁-C₈alkyl)₂C(O)C₁-C₆alkyl, aryl, heterocyclyl or heterocyclyl-C₁-C₄alkyl;
or Q is selected from the group of partial structures consisting of E1 and E2 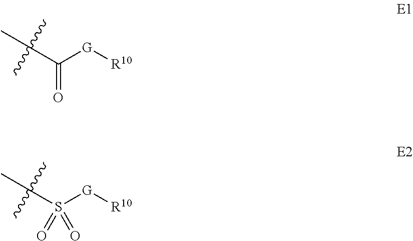
G represents 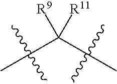
O, or N(R⁹);
R¹¹ represents
H or C₁-C₆alkyl;
or R⁵, Q and A, wherein A is N, form a 3-18-membered saturated, partially unsaturated or aromatic mono-, bi- or tricyclic ring system, wherein said system is optionally substituted by one, two, three or four substituents independently selected from halogen, OH, oxo, CN, C₁-C₆alkyl, C₃-C₈cycloalkyl, C₃-C₈cycloalkanoyl, C₁-C₈alkanoyl, aryl-C₁-C₆alkanoyl, C₁-C₈alkoxycarbonyl, C₁-C₈alkyl-SO₂, heterocyclylSO₂, aryl and heterocyclyl;
R⁹ represents
H, C₁-C₆alkyl, C₃-C₈cycloalkyl, C₂-C₆alkenyl or C₁-C₆alkoxy, wherein said C₁-C₆alkyl, C₃-C₈cycloalkyl, C₂-C₆alkenyl or C₁-C₆alkoxy is optionally substituted by one or more halogen;
R¹⁰ represents
H, C₁-C₁₂alkyl, C₂-C₁₂alkenyl, C₃-C₁₂cycloalkyl, C₃-C₁₂cycloalkenyl, heterocyclyl or aryl, wherein said C₁-C₁₂alkyl, C₂-C₁₂alkenyl, C₃-C₁₂cycloalkyl, C₃-C₁₂cycloalkenyl, heterocyclyl or aryl is optionally substituted by one or more substituents independently selected from halogen, OH, CN, NO₂, C₁-C₈alkoxy, C₃-C₆cycloalkyl, aryloxy, heterocycloxy, NH₂C(O), NH(C₁-C₈alkyl), NH(aryl), NH(heterocyclyl), NH(aryl)C(O), NH(heterocyclyl)C(O), C₁-C₈alkyl-C(O)NH, arylC(O)NH, C₁-C₈alkanoyl, C₁-C₆alkoxyC(O), C₁-C₈alkylSO₂, aryl-SO₂, aryl and heterocyclyl;
or R¹⁰ is
C₁-C₈alkyl or C₁-C₈alkenyl, wherein said C₁-C₈alkyl or C₁-C₈alkenyl is optionally substituted by NH₂C(O), NH(C₁-C₈alkyl)C(O), NH(C₃-C₈cycloalkyl)C(O), NH(C₃-C₆-alkenyl)C(O), N(C₁-C₆alkyl)₂C(O), C₁-C₆alkoxycarbonylNHC(O), N(C₃-C₈cycloalkyl)₂C(O), N(C₃-C₆cycloalkyl)(C₁-C₃alkyl)C(O), N(heterocyclyl)(C₁-C₆alkyl)C(O), NH₂C(S) or NH(C₁-C₈alkyl)C(S);
or R¹⁰ is
C₁-C₆alkyl or C₂-C₆alkenyl, wherein said C₁-C₆alkyl or C₂-C₆alkenyl is optionally substituted with NH₂C(O)C₃-C₆cycloalkyl;
or R⁹ and R¹⁰ together with the atom of G to which R⁹ and R¹⁰ are bonded form
a 3-18-membered saturated, partially unsaturated or aromatic mono-, bi- or tricyclic system, said system is a carbocyclic ring system or a heterocyclic ring system, wherein said system is optionally substituted by one, two, three or four substituents independently selected from halogen, OH, oxo, C₁-C₆alkyl, C₃-C₈cycloalkyl, C₁-C₆alkoxy, C₃-C₈cycloalkoxy, C₁-C₈alkanoyl, C₁-C₈alkanoyloxy, aryl-C₁-C₆alkanoyl, C₁-C₈alkoxycarbonyl, C₁-C₈alkyl-SO₂, heterocyclyl-SO₂, aryl and heterocyclyl;
with the proviso that R⁴ is not aryl when R³ and W are H;
and with the proviso that R³ is not aryl when R⁴ and W are H;
or a pharmaceutically acceptable salt thereof.

2. A compound according to claim 1, wherein
Z represents
C₁-C₁₈alkyl, C₂-C₁₈alkenyl, C₂-C₁₈alkynyl, C₃-C₈cycloalkenyl, C₄-C₈cycloalkynyl, aryl, heterocyclyl or C₃-C₈cycloalkyl, wherein said C₁-C₁₈alkyl, C₂-C₁₈alkenyl, C₂-C₁₈alkynyl, C₃-C₈cycloalkenyl, C₄-C₈cycloalkynyl, aryl, heterocyclyl or C₃-C₈cycloalkyl is optionally substituted by one or more of the substituents independently selected from: halogen, OH, CN, oxo, N₃, NO₂, NH₂, NH(C₁-C₆alkyl), N(C₁-C₆alkyl)₂, C₁-C₆alkanoylNH, C₂-C₆alkoxycarbonylNH, C₁-C₆-alkanoyl, C₁-C₆alkanoyloxy, COOH, (OH)₂P(O)O, [R^{a}C(O)OCH₂O]₂P(O)O, [R^{a}C(O)OCH(C₁-C₃alkyl)O]₂P(O)O, [R^{a}C(O)SCH₂CH₂O]₂P(O)O, NH₂C(O), C₁-C₆alkyl, C₂-C₆alkenyl, C₂-C₆alkynyl, C₁-C₆alkoxy, C₁-C₆alkoxycarbonyl, C₃-C₆cycloalkyl, C₃-C₆cycloalkenyl, C₃-C₆cycloalkoxy, C₃-C₆cycloalkenyloxy, C₁-C₃alkoxy-C₁-C₆alkoxy, aryl, aryloxy, heterocyclyloxy and heterocyclyl.

3. A compound according to claim 1, wherein
X¹ is O;
X² is O or S; and
W is R⁶O—.

4. A compound according to claim 3, wherein
X² is O.

5. A compound according to claim 1, wherein
X¹ is O;
X² is O;
M is 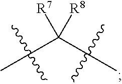
and
U is a single bond.

6. A compound according to claim 1, wherein
X¹ is O;
X² is O;
W is R⁶O—;
M is 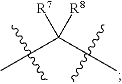
U is a single bond;
A is CH and
Q is E1.

7. A compound according to claim 1, wherein
R⁵ is
C₁-C₆alkyl or C₃-C₆cycloalkyl.

8. A compound according to claim 1, wherein
V—Y—U-M is: 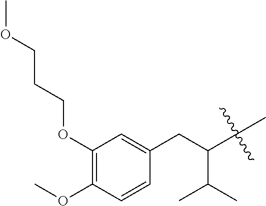 [SMILES: *C(Cc1ccc(OC)c(OCCCOC)c1)C(C)C]
R⁵ is isopropyl;
Q is E1, wherein G is N(R⁹); and
R⁹ is H.

9. A compound according to claim 1, wherein,
V—U—Y-M is 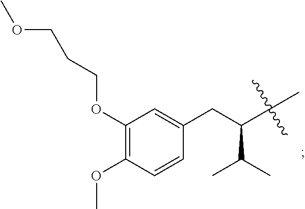 [SMILES: *[C@H](Cc1ccc(OC)c(OCCCOC)c1)C(C)C]
A(R⁵)Q is 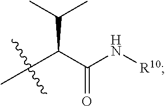
and
R¹⁰ is
C₁-C₆alkyl, NH₂C(O)C₂-C₆alkyl, NH(C₁-C₆alkyl)C(O)C₂-C₅alkyl, N(C₁-C₆alkyl)₂C(O)C₂-C₅alkyl, C₁-C₆alkoxycarbonylNHC(O)—C₂-C₆alkyl, aryl-C₁-C₃alkyl, C₃-C₆cycloalkyl-C₁-C₂alkyl, NH₂C(O)cyclopropyl, C₃-C₆cycloalkyl or aryl.

10. A compound according to claim 1, wherein
V—U—Y-M is 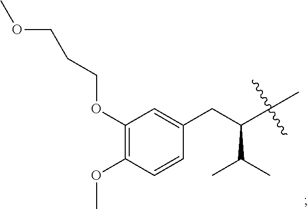 [SMILES: *[C@H](Cc1ccc(OC)c(OCCCOC)c1)C(C)C]
and
A(R⁵)Q is

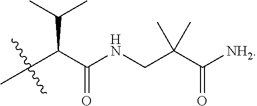 [SMILES: *[C@H](C(=O)NCC(C)(C)C(N)=O)C(C)C]

11. A compound according to claim 1, wherein
X¹ is O;
X² is O;
W is R⁶O—;
V—U—Y-M is 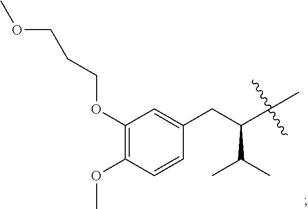 [SMILES: *[C@H](Cc1ccc(OC)c(OCCCOC)c1)C(C)C]
and
A(R⁵)Q is

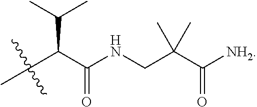 [SMILES: *[C@H](C(=O)NCC(C)(C)C(N)=O)C(C)C]

12. A compound according to claim 1, wherein
V—U—Y-M is 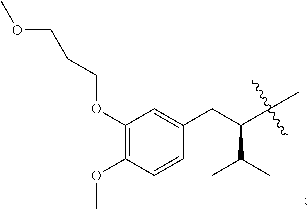 [SMILES: *[C@H](Cc1ccc(OC)c(OCCCOC)c1)C(C)C]
A(R⁵)Q is 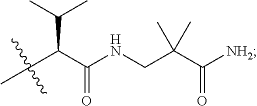 [SMILES: *[C@H](C(=O)NCC(C)(C)C(N)=O)C(C)C]
R¹ and R² independently represent
H, methyl or ethyl;
or R¹ and R² together with the carbon to which they are bonded form
a C₃-C₆cycloalkyl or a 4-6 membered heterocyclyl, wherein said C₃-C₆cycloalkyl or 4-6 membered heterocyclyl is optionally substituted by one or more substituents independently selected from halogen, CN, NH(C₁-C₃alkyl), N(C₁-C₃alkyl)₂, C₁-C₃alkyl and C₁-C₃alkoxy;
R³ and R⁴ independently represent
H or methyl;
or R³ and R⁴ together with the carbon to which they are bonded form
a C₃-C₈cycloalkyl or a 4-8 membered heterocyclyl, wherein said C₃-C₈cycloalkyl or 4-8 membered heterocyclyl is optionally substituted by one or more substituents independently selected from halogen, OH, NH₂, NH(C₁-C₃alkyl), N(C₁-C₃alkyl)₂, C₁-C₃alkyl or C₁-C₃alkoxy;
X¹ is O;
X² is O
W is R⁶O—;
R⁶ is —C(═X¹)TZ
T is a single bond or O; and
Z represents
C₁-C₁₈alkyl, C₂-C₁₈alkenyl, C₂-C₁₈alkynyl, C₃-C₈cycloalkenyl, C₄-C₈cycloalkynyl, aryl, heterocyclyl or C₃-C₈cycloalkyl, wherein said C₁-C₁₈alkyl, C₂-C₁₈alkenyl, C₂-C₁₈alkynyl, C₃-C₈cycloalkenyl, C₄-C₈cycloalkynyl, aryl, heterocyclyl or C₃-C₈cycloalkyl is optionally substituted by one or more of the substituents independently selected from: halogen, OH, CN, oxo, N₃, NO₂, NH₂, NH(C₁-C₆alkyl), N(C₁-C₆alkyl)₂, C₁-C₆alkanoylNH, C₂-C₆alkoxycarbonylNH, C₁-C₆alkanoyl, C₁-C₆alkanoyloxy, COOH, (OH)₂P(O)O, [R^{a}C(O)OCH₂O]₂P(O)O, [R^{a}C(O)OCH(C₁-C₃alkyl)O]₂P(O)O, [R^{a}C(O)SCH₂CH₂O]₂P(O)O, NH₂C(O)—, C₁-C₆alkyl, C₂-C₆alkenyl, C₂-C₆alkynyl, C₁-C₆alkoxy, C₁-C₆alkoxycarbonyl, C₃-C₆cycloalkyl, C₃-C₆cycloalkenyl, C₃-C₆cycloalkoxy, C₃-C₆cycloalkenyloxy, C₁-C₃alkoxy-C₁-C₆alkoxy-, aryl, aryloxy, heterocyclyloxy and heterocyclyl.

13. A compound according to claim 1, wherein
R¹ and R² independently represent
H or C₁-C₂alkyl;
R³ and R⁴ independently represent
H or C₁-C₃alkyl;
X¹ represents O;
X² represents O;
W represents
R⁶X¹— or H;
R⁶ represents
—C(═X¹)TZ;
T represents
O or a single bond;
Z represents
C₁-C₈alkyl, C₂-C₁₈alkenyl, C₃-C₈cycloalkyl, aryl, heterocyclyl, or C₁-C₆alkyl-C₃-C₈cycloalkyl, wherein said C₁-C₈alkyl, C₂-C₁₈alkenyl, C₃-C₈cycloalkyl, aryl, heterocyclyl, C₁-C₆alkyl-aryl or C₁-C₆alkyl-C₃-C₈cycloalkyl is optionally substituted by one or two substituents independently selected from halogen, OH, oxo, NH₂, N(C₁-C₆alkyl)₂, C₂-C₄alkoxycarbonylNH, C₁-C₆alkyl, C₁-C₆alkoxy, C₁-C₆alkoxycarbonylNH, C₁-C₆alkoxycarbonyl, C₃-C₆cycloalkyl, C₁-C₃alkoxy-C₁-C₆alkoxy-, heterocyclyloxy, heterocyclyl, NH₂C₁-C₆alkyl, C₁-C₆alkoxycarbonylNHC₁-C₃alkyl and arylC₁-C₄alkylcarbonylNH;
V—U—Y-M is 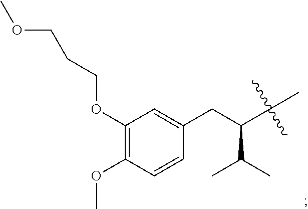 [SMILES: *[C@H](Cc1ccc(OC)c(OCCCOC)c1)C(C)C]
A(R⁵)Q is 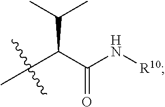
and
R¹⁰ represents C₁-C₄alkyl, wherein said C₁-C₄alkyl is optionally substituted by one NH₂C(O).

14. A compound according to claim 1, wherein
R¹ and R² independently represent
H or C₁-C₂alkyl;
R³ and R⁴ independently represent
H or C₁-C₃alkyl;
X¹ represents O;
X² represents O;
W represents R⁶X¹—;
R⁶ represents —C(═X¹)TZ;
T represents
O or a single bond;
Z represents
C₁-C₁₈alkyl-heterocyclyl, [2′-(1H-tetrazol-5-yl)biphenyl-4-yl]methyl-heterocyclyl, [2′-(1H-tetrazol-5-yl)biphenyl-4-yl]methyl-heterocyclyl-methyl, [2′-(1H-tetrazol-5-yl)biphenyl-4-yl]methylamino-C₁-C₆alkyl, oxadiazolyl-biphenyl-methyl-heterocyclyl or heterocyclylmethyl-biphenyl, wherein said C₁-C₁₈alkyl-heterocyclyl, [2′-(1H-tetrazol-5-yl)biphenyl-4-yl]methyl-heterocyclyl, [2′-(1H-tetrazol-5-yl)biphenyl-4-yl]methyl-heterocyclyl-methyl, [2′-(1H-tetrazol-5-yl)biphenyl-4-yl]methylamino-C₁-C₆alkyl, oxadiazolyl-biphenyl-methyl-heterocyclyl or heterocyclylmethyl-biphenyl is optionally substituted by one or more substituents independently selected from halogen, OH, C₂-C₆alkanoyl, C₁-C₆alkyl, C₁-C₆alkoxy, heterocyclyloxy, hydroxyC₁-C₄alkyl and heterocyclyl;
V—U—Y-M is 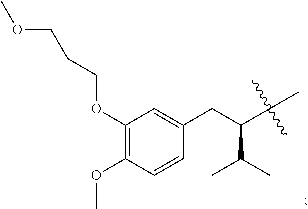 [SMILES: *[C@H](Cc1ccc(OC)c(OCCCOC)c1)C(C)C]
and
A(R⁵)Q is

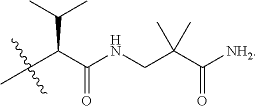 [SMILES: *[C@H](C(=O)NCC(C)(C)C(N)=O)C(C)C]

15. A compound selected from the group consisting of:
(4S,5S)-1-(isobutyryloxy)ethyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S)-pivaloyloxymethyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S)-isobutyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}-oxazolidine-3-carboxylate;
(4S,5S)-valyloxymethyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate, trifluoroacetic acid salt;
(4S,5S)-(ethoxycarbonyloxy)methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S)-(isopropoxycarbonyloxy)methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
{[(2S)-2-hydroxypropanoyl]oxy}methyl (4S,5S)-5-[(2S)-2-(3-amino-2,2-dimethyl-3-oxopropylaminocarbonyl)-3-methylbutyl]-4-{(2S)-2-[4-methoxy-3-(3-methoxypropoxy)benzyl]-3-methylbutyl}-1,3-oxazolidine-3-carboxylate;
{[(2S)-2-(ethoxymethoxy)propanoyl]oxy}methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate;
{(4S,5S)-5-[(2S)-2-(3-amino-2,2-dimethyl-3-oxopropylaminocarbonyl)-3-methylbutyl]-4-{(2S)-2-[4-methoxy-3-(3-methoxypropoxy)benzyl]-3-methylbutyl}-1,3-oxazolidin-3-yl-carbonyloxy}methyl morpholine-4-carboxylate;
(4S,5S) [(pyridine-3-yl)carbonyloxy]methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S) [(pyridine-2-yl)carbonyloxy]methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
[(2-methylpropoxycarbonyl)oxy]methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate;
{[(pyridin-3-ylmethoxy)carbonyl]oxy}methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate;
[(2-methyl-3-morpholin-4-ylpropanoyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
(1-methylpiperidine-4-carbonyloxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(1,3-dioxan-5-yl-oxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(1,3-dioxolan-4-ylmethoxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
[(3-hydroxy-2,2-dimethylpropanoyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(4-methoxybenzyloxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(benzyloxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
[(pyridine-4-yl)carbonyloxy]methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate;
{[(1-methyl-1H-imidazol-4-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
[(1,3-dioxan-5-ylcarbonyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(1-methyl-1H-imidazol-5-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
({[(1-methyl-1H-imidazol-4-yl)methoxy]carbonyl}oxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
({[(1-methylpiperidin-4-yl)oxy]carbonyl}oxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
[(1-methylpiperidin-4-yl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
({[(1-methylpiperidin-4-yl)methoxy]carbonyl}oxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(1,3-dioxan-5-ylmethoxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
(Pyridin-3-yloxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(dimethylamino)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(1-amino cyclopropyl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate, trifluoroacetate;
{[(1-methyl-1H-imidazol-2-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-{[(1-methyl-1H-imidazol-5-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-(1-aminocyclopropanecarbonyloxy)ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate, trifloroacetate;
1-({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methoxy)oxo-(2E)-but-2-enoic acid;
{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methyl 1-azabicyclo[2.2.1]heptane-4-carboxylate;
{1-[({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methoxy)carbonyl]cyclopropyl}methanaminium trifluoroacetate;
1-{[(1-methyl-1H-imidazol-4-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-{[(pyridin-3-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-{[(pyridin-2-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methoxy)-4-oxobutanoic acid;
1-({[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}ethoxy)oxo-(2E)-but-2-enoic acid;
(1-methylpiperidin-4-yl)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(1-hydroxycyclopropyl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methyl N-pentanoyl-N-{[2′-(1H-tetrazol-5-yl)biphenyl-4-yl]methyl}-L-valinate;
(4S,5S)-ethyl 5-[(S)-2-(3-amino-2,2-dimethyl-3-oxopropylcarbamoyl)-3-methylbutyl]-4-{(S)-2-[4-methoxy-3-(3-methoxypropoxy)benzyl]-3-methylbutyl}-1,3-oxazolidine-3-carboxylate;
(4S,5S)-1-(isobutyryloxy)ethyl (4S,5S)-5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S)-1-(isobutyryloxy)ethyl (4S,5S)-5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
(4S,5S)—(N-CBz-valyloxy)methyl 5-[(S)-2-(3-amino-2,2-dimethyl-3-oxopropylcarbamoyl)-3-methylbutyl]-4-{(S)-2-[4-methoxy-3-(methoxypropoxy)benzyl]-3-methylbutyl}oxazolidine-3-carboxylate;
{[2-methyl-2-(ethoxymethoxy)propanoyl]oxy}methyl (4S,5S)-5-{(2S)-2-[(3-amino-2,2-dimethyl-3-oxopropyl)carbamoyl]-3-methylbutyl}-4-{(2S)-2-[4-methoxy-3-(3-methoxypropoxy)benzyl]-3-methylbutyl}-1,3-oxazolidine-3-carboxylate;
{[(3-methoxy-2,2-dimethyl-3-oxopropoxy)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
[({1-[(tert-butoxycarbonyl)amino]cyclopropyl}carbonyl)oxy]methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-{1-[(tert-butoxycarbonyl)amino]cyclopropanecarbonyloxy}-ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}methyl tert-butyl (2E)-but-2-enedioate;
{[(1-{[(tert-butoxycarbonyl)amino]methyl}cyclopropyl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}ethyl tert-butyl butanedioate;
1-{[((4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidin-3-yl)carbonyl]oxy}ethyl tert-butyl (2E)-but-2-enedioate;
1-{[(1-methyl-1H-imidazol-5-yl)carbonyl]oxy}ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
1-(Pyridin-3-yloxy)ethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(2-Methylpyridin-3-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(3-Methylpyridin-2-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
{[(4-Methyloxazol-5-yl)carbonyl]oxy}methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
({[1-(Hydroxymethyl)cyclopropyl]carbonyl}oxy)methyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
Pyridine-3-ylmethyl (4S,5S)-5-((2S)-2-{[(3-amino-2,2-dimethyl-3-oxopropyl)amino]carbonyl}-3-methylbutyl)-4-{(2S)-2-[4-methoxy-3-(3-methoxypropoxy)benzyl]-3-methylbutyl}-1,3-oxazolidine-3-carboxylate;
(4S,5S)-ethyl 5-[(S)-2-(3-amino-2,2-dimethyl-3-oxopropylcarbamoyl)-3-methylbutyl]-4-{(S)-2-[4-methoxy-3-(3-methoxypropoxy)benzyl]-3-methylbutyl}-2,2-dimethyloxazolidine-3-carboxylate; and
pharmaceutically acceptable salts thereof.

16. A process for preparing a compound according to claim 1, wherein R¹, R², R³ and R⁴ are H, said process comprising the steps of
a) reacting a compound of formula (II) 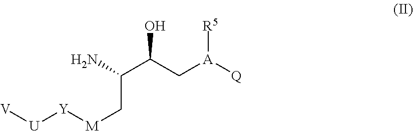
wherein M, Y, U, V, A, R⁵ and Q are as defined in claim 1, with a compound of formula (VIII), 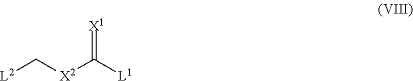
wherein X¹ and X² are as defined in claim 1 and L¹ and L² are leaving groups independently selected from Cl, Br, I, and sulfonates, wherein said reacting is under basic conditions in an inert solvent or mixture of inert solvents to obtain a compound of formula (IX) 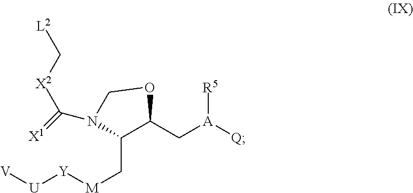
b) subsequently reacting the compound of formula (IX) with a compound of formula (X) or a salt thereof,
R⁶—OH (X)
wherein R⁶ is as defined in claim 1, and wherein said subsequent reacting is under basic conditions in an inert solvent or mixture of inert solvents.

17. A compound of general formula (IX) 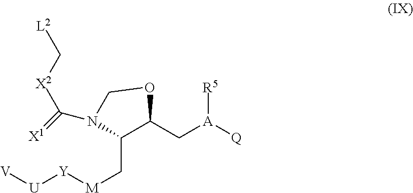
wherein X¹, X², M, Y, U, V, A, R⁵ and Q are as defined in claim 1 and L² is as defined in claim 16.

18. (canceled)

19. A method for treating and/or preventing a renin related disorder in a mammal in need thereof, comprising administering to said mammal a therapeutically effective amount of a compound according to claim 1, or a pharmaceutically acceptable salt thereof.

20. (canceled)

21. A method of treating and/or preventing hypertension, heart failure, glaucoma, cardiac infarction, kidney failure or restenosis in a mammal in need thereof, comprising administering a therapeutically effective amount of a compound according to claim 1, or a pharmaceutically acceptable salt thereof, to said mammal.

22. A pharmaceutical composition comprising a compound according to claim 1, or a pharmaceutically acceptable salt or solvate thereof, in admixture with a pharmaceutically acceptable adjuvant, diluent and/or carrier.

23. A pharmaceutical composition comprising a compound according to claim 1, or a pharmaceutically acceptable salt thereof, and one or more additional agents having cardiovascular action.
